(19) United States
(12) Patent Application Publication
(10) Pub. No.: US 2026/0317761 A1
(43) Pub. Date: Oct. 15, 2026

(54) METHODS OF ASSESSING SPLICE-SWITCHING ASO FEATURES

(71) Applicants: Genentech, Inc., South San Francisco, CA (US); Hoffmann-La Roche Inc., Little Falls, NJ (US)

(72) Inventors: Casper Cassander HOOGENRAAD, San Mateo, CA (US); Amy Elizabeth BYRNES, San Mateo, CA (US); Filip ROUDNICKY, Zurich (CH); Lars JOENSON, Viby Sjaelland (DK)

(73) Assignees: Genentech, Inc., South San Francisco, CA (US); Hoffmann-La Roche Inc., Little Falls, NJ (US)

(21) Appl. No.: 19/418,663

(22) Filed: Dec. 12, 2025

Related U.S. Application Data
(63) Continuation of application No. PCT/US2024/033880, filed on Jun. 13, 2024.
(60) Provisional application No. 63/521,556, filed on Jun. 16, 2023.

(51) Int. Cl. C12N 15/113; C12N 15/85; G01N 33/50

(52) U.S. Cl. CPC C12N 15/113; C12N 15/85; G01N 33/5023; C12N 2310/11; C12N 2320/33

(57) ABSTRACT

Provided herein are methods for assessing splice-switching ASOs using a reporter to detect splicing of a cryptic sequence located in the reporter pre-mRNA.

### CROSS-REFERENCE TO RELATED APPLICATIONS

This application is a continuation of International Application No. PCT/US2024/033880, filed Jun. 13, 2024, which claims benefit of priority to U.S. Patent Application No. 63/521,556, filed Jun. 16, 2023, each of which is incorporated herein by reference in its entirety.

### FIELD

The present methods relate to assessing ASO features by detecting a reporter protein that contains a cryptic sequence within its pre-mRNA.

### REFERENCE TO AN ELECTRONIC SEQUENCE LISTING

This application contains a Sequence Listing which has been submitted electronically in XML format and is hereby incorporated by reference in its entirety. Said XML copy, created on Dec. 10, 2025, is named P38308-US-1_SL.xml and is 30,361 bytes in size.

### BACKGROUND OF THE INVENTION

The use of antisense oligonucleotides (ASOs) as therapeutic agents and research tools has increased dramatically in recent decades. This resurgence in ASO development can be partially attributed to chemistry advancements that enhance ASO stability, circulation time, and protein binding, which together stimulate ASO internalization into cells without the need for transfection reagents and increase drug efficacy by limiting clearance (Khvorova A, Watts J K., Nat Biotechnol., 2017. 35, 238-248). Furthermore, unlike short interfering RNA (siRNA), backbone and sugar-ring modifications can be exploited to promote ASO design-dependent changes in gene expression (Crooke S T. et al., Cell Metab. 2018. 27, 714-739). For example, ASO-mRNA hybridization can either decrease protein expression by recruiting RNase H1 or promote alternative splicing. ASOs can also be designed to alter protein translation using steric-blocking mechanisms or mimic/block micro-RNA function (Schoch K M. et al., Neuron, 2017. 94, 1056-1070). Taken as a whole, these tunable properties have reinvigorated the assessment of pathological targets deemed “undruggable” by conventional small molecules and biologics. Accordingly, ASOs are currently being actively investigated for cardiovascular, hematopathology, neurodegenerative, oncological, and infectious disease indications (Dhuri K. et al., J Clin Med. 2020. 9, 2004).

While ASO research and development has increased dramatically, the underlying mechanisms regulating ASO uptake, dynamics and subsequent activity are poorly understood. Previous studies have shown that ASO-protein binding allows for ASO internalization and trafficking through the endolysosomal system within minutes of exposure (Crooke S T. et al., Nat Biotechnol. 2017. 35, 230-237). However, only a small percentage of internalized ASO molecules escape endolysosomes to become active in the cytoplasm or nucleus (Juliano R L. et al., Nucleic Acids Res. 2016. 44, 6518-48). Furthermore, ASO backbone-mediated toxicities can limit ASO use in vivo due to immune cell activation (Galbraith W M. et al., Antisense Res Dev. 1994. 4, 201-6). Significant advancements in ASO delivery have been hindered partially by a lack of suitable tools for the simultaneous assessment of ASO internalization and activity in vivo. Furthermore, quantifying ASO activity requires both time- and resource-intensive techniques that measure mRNA and protein levels. Thus, developing a high-throughput, quantitative assay to measure ASO delivery as well as activity in a living organism would advance ASO research and facilitate therapeutic development for many diseases.

### SUMMARY

Provided herein is a method of assessing delivery and/or splice-switching activity of an antisense oligonucleotide (ASO) comprising (i) introducing the ASO to a transgenic mouse, wherein the transgenic mouse comprises a plurality of cells which comprise a genetic sequence that is transcribed as a pre-mRNA, said pre-mRNA comprising a first segment encoding a first portion of a reporter protein, a cryptic sequence, and a second segment encoding a second portion of the reporter protein, wherein the ASO alters splicing of the pre-mRNA to exclude the cryptic sequence from a mature mRNA transcript, thereby causing expression of a functional form of the reporter protein, wherein the cryptic sequence comprises a sequence comprising at least 95% sequence identity to the nucleotide sequence set forth in SEQ ID NO.: 1 comprising nucleotide substitutions of T657G and A658T which result in decreased exclusion of the cryptic sequence in the absence of the ASO when compared to exclusion of the cryptic gene without the nucleotide substitutions of T657G and A658T; and (ii) detecting the expression of the reporter protein to assess the delivery and/or splice-switching activity of the ASO in the plurality of cells.

In some embodiments, the amount of ASO that is introduced to the transgenic mouse is about 50 μg or more, optionally wherein the amount of ASO introduced to the transgenic mouse is about 100 μg or more.

In some embodiments, the concentration of ASO that is introduced to the transgenic mouse is about 0.5 μM or more, optionally wherein the concentration of ASO introduced to the transgenic mouse is about 1 μM or more, optionally wherein the concentration of ASO introduced to the transgenic mouse is about 2 μM or more, optionally wherein the concentration of ASO introduced to the transgenic mouse is about 3 μM or more, optionally wherein the concentration of ASO introduced to the transgenic mouse is about 4 μM or more, optionally wherein the concentration of ASO introduced to the transgenic mouse is about 5 μM or more, optionally wherein the concentration of ASO introduced to the transgenic mouse is about 10 μM or more, optionally wherein the concentration of ASO introduced to the transgenic mouse is about 15 μM or more, and optionally wherein the concentration of ASO introduced to the transgenic mouse is about 20 μM or more.

In some embodiments, the sequence that is transcribed as a pre-mRNA is expressed ubiquitously in the transgenic mouse.

In some embodiments, the sequence that is transcribed as a pre-mRNA is expressed in specific tissues or cells.

In some embodiments, the sequence that is transcribed as a pre-mRNA is expressed in neuronal cells, neurons, astrocytes, microglia, cortex, hippocampus, corpus collosum, interneuron, motor neuron, sensory neuron, microglia, astrocytes, brain, oligodendrocyte and/or neuronal tissues.

In some embodiments, the ASO is introduced to the transgenic mouse by injection, inhalation, intracerebroventricular injection, intravitreal injection, intravenous injection, intraperitoneal injection, or intrathecal injection and optionally wherein the ASO is administered directly to the central nervous system or the cerebrospinal fluid of the mammal.

In some embodiments, the cryptic sequence comprises the nucleotide sequence set forth in SEQ ID NO.: 1.

Provided herein, in some embodiments, is a method of assessing delivery and/or splice-switching activity of an antisense oligonucleotide (ASO) comprising: (i) introducing the ASO to a plurality of cultured cells, wherein the plurality of cultured cells are isolated from a transgenic animal comprising a genetic sequence that is transcribed as a a pre-mRNA, said pre-mRNA comprising a first segment encoding a first portion of a reporter protein, a cryptic sequence, and a second segment encoding a second portion of the reporter protein, wherein the ASO alters splicing of the pre-mRNA to exclude the cryptic sequence from a mature mRNA transcript, thereby causing expression of a functional reporter protein, wherein the cryptic sequence comprises a sequence comprising at least 95% sequence identity to the nucleotide sequence of SEQ ID NO.: 1 comprising nucleotide substitutions of T657G and A658T which result in decreased exclusion of the cryptic sequence in the absence of the ASO when compared to exclusion of the cryptic gene without the nucleotide substitutions of T657G and A658T, and (ii) detecting the expression of the reporter protein to assess the delivery and/or activity of the ASO in the plurality of cultured cells.

In some embodiments, the transgenic animal is a mammal. In some embodiments, the mammal is a mouse or a rat.

In some embodiments, the cryptic sequence comprises the nucleotide sequence set forth in SEQ ID NO.: 1.

Provided herein, in some embodiments, is a method of assessing a feature of an antisense oligonucleotide (ASO) in a plurality of cells comprising: (i) providing the ASO to the plurality of cells, wherein the plurality of cells comprises a genetic sequence that is transcribed as a pre-mRNA, said pre-mRNA comprising a first segment encoding a first portion of a reporter protein, a cryptic sequence, and a second segment encoding a second portion of the reporter protein, wherein the ASO alters splicing of the pre-mRNA to exclude the cryptic sequence from a mature mRNA transcript, thereby causing expression of a functional reporter protein, wherein the cryptic sequence comprises one or more nucleotide insertions, deletions, or substitutions that result in decreased exclusion of the cryptic sequence in the absence of the ASO when compared to exclusion of the cryptic gene without said one or more nucleotide insertions, deletions, or substitutions, and (ii) detecting the expression of the reporter protein to assess a feature of the ASO in the plurality of cells.

In some embodiments, the cryptic sequence comprises a sequence of SEQ ID NO.: 1 comprising nucleotide substitutions of T657G and A658T which result in the decrease of the exclusion of the cryptic sequence in the absence of the ASO.

In some embodiments, the plurality of cells are mammalian cells. In some embodiments, the mammalian cells are human, rat, or mouse cells.

In some embodiments, the plurality of cells are from the same tissue.

In some embodiments, the reporter protein is EGFP or luciferase.

In some embodiments, the nucleotide substitutions of T657G and A658T decrease the exclusion of the cryptic sequence in the absence of splice-switching ASO by at least 10%, 20%, or 30%.

In some embodiments, the ASO comprises a nucleotide sequence that is complementary to a part of the cryptic sequence, optionally wherein the ASO is complementary to a splice donor site or a splice acceptor site that regulates splicing of the cryptic sequence.

In some embodiments, the reporter protein is detected in a living cell or animal.

In some embodiments, wherein the plurality of cells or the plurality of cultured cells comprise neuronal tissue, neurons, neurons, astrocytes, microglia, cortex, hippocampus, corpus collosum, interneurons, motor neuron, sensory neuron, microglia, astrocytes, and/or oligodendrocyte.

In some embodiments, the ASO comprises one or more chemical modifications.

In some embodiments, the one or more chemical modifications comprise a modification to a phosphate backbone and/or a modification to a sugar, and optionally wherein, the chemical modification comprises 2-MOE, 2′-OMe, LNA, GalNAc, 5′ methylcytosine, a phosphorothioate bond, a phosphorodithioate bond, an alkylphosphonate bond, a phosphoroamidate bond, a boranophosphate bond, and/or a morpholino ring.

In some embodiments, the ASO is 12 to 30 nucleotides in length, optionally wherein the ASO is 14 to 18 nucleotides in length, and optionally wherein the ASO is 16 nucleotides in length.

In some embodiments, the amount of ASO that is introduced to the plurality of cultured cells is about 1 μg or more, optionally wherein the amount of ASO introduced to the plurality of cultured cells is about 10 μg or more, optionally wherein the amount of ASO introduced to the plurality of cultured cells is about 50 μg or more, optionally wherein the amount of ASO introduced to the plurality of cultured cells is about 100 μg or more, optionally wherein the amount of ASO introduced to the plurality of cultured cells is about 150 μg or more, and optionally wherein the amount of ASO introduced to the plurality of cultured cells is about 200 μg or more.

In some embodiments, the concentration of ASO that is introduced to the plurality of cells or the plurality of cultured cells is about 0.5 μM or more, optionally wherein the concentration of ASO introduced to the plurality of cultured cells is about 1 μM or more, optionally wherein the concentration of ASO introduced to the plurality of cultured cells is about 2 μM or more, optionally wherein the concentration of ASO introduced to the plurality of cultured cells is about 3 μM or more, optionally wherein the concentration of ASO introduced to the plurality of cultured cells is about 4 μM or more, and optionally wherein the concentration of ASO introduced to the plurality of cultured cells is about 5 μM or more. In some embodiments, the concentration of ASO introduced into the plurality of cultured cells is about 0.5 μM, 0.625 μM, 1.0 μM, 1.25 μM, 2.5 μM, 5 μM, 10 μM, or 20 μM.

In some embodiments, the detected expression of the reporter is indicative of presence, amount, and/or splice-switching activity of the ASO in the cell.

In some embodiments, the feature is a formulation, a clearance rate, a delivery route, pharmacokinetics, pharmacodynamics, uptake, tissue localization, concentration, cellular internalization, impact on gene expression, a modification, impact of genetics on ASO activity, cellular trafficking, or biodistribution.

In some embodiments, providing the ASO to the cell comprises providing the ASO to a cell in a cell culture, optionally wherein the cell is a mammalian cell, and optionally wherein the cell is a human cell, a rat cell, or a mouse cell.

In some embodiments, the ASO is provided to a mammal comprising the plurality of cells, optionally wherein the ASO is administered to the animal by injection or by intracerebroventricular injection, intrathecal injection, intravitreal injection, intravenous injection, or intraperitoneal injection, and optionally wherein the ASO is administered directly to the central nervous system or the cerebrospinal fluid of the mammal.

In some embodiments, the plurality of cells or the comprises a DNA cassette comprising the genetic sequence. In some embodiments, the DNA cassette is integrated into the genome of the cell at a safe harbor locus. In some embodiments, the DNA cassette is integrated at the ROSA26 locus. In some embodiments, the DNA cassette further comprises a polynucleotide encoding a second reporter protein. In some embodiments, the second reporter protein is a fluorescent or luminescent protein. In some embodiments, the second reporter protein is not EGFP or luciferase. In some embodiments, the second reporter protein is mKate.

In some embodiments, the DNA cassette comprises a promoter operably linked to the nucleic acid that is transcribed as the pre-mRNA. In some embodiments, the promoter is operably linked to the first or second reporter protein. In some embodiments, the promoter is constitutive. In some embodiments, the promoter is inducible. In some embodiments, the promoter is tissue-specific. In some embodiments, the promoter is specific to neuronal cells.

In some embodiments, the promoter is a ubiquitous promoter. In some embodiments, the promoter is a CMV early enhancer/chickenβ-actin (CAG) promoter.

In some embodiments, the splice-switching ASO is complementary to a 5′ splice donor site of the cryptic sequence. In some embodiments, the cryptic sequence is a cryptic (3-globin (HBB). In some embodiments, the cryptic sequence comprises the sequence set forth in SEQ ID NO: 1.

In some embodiments, the splice-switching ASO is complementary to SEQ ID NOs: 17-20.

In some embodiments, the method comprises testing a plurality of splice-switching ASOs. In some embodiments, each of the plurality of splice-switching ASOs comprises the same nucleotide sequence. In some embodiments, each of the plurality of splice-switching ASOs comprises a nucleotide sequence that is complementary to the cryptic exon. In some embodiments, the plurality of splice-switching ASOs differ with respect to formulation, chemical modification, delivery method, concentration, or amount.

In some embodiments, the plurality of splice-switching ASOs are assessed in a high-throughput system. In some embodiments, the high-throughput system comprises one or more automated processes.

In some embodiments, the high throughput system comprises cell culture and/or detection of the luciferase and/or EGFP in a 96 well or 364 well format.

Provided herein, in some embodiments, is a kit comprising a transgenic mouse comprising a plurality of cells which comprise a genetic sequence that is transcribed as a pre-mRNA which comprises a first segment encoding a first portion of a reporter protein, a cryptic sequence, and a second segment encoding a second portion of the reporter protein; or a plurality of cells which comprise a genetic sequence that is transcribed as a pre-mRNA which comprises a first segment encoding a first portion of a reporter protein, a cryptic sequence, and a second segment encoding a second portion of the reporter protein; and instructions for assessing a feature of an ASO by detecting the reporter protein according to any of the methods described herein. In some embodiments, the kit further comprises a plurality of ASOs.

### BRIEF DESCRIPTION OF THE DRAWINGS

The drawings illustrate certain features and advantages of this disclosure. These embodiments are not intended to limit the scope of the claims in any manner.

FIG. 1A depicts the structure of the split EGFP cassette (ssEGFP.HBB). A human β-globulin (HBB) cryptic exon interrupts the EGFP coding sequence that is split into two regions, 1 and 2. Splice-switching ASOs sterically block an aberrant splice site, restoring pre-mRNA splicing and producing functional EGFP protein.

FIG. 1B depicts a FACS assessment of ssEGFP.HBB expressing cells without ASO treatment. Cells lacking and containing the 1128-1129 TA to GT mutation are shown on the left and right, respectively. The 1128-1129 TA to GT mutations reduces background EGFP signal, thus limiting construct leakiness.

FIG. 1C depicts the construct used to test ASO-mediated EGFP splicing correction in HEK293 cells.

FIG. 1D depicts results from a FACS analysis in which the construct described in FIG. 1C was transfected into NLS-CRE expressing HEK293 cells and stable cells were obtained using serial FACS sorting for mKate2-positive cells over one month.

FIG. 1E depicts representative images showing mKate2 and EGFP expression in the HEK293 cells described in FIG. 1D 2 days after transfection with ASOs. EGFP splice-switching was observed with active ssASO1, ssASO2 and ssASO3, but not in non-treated or negative control ASO-treated cells (ssNC1). Scale bars, 100 μm.

FIG. 2A depicts the construct used to create a transgenic mouse expressing ssEGFP.HBB. The transgene was inserted at the ROSA26 safe harbor locus and contains mKate2 followed by ssGFP.HBB, which is driven by a CAG promoter.

FIG. 2B depicts the body weight of wildtype (wt), heterozygous (het), and knock-in (ki) transgenic animals.

FIG. 2C depicts representative images of mKate2 fluorescence expression in live wildtype (wt), heterozygous (het), and knock-in (ki) animals.

FIG. 2D depicts quantification of mKate2 fluorescence in the tail, back paw, front paw, nose, and ear.

FIG. 2E depicts ex vivo brain, spleen, kidney, liver, heart, and skin (ear) tissue weights from wt, het, and ki animals.

FIG. 2F depicts representative images of mKate fluorescence in brain, spleen, kidney, liver, heart, and skin tissues obtained from wt, het, and ki animals.

FIG. 2G depicts quantification of mKate2 fluorescence intensity in the tissues shown in FIG. 2F.

FIG. 2H depicts a representative image of EGFP background signal in live transgenic mice.

FIG. 2I-J depicts quantification of EGFP background in live mice (FIG. 2I) and ex vivo tissue samples (FIG. 2J) from wildtype (wt), heterozygous (het), and knock-in (ki) animals. Fluorescence was measured using the same box size across each body part/tissue sample and all scale bars are 0.5 inches. Mean±SEM are shown and one-way ANOVA with a Tukey's multiple comparisons test was used, *p<0.0332, **p<0.0021, ***p<0.0002, ****p<0.0001.

FIG. 3A depicts representative images showing mKate2 is specifically expressed in neurons, microglia, and astrocytes cultured from ki ssEGFP.HBB transgenic mice.

FIGS. 3B-3D depict dose-response curves analyzing the percent of EGFP-positive neurons (FIG. 3B), microglia (FIG. 3C), and astrocytes (FIG. 3D) after 7 days of ASO treatment. Percent EGFP-positive cells was calculated using DAPI-EGFP colocalization. The experiment was completed on three-independent cell cultures. Neuronal cell death was observed at high concentrations of ssASO2 and ssASO3, stars.

FIG. 3E depicts representative images showing EGFP expression in neurons, microglia, and astrocytes after 0, 1, 4, and 7 days of ssASO1 gymnosis.

FIG. 3F depicts quantification of EGFP fluorescence over time. Total fluorescence intensity was normalized to the number of cells per image, as determined using DAPI. Ten images were analyzed per timepoint and the experiment was completed on two-independent cell cultures. Mean±SEM are shown and one-way ANOVA with multiple comparisons Dunnett's test (day 0) was used, ***p<0.0002, ****p<0.0001. All scale bars, 100 μm.

FIG. 4A depicts an exemplary experimental approach wherein transgenic ssEGFP.HBB ki male mice were intracerebroventricularly injected (ICV) injected with saline (n=6), 100 μg ssASO1 (n=6), or 200 μg ssASO1 (n=3), and analyzed over 14 days.

FIG. 4B depicts toxicity post injection of saline, 100 μg ssASO1, and 200 μg ssASO1.

FIG. 4C depicts percent body weight change normalized to day 0 in transgenic ssEGFP.HBB ki male mice that were intracerebroventricularly injected (ICV) injected with saline (n=6), 100 μg ssASO1 (n=6), or 200 μg ssASO1 (n=3), and analyzed over 14 days.

FIG. 4D depicts representative fluorescence IHC images of IBA1 and GFAP staining in the brain of ICV-injected mKate2.ssEGFP.HBB animals 7 and 14 days post injection. Scale bars, 200 μm.

FIG. 4E depicts quantification of IBA1 fluorescence intensity per area (left) and percent IBA1 positive area (right). Three cortical images were acquired and analyzed, and the resulting means were plotted for each treatment (n=3). Mean±SEM are shown, and an unpaired student's t-test (Day 7) or one-way ANOVA with multiple comparisons Tukey's test (Day 14) were used, *p<0.05.

FIG. 4F depicts quantification of GFAP fluorescence intensity per area (left) and percent GFAP positive area (right). Three cortical images were acquired and analyzed, and the resulting means were plotted for each treatment (n=3). Mean±SEM are shown, and an unpaired student's t-test (Day 7) or one-way ANOVA with multiple comparisons Tukey's test (Day 14) were used, *p<0.05.

FIG. 4G depicts representative images of mKate2 (top) and EGFP (bottom) fluorescence expression in live, saline or ssASO1-injected, ssEGFP.HBB ki mice 7 and 14 days post ICV injection.

FIGS. 411-4I depict quantification of mKate2 (FIG. 4E) and EGFP (FIG. 4F) fluorescence in the tail, back paw, front paw, nose, ear, and head. Data for each condition is presented, from left to right, as Day 7 Saline, Day 7 ssASO1, Day 14 Saline, and Day 14 ssASO1.

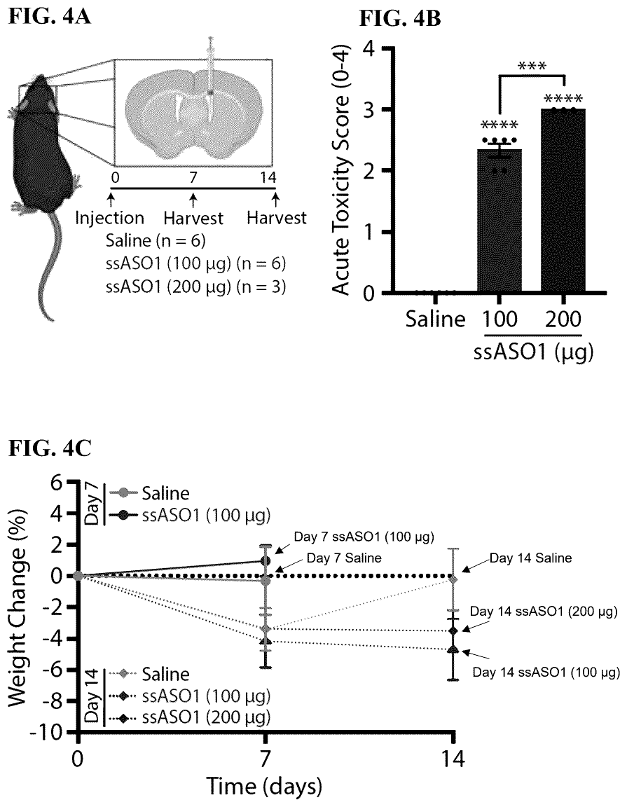

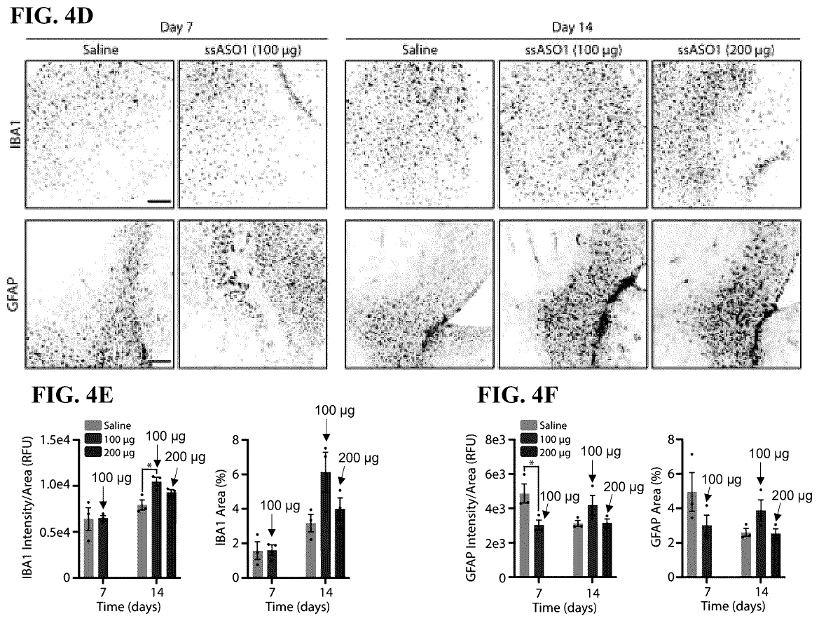

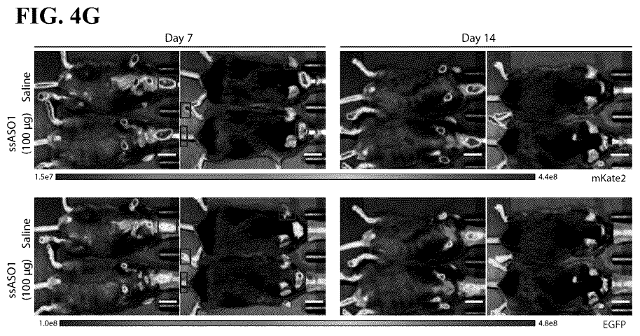

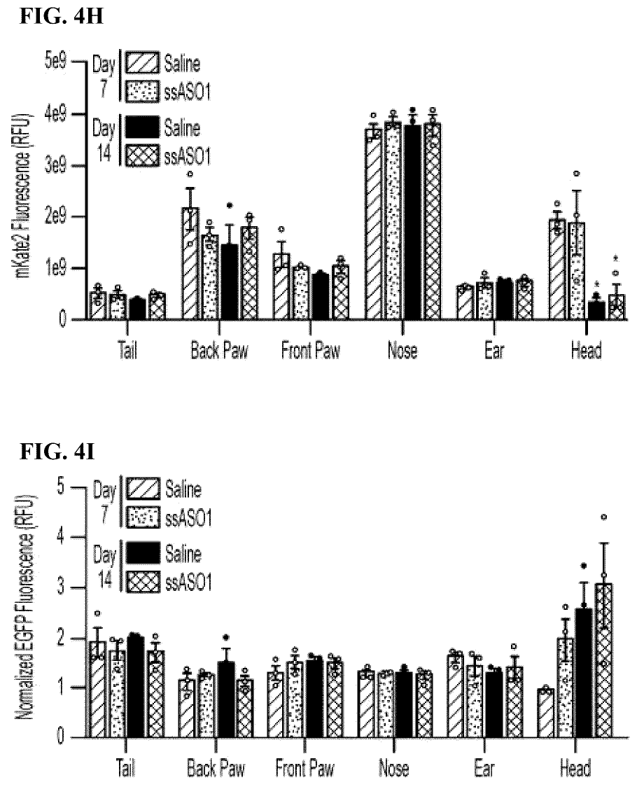

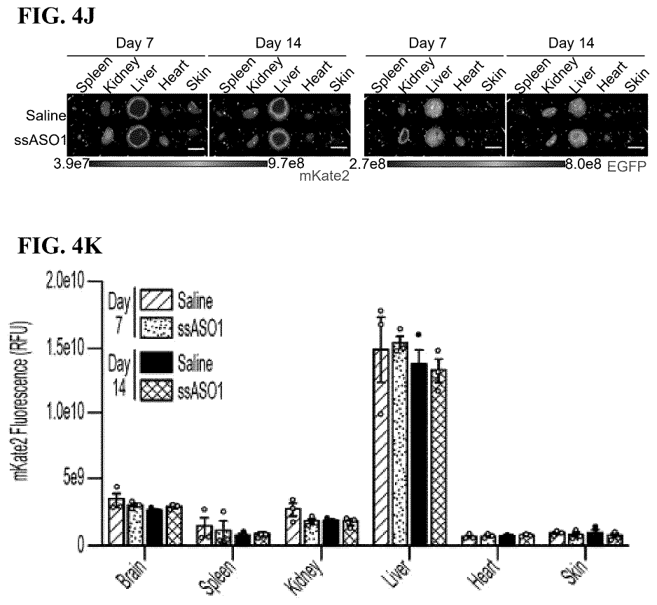

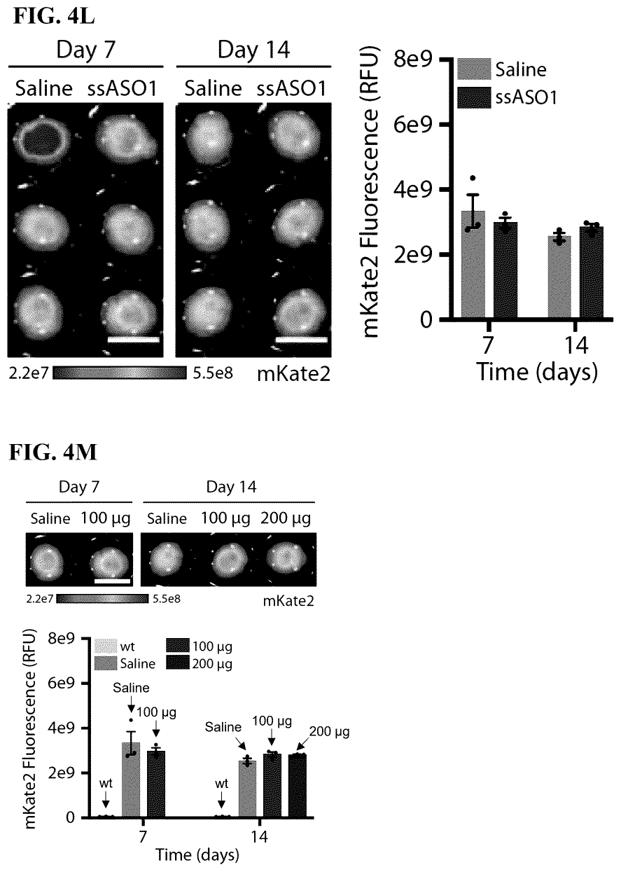

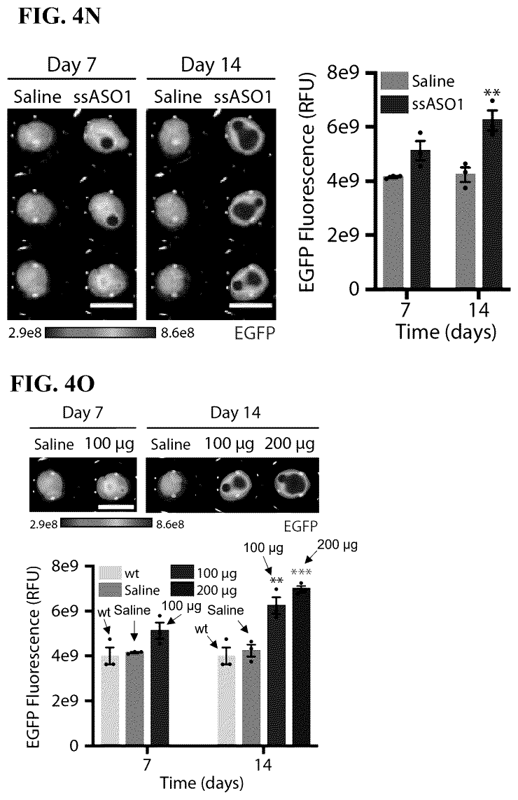

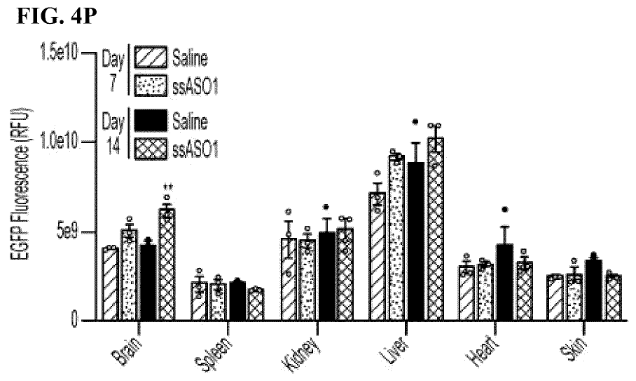

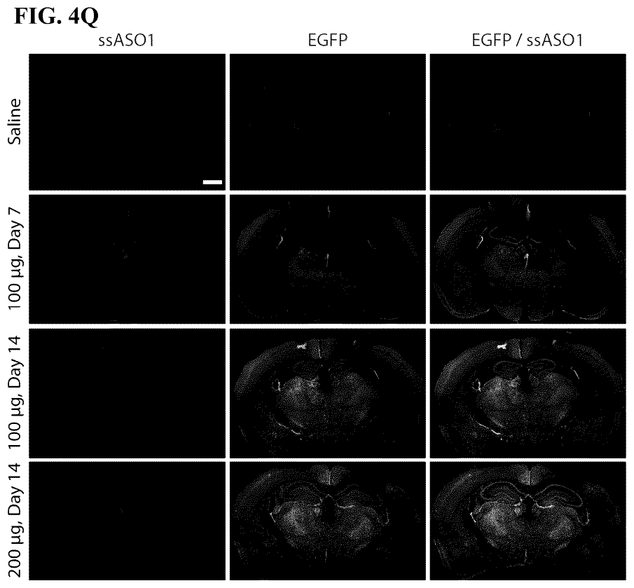

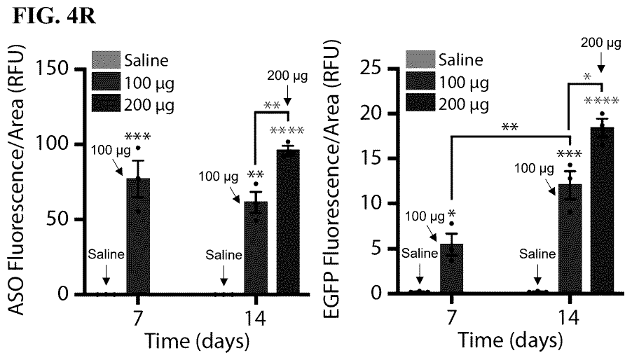

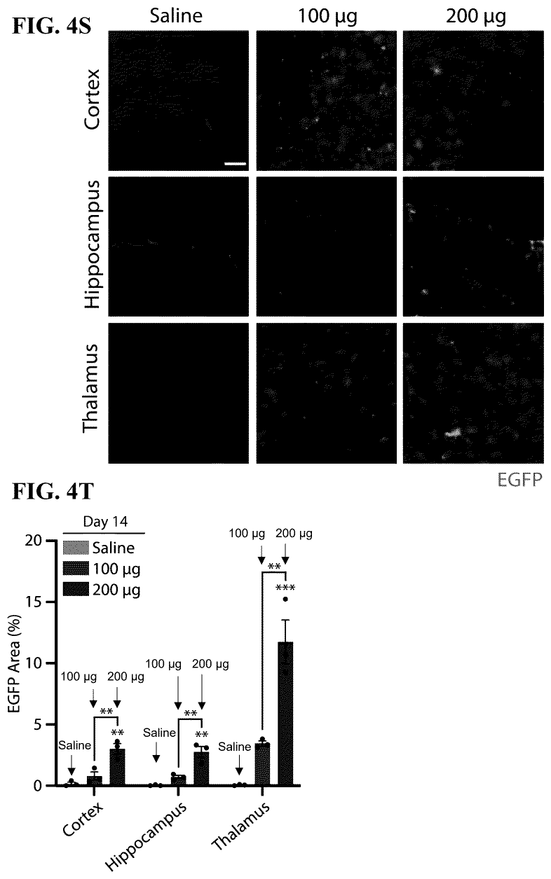

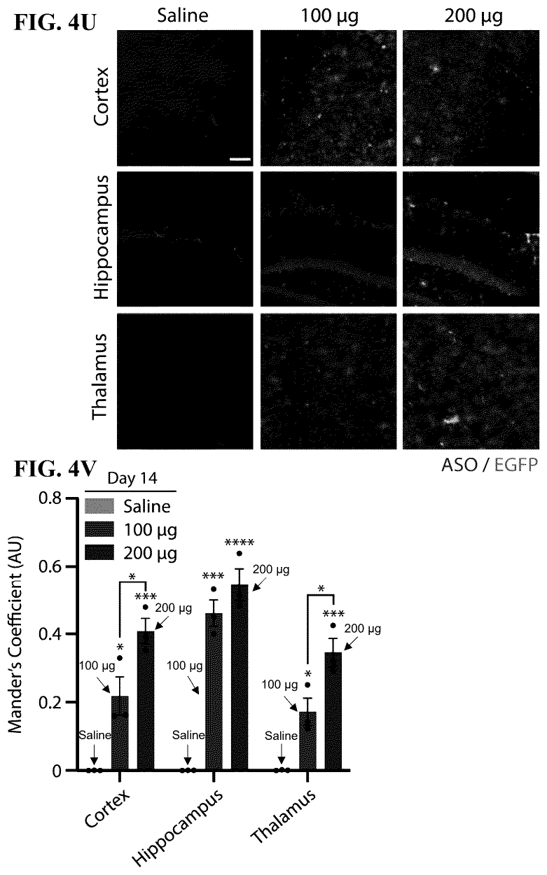

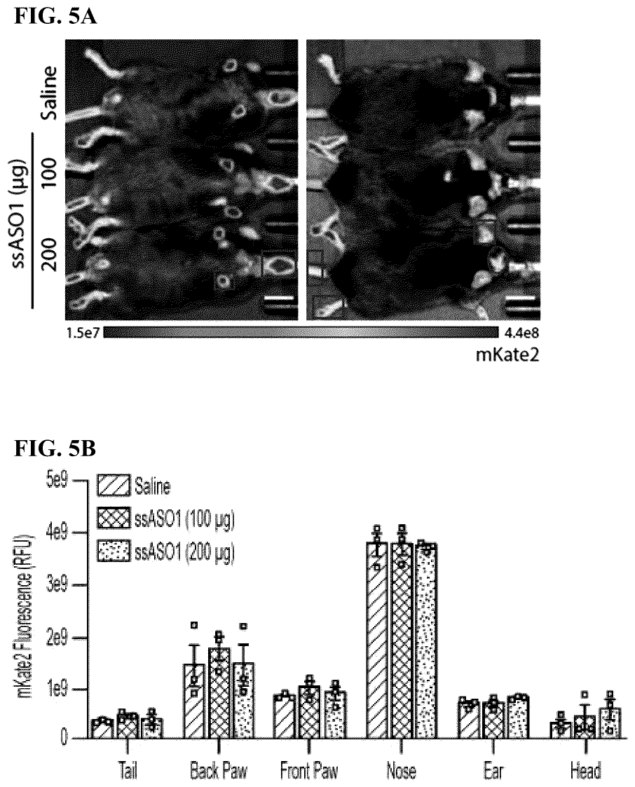

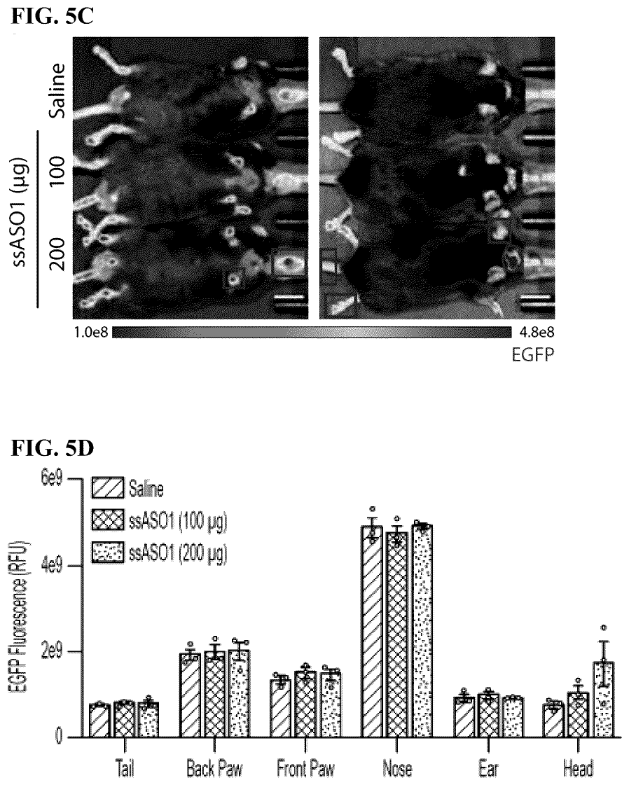

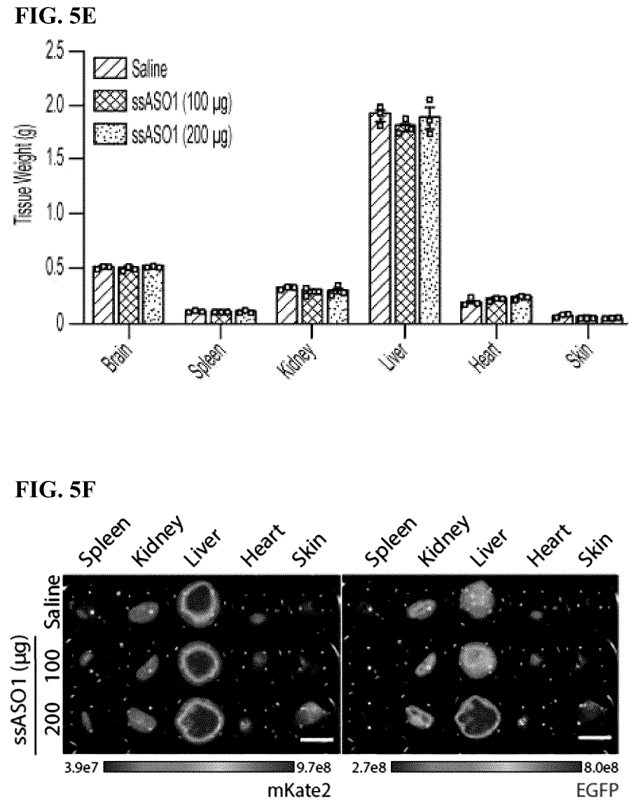

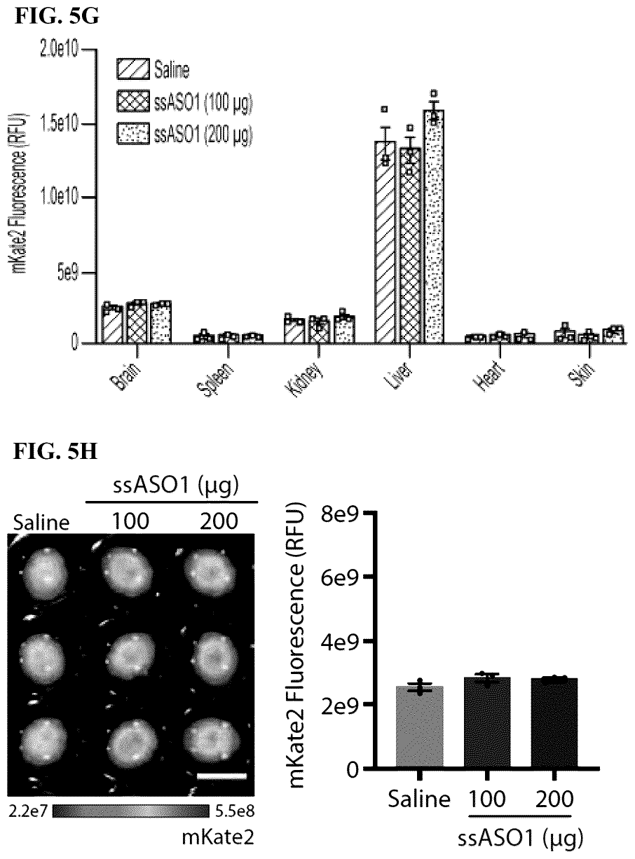

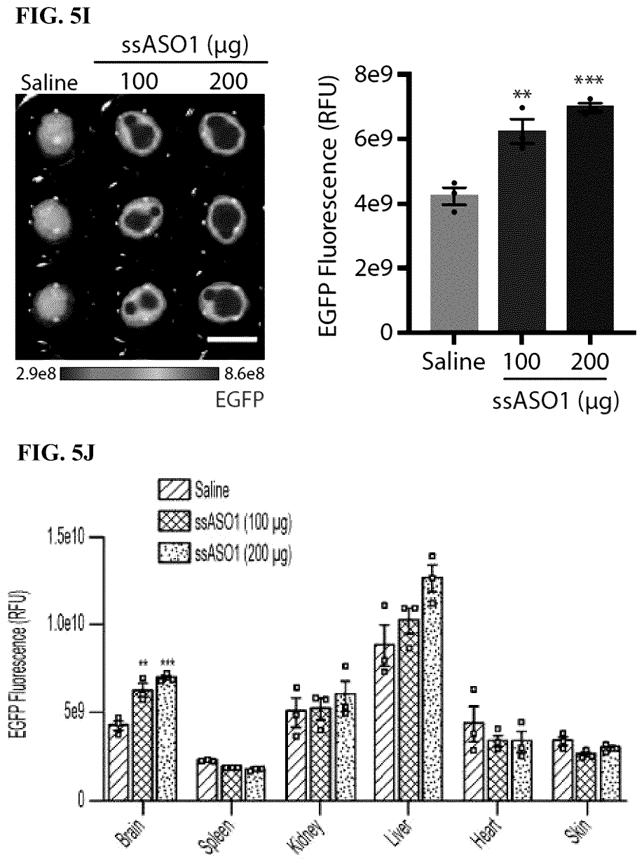

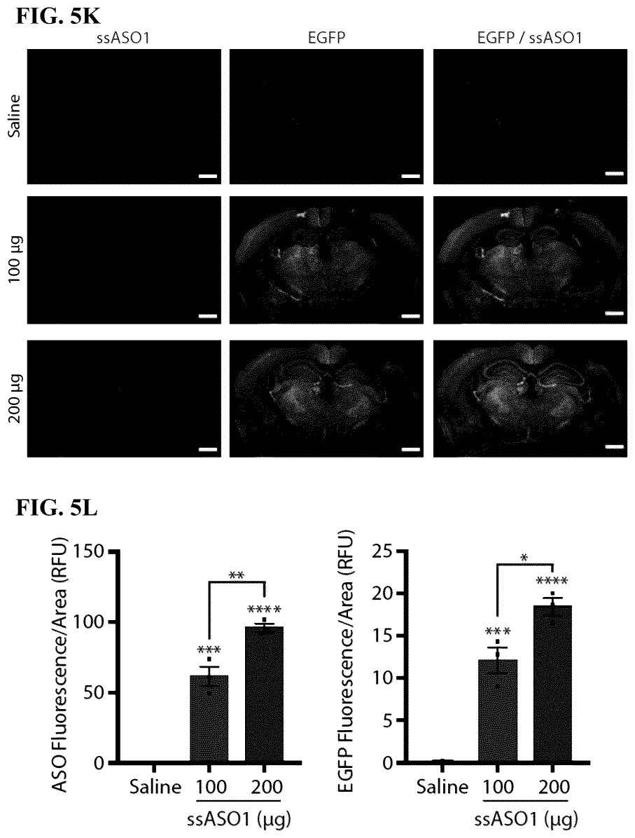

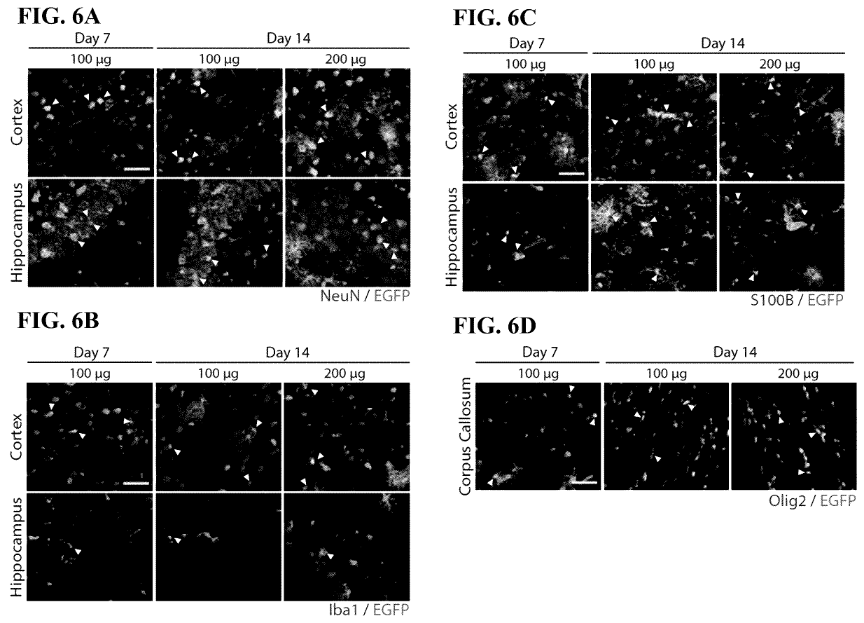

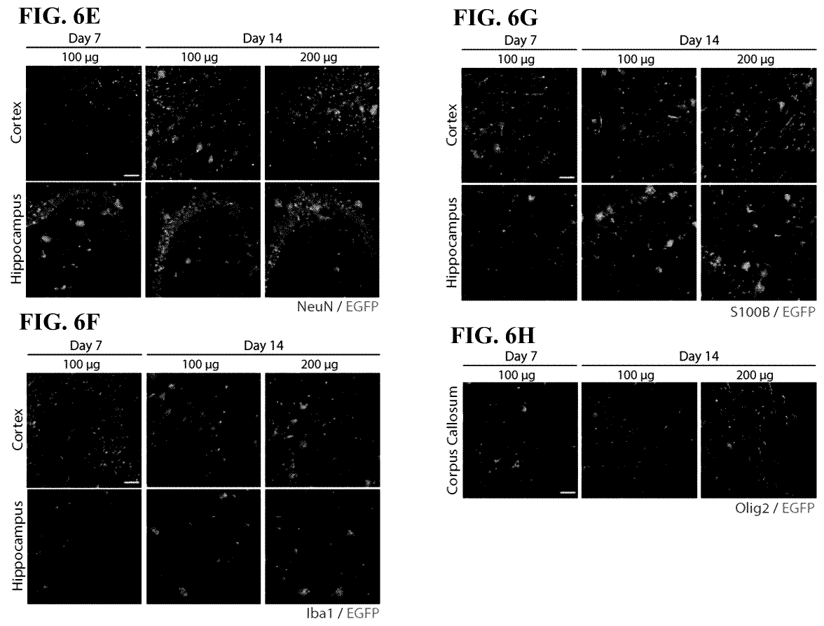

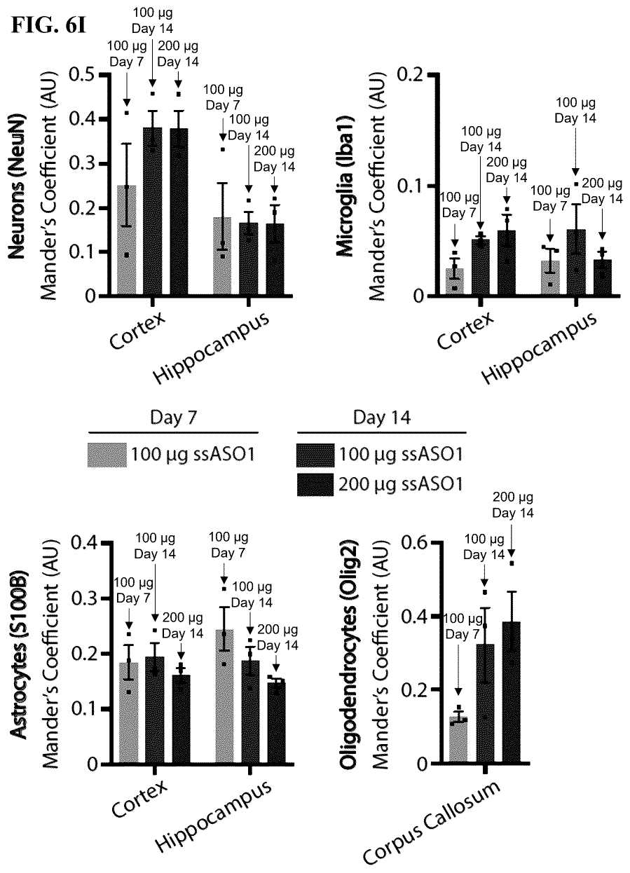

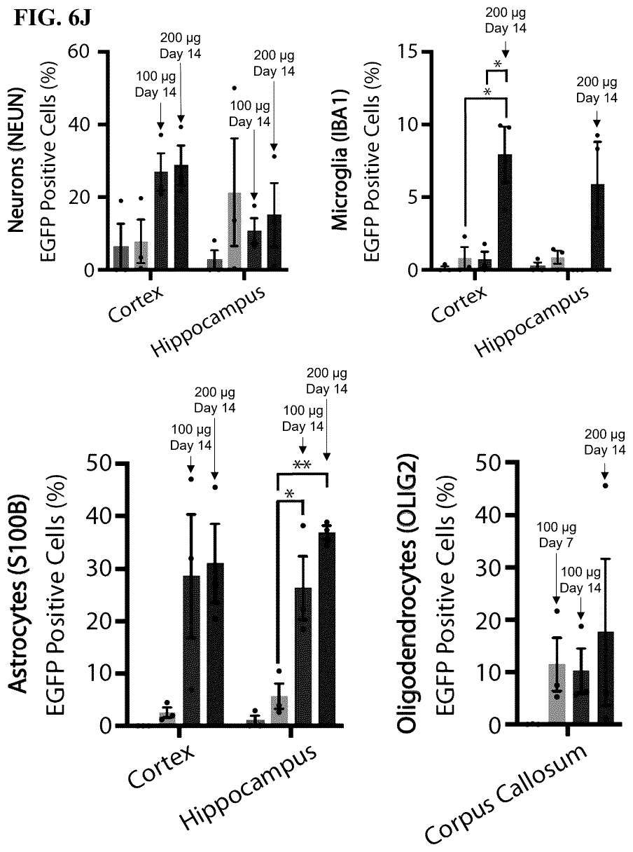

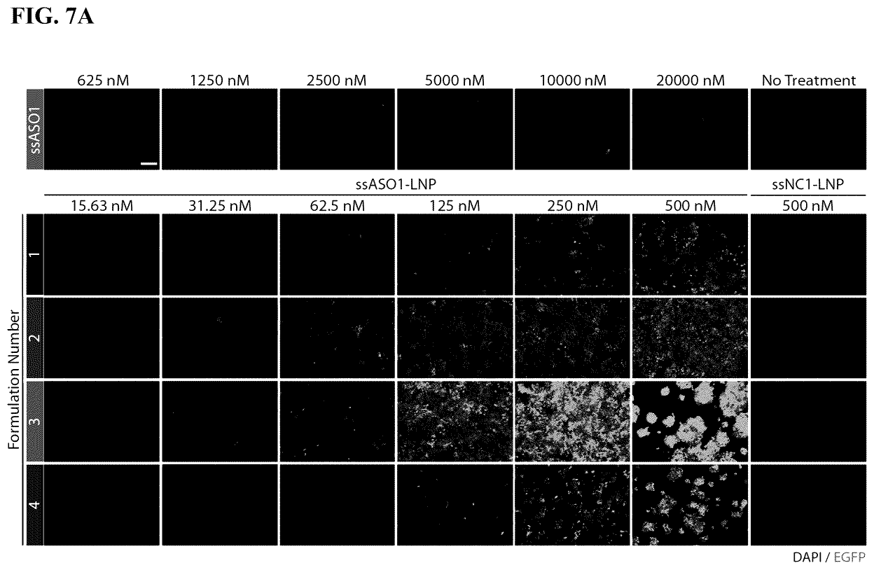

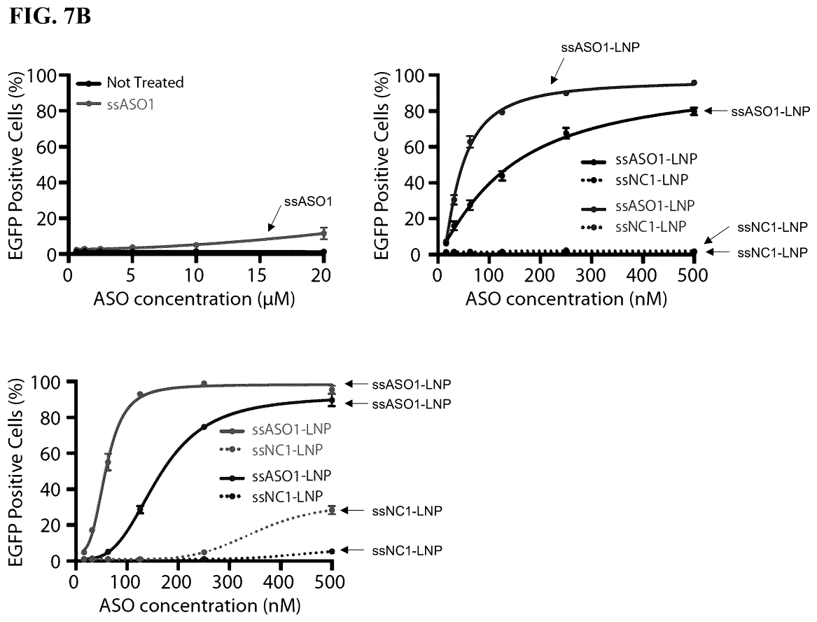

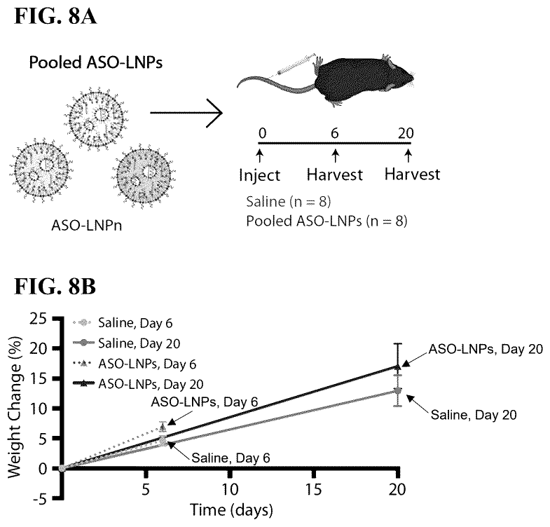

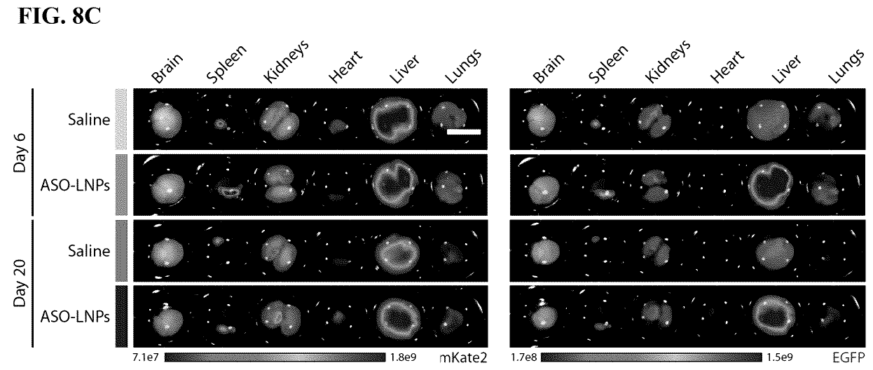

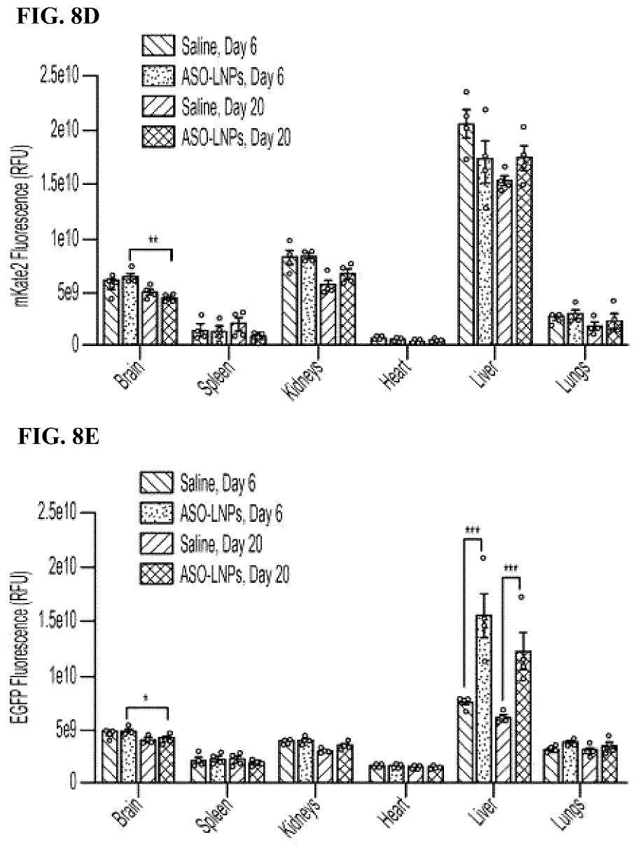

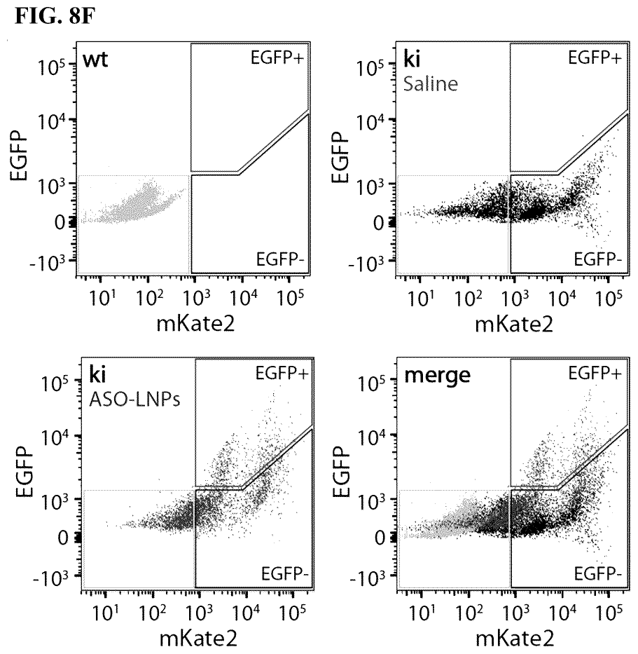

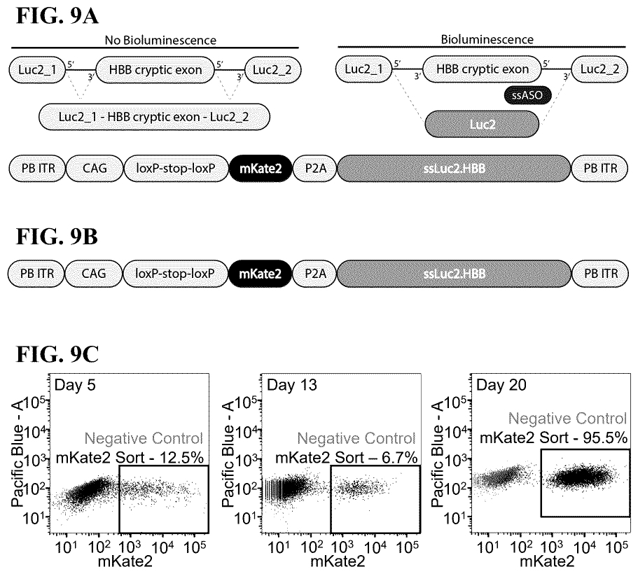

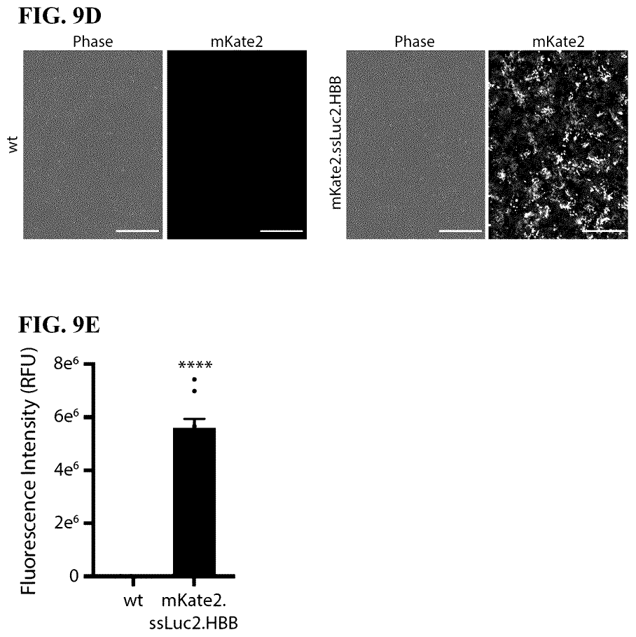

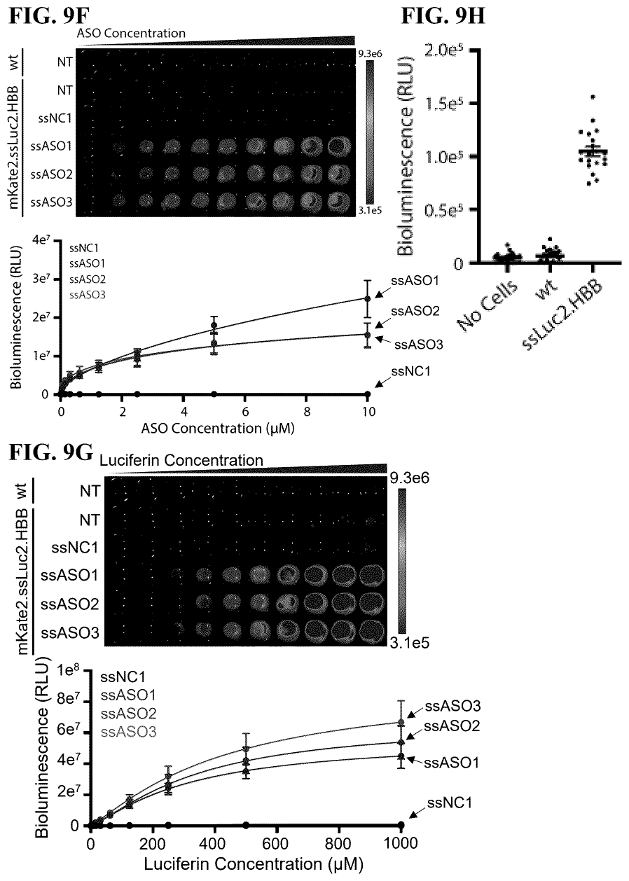

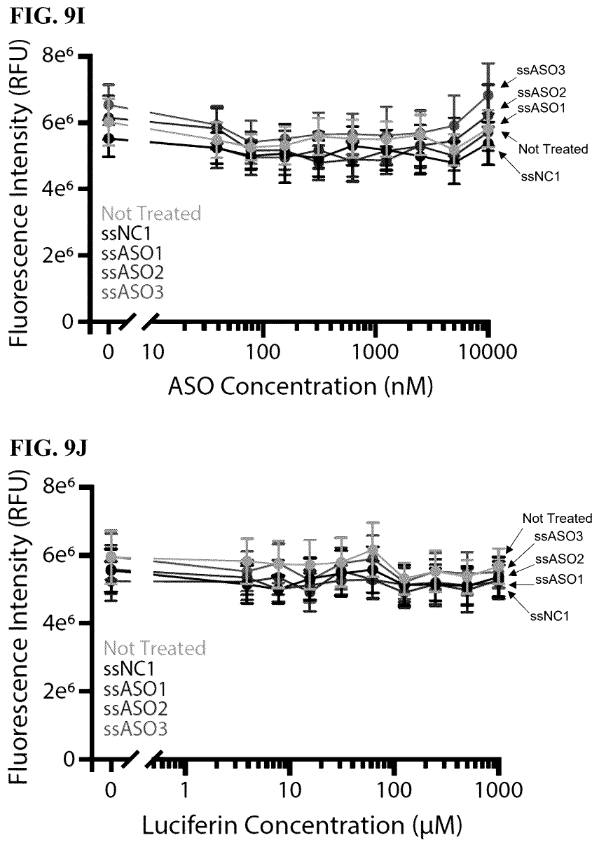

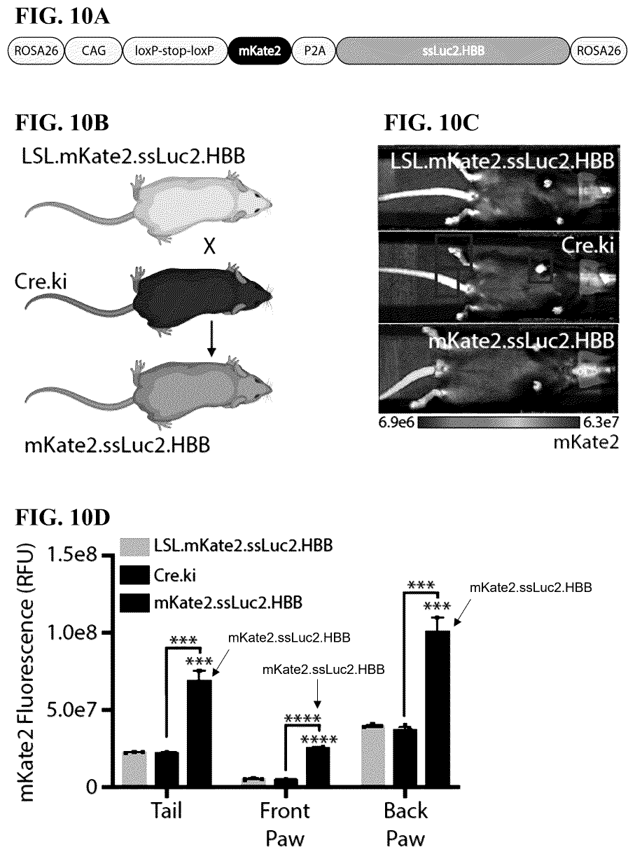

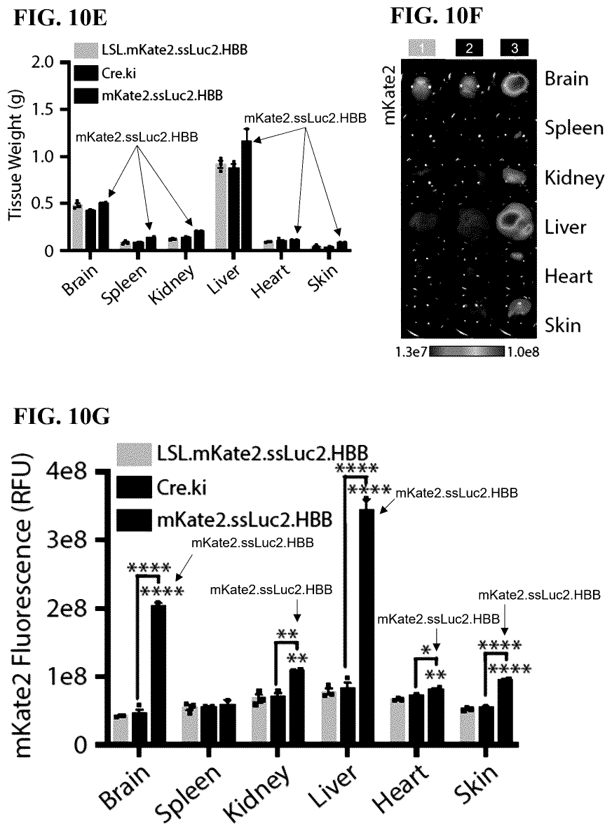

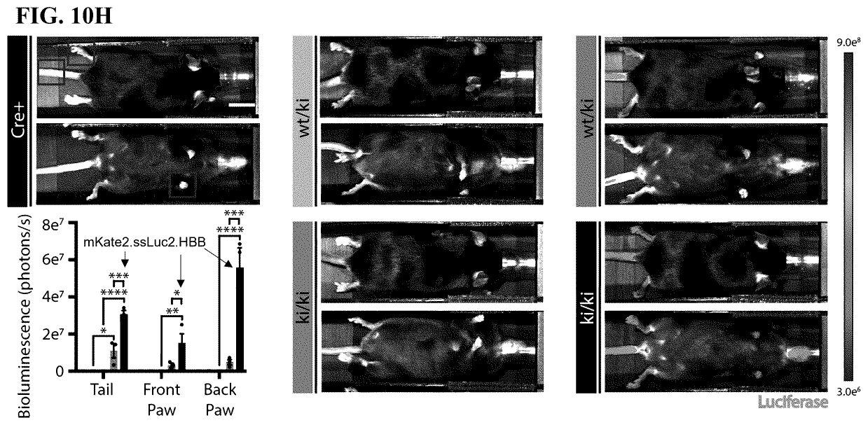

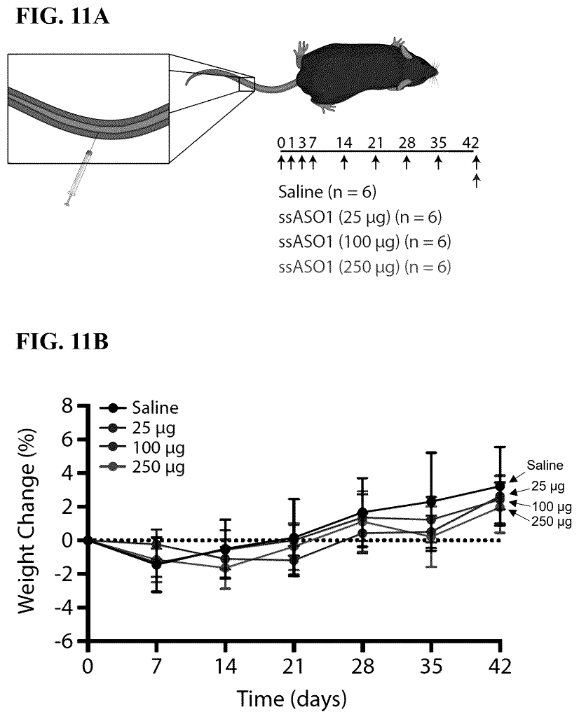

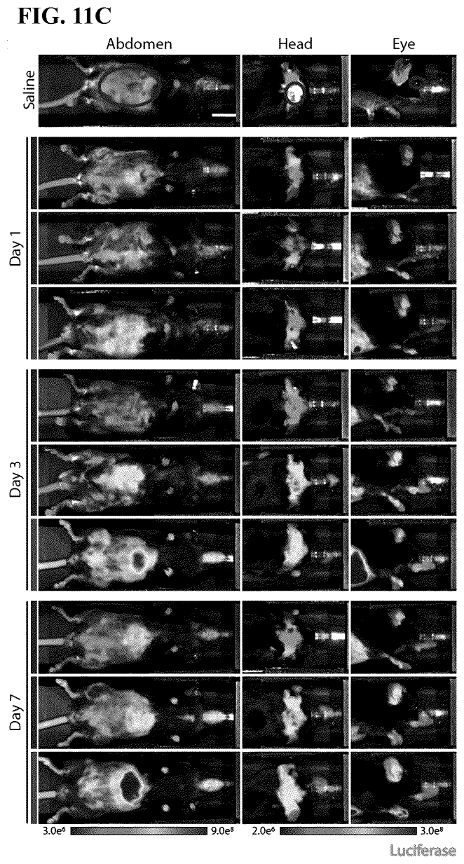

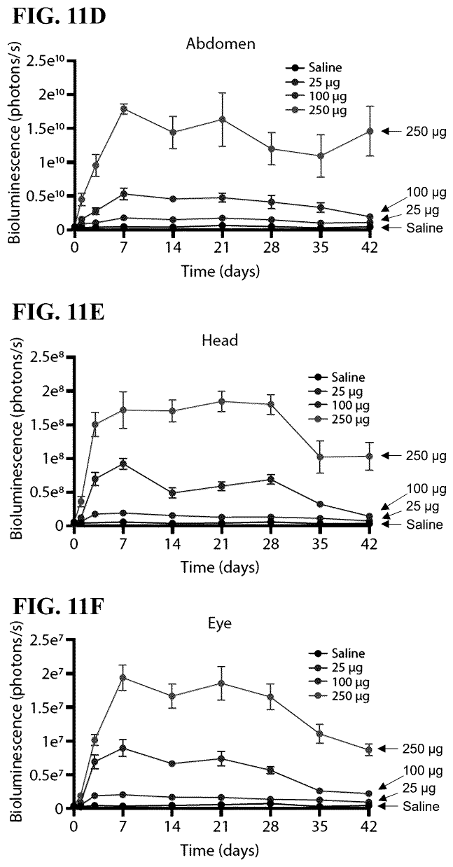

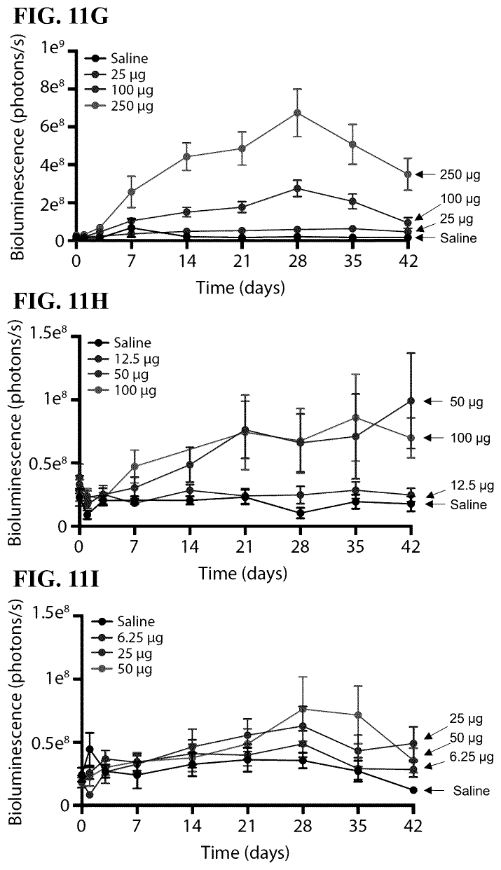

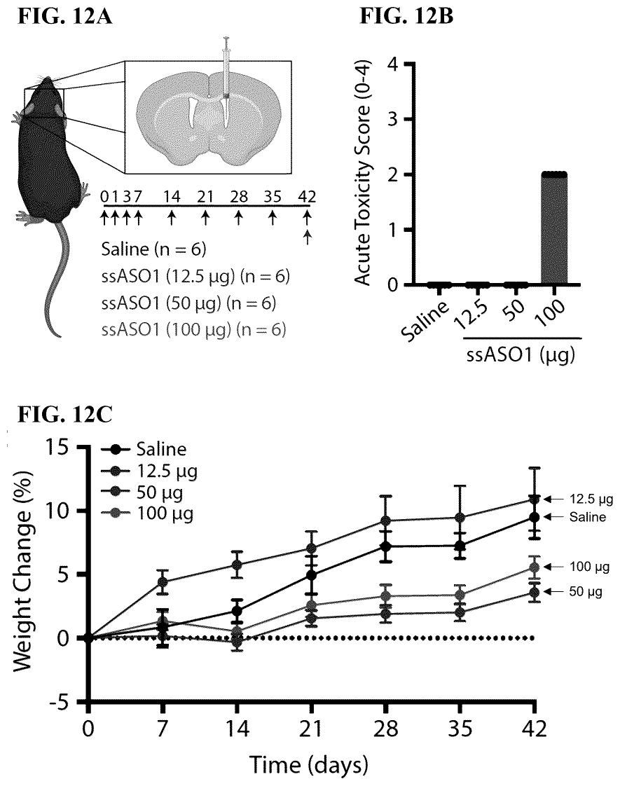

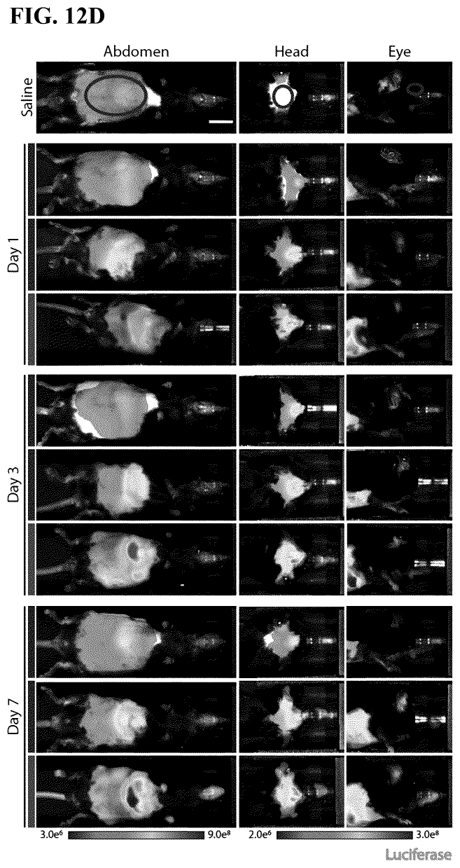

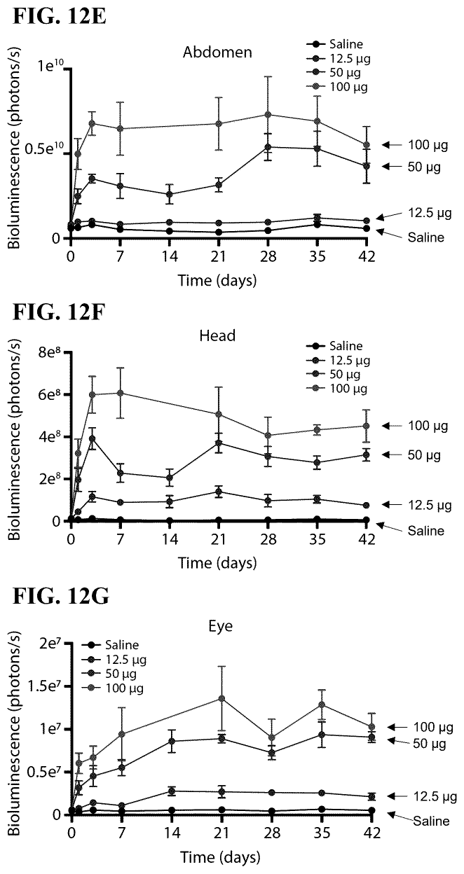

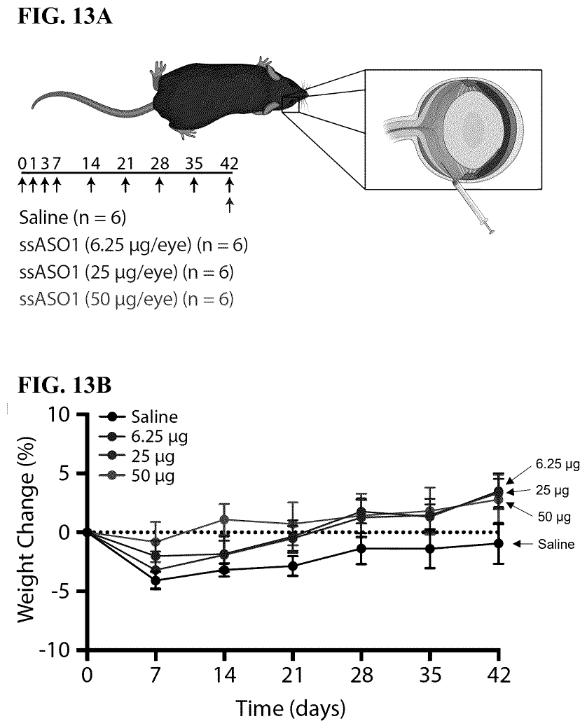

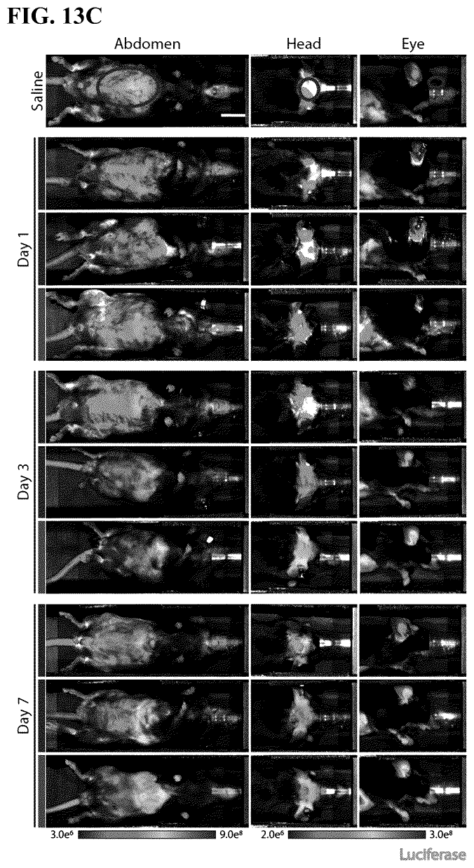

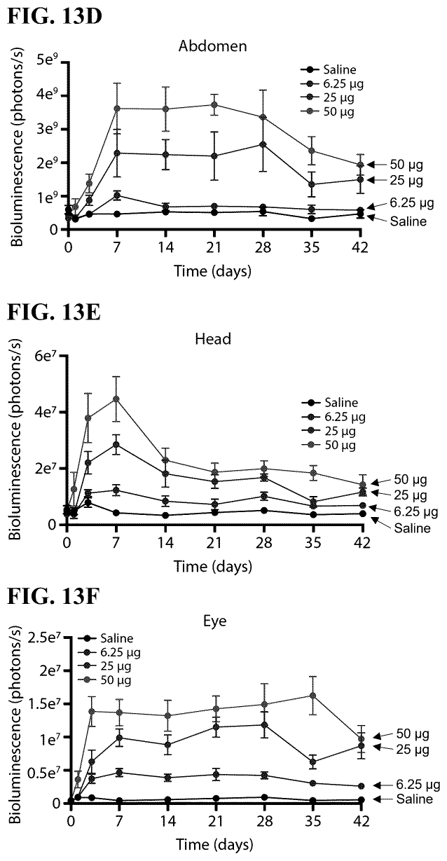

FIG. 4J depicts representative ex vivo mKate2 (left) and EGFP (right) tissue fluorescence imaging following ICV injection.

FIG. 4K depicts quantification of mKate2 fluorescence in the brain, spleen, kidney, liver, heart, and skin (ear). Data for each condition is presented, from left to right, as Day 7 Saline, Day 7 ssASO1, Day 14 Saline, and Day 14 ssASO1.

FIG. 4L depicts representative ex vivo mKate2 brain fluorescence images (Day 7, left, and Day 14, center) and quantification (right). Scale bars, 0.5 inches.

FIG. 4M depicts representative ex vivo mKate2 brain tissue fluorescence imaging (Day 7, top left, and Day 14, top right) and quantification (bottom) post ICV injection. Scale bars, 0.5 inches. Data from WT animals were added for comparison only.

FIG. 4N depicts representative ex vivo EGFP brain fluorescence images (left) and quantification (right). Scale bars, 0.5 inches.

FIG. 4O depicts representative ex vivo EGFP brain tissue fluorescence imaging and quantification post ICV injection. Scale bars, 0.5 inches. Data from WT animals were added for comparison only.

FIG. 4P depicts quantification of EGFP fluorescence in the brain, spleen, kidney, liver, heart, and skin (ear). Data for each condition is presented, from left to right, as Day 7 Saline, Day 7 ssASO1, Day 14 Saline, and Day 14 ssASO1. Mean±SEM are shown and two-way ANOVA with multiple comparisons Tukey's test was used, *p<0.0332, **p<0.0021. All scale bars, 0.5 inches.

FIGS. 4Q-4R depict representative fluorescence IHC brain images (FIG. 4Q) and quantification (FIG. 4R) of anti-ASO (red) and anti-EGFP (green) staining, as normalized to the total DAPI+ area per section. Eight or more randomly selected brain sections were analyzed and average values are represented (n=3). Scale bars, 1000 μm. For all panels, mean±SEM are shown, and one-way (b) or two-way (d, e, g) ANOVA with multiple comparisons Tukey's tests were used, *p<0.0332, **p<0.0021, ***p<0.0002.

FIGS. 4S-4V depict EGFP splice-switching activity and ASO-EGFP colocalization in multiple brain regions. FIG. 4S depicts representative fluorescence IHC images showing EGFP signal. FIG. 4T depicts quantification of percent EGFP area in multiple brain regions. Scale bars, 100 μm. FIG. 4U depicts representative fluorescence IHC images showing EGFP localization with ASO and ASO-EGFP. FIG. 4V depicts the colocalization quantification of ASO and EGFP fluorescence in multiple brain regions. Scale bars, 100 μm. For all data, saline and 100/200 μg ssASO1-dosed animals were analyzed 14 days post ICV injection. Three sections were analyzed within each brain region, and the resulting means were plotted for each treatment (n=3). For all panels, mean±SEM are shown and one-way ANOVA with multiple comparisons Tukey's test was used for each brain region analyzed, *p<0.05, **p<0.01, ***p<0.001, ****p<0.0001.

FIG. 5A depicts representative images of mKate2 (top) fluorescence expression in live saline or ssASO1-injected ssEGFP.HBB ki mice 14 days post ICV injection.

FIG. 5B depicts quantification of mKate2 fluorescence in the tail, back paw, front paw, nose, ear, and head. Data for each condition is presented, from left to right, as Saline, ssASO1 (100 μg), and ssASO1 (200 μg).

FIG. 5C depicts representative image of EGFP fluorescence expression in live saline or ssASO1-injected ssEGFP.HBB ki mice 14 days post intracerebroventricular injection (ICV) injection. Scale bars, 0.5 inches.

FIG. 5D depicts quantification of EGFP fluorescence in the tail, back paw, front paw, nose, skin (ear), and head. EGFP expression in the head (brain) trends higher in ssASO1-treated animals. Data for each condition is presented, from left to right, as Saline, ssASO1 (100 μg), and ssASO1 (200 μg).

FIG. 5E depicts quantification of ex vivo brain, spleen, kidney, liver, heart, and skin (ear) tissue weights from injected animals. Data for each condition is presented, from left to right, as Saline, ssASO1 (100 μg), and ssASO1 (200 μg).

FIG. 5F depicts representative ex vivo mKate2 (left) and EGFP (right) tissue fluorescence imaging 14 days following ssASO1 ICV injection.

FIG. 5G depicts quantification of mKate2 fluorescence in the brain, spleen, kidney, liver, heart, and skin (ear). Data for each condition is presented, from left to right, as Saline, ssASO1 (100 μg), and ssASO1 (200 μg).

FIGS. 511-5I depict representative ex vivo mKate2 (FIG. 511) and EGFP (FIG. 5I) brain tissue fluorescence imaging and quantification 14 days post ICV injection. Scale bars, 0.5 inches.

FIG. 5J depicts quantification of EGFP fluorescence in the brain, spleen, kidney, liver, heart, and skin (ear). Data for each condition is presented, from left to right, as Saline, ssASO1 (100 μg), and ssASO1 (200 μg). Mean±SEM are shown and one-way ANOVA with multiple comparisons Tukey's test was used, **p<0.0021, ***p<0.0002. All scale bars, 0.5 inches.

FIGS. 5K-5L depict representative fluorescence IHC brain images (FIG. 5K) and quantification (FIG. 5L) of anti-ASO (red) and anti-EGFP (green) staining, as normalized to the total DAPI+ area per section. Eight or more randomly selected brain sections were analyzed and average values are represented (n=3). Scale bars, 1000 μm. For all panels, mean±SEM are shown and one-way ANOVA with multiple comparisons Tukey's test was used, *p<0.0332, **p<0.0021, ***p<0.0002, ****p<0.0001.

FIGS. 6A-6D depict representative fluorescence IHC images showing EGFP localization with cell-type specific markers including NeuN (neurons, FIG. 5A), Iba1 (microglia, FIG. 5B), S100B (astrocytes, FIG. 5C), and Olig2 (oligodendrocytes, FIG. 5D) in unique brain regions. Colocalization (yellow) is depicted using white arrowheads (up to three shown). Scale bars, 100 μm.

FIGS. 6E-6H depict representative fluorescence IHC images showing EGFP localization with cell-type specific markers including NeuN (neurons, FIG. 6E), Iba1 (microglia, FIG. 6F), S100B (astrocytes, FIG. 6G), and Olig2 (oligodendrocytes, FIG. 6H) in unique brain regions. Scale bars, 200 μm.

FIG. 6I depicts mean Mander's coefficients quantifying the fraction of overlap/colocalization between EGFP and these cell-specific markers in the cortex and hippocampus, or corpus callosum (oligodendrocytes). Four images were acquired and analyzed within each brain region, and the resulting means were plotted for each treatment (n=3). For each brain region data is presented from left to right as, 100 μg ssASO1 Day 7, 100 μg ssASO1 Day 14, and 200 μg ssASO1 Day 14. For all panels, mean±SEM are shown and one-way ANOVA with multiple comparisons Tukey's test was used for each cell type within a single brain region, all comparisons were not significant. Non-edited images used for quantification are shown in FIGS. 6E-6H.

FIG. 6J depicts the percent EGFP+ neurons, microglia, and astrocytes in the cortex and hippocampus, or corpus callosum (oligodendrocytes) as measured using object overlap. Four images were acquired and analyzed within each brain region, and the resulting means were plotted for each treatment (n=3). Quantification from animals 14 days post saline injection is shown for comparison purposes only and was not used for statistical analysis. For each brain region data is presented from left to right as, Saline Day 14, 100 μg ssASO1 Day 7, 100 μg ssASO1 Day 14, and 200 μg ssASO1 Day 14. For all panels, mean±SEM are shown and one-way ANOVA with multiple comparisons Tukey's test was used for each cell type within a single brain region; *p<0.05 and **p<0.01.

FIG. 7A depicts representative images of mKate2.ssEGFP.HBB HEK293 cells treated with increasing concentrations of free ASO or ASO-LNPs for 3 days. Scale bar, 200 mm.

FIG. 7B depicts dose-response curves analyzing the percentage of EGFP+ HEK293 cells after 3 days of treatment. The experiment was completed in duplicate on three independent cell cultures for a total n=6. Signs of cell death were observed for formulations 3 and 4 when dosed at 500 nM. Mean±SEM are shown.

FIG. 8A depicts an experimental protocol where saline or pooled ASO-LNPs were administered i.v. in 1- to 3-month-old mixed-gender mKate2.ssEGFP.HBB ki mice via the tail vein (n=4/treatment) and EGFP splice-switching was evaluated over 20 days.

FIG. 8B depicts the percentage body weight change normalized to day 0 for mice treated with saline or ASO-LNP on day 6 or day 20 following administration.

FIG. 8C depicts representative ex vivo mKate2 (left) and EGFP (right) tissue fluorescence imaging acquired 6 and 20 days following i.v. injection of saline or ASO-LNPs in mice. Scale bar, 0.5 inches.

FIGS. 8D-8E depict quantification of mKate2 (FIG. 8D) and EGFP (FIG. 8E) fluorescence in the brain, spleen, kidneys, heart, liver, and lungs of treated mice. For each organ, data is displayed from left to right as Saline Day 6, ASO-LNPs Day 6, Saline Day 20, and ASO-LNP. For all panels, n=4 animals per treatment, mean±SEM are shown, and two-way ANOVA with multiple comparisons Tukey's tests were used; *p<0.05, **p<0.01, and ***p<0.001.

FIG. 8F depicts representative FACS plots of liver single-cell suspensions acquired from WT or ki mKate2.ssEGFP.HBB mice injected with saline or pooled ASO-LNPs.

FIG. 9A depicts the split Luc2 cassette (ssLuc2.HBB) structure. A human β-globulin (HBB) exon interrupts the Luc2 coding sequence, which is split into two regions. Splice-switching ASOs (ssASOs) block an aberrant 5′ splice site, restoring pre-mRNA splicing, and producing functional Luc2 protein. Note: depiction is not drawn to scale.

FIG. 9B depicts the construct used to test ASO-mediated Luc2 splicing correction in cells.

FIG. 9C depicts FACS data following transfection of the construct described in (FIG. 9B) into NLS-Cre expressing HEK293 cells and the isolation of pooled stable cells using serial FACS (mKate2+ sorting).

FIG. 9D depicts representative phase contrast and mKate2 fluorescence images of wild-type (wt, left) and pooled ssLuc2.HBB (right) HEK293 cells after 3 days in culture. Scale bars, 400 μm.

FIG. 9E depicts quantification of mKate2 fluorescence intensity in wt and ssLuc2.HBB cells. Unpaired two-tailed student's t-test, ****p<0.0001.

FIGS. 9F-9G depict representative images and dose-response curves analyzing Luc2 bioluminescence after treatment with increasing concentrations of ASO (FIG. 9F) or D-luciferin (FIG. 9G) after 3 days of treatment. D-luciferin concentration was maintained at 50 μM final for ASO dose-response (FIG. 9F), and ASO concentration was maintained at 500 nM for substrate (FIG. 9G) dose-response analysis. Not-treated (NT) wt and ssLuc2.HBB cells are shown for reference. The experiment was completed on four-independent cell cultures and mean±SEM are shown.

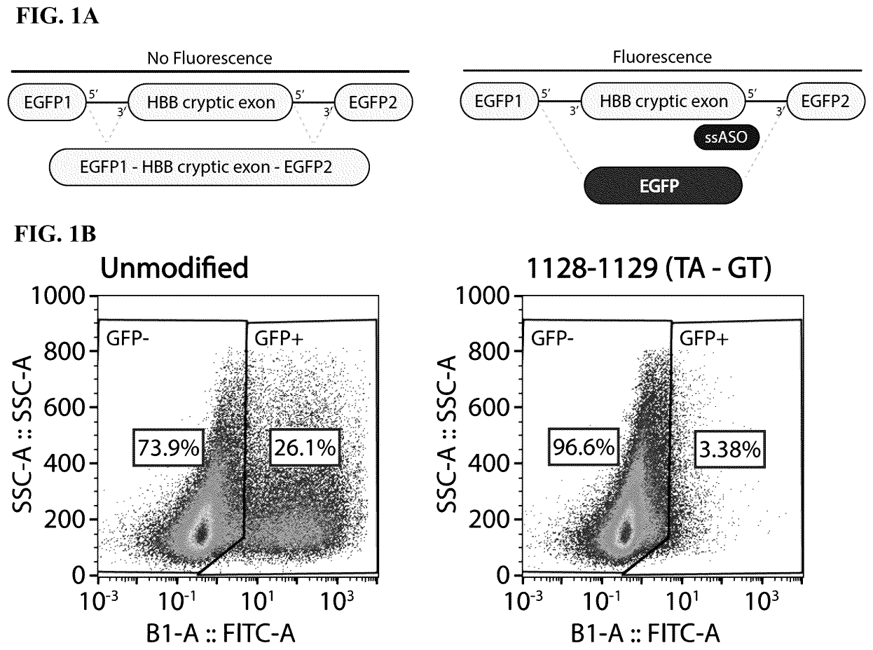

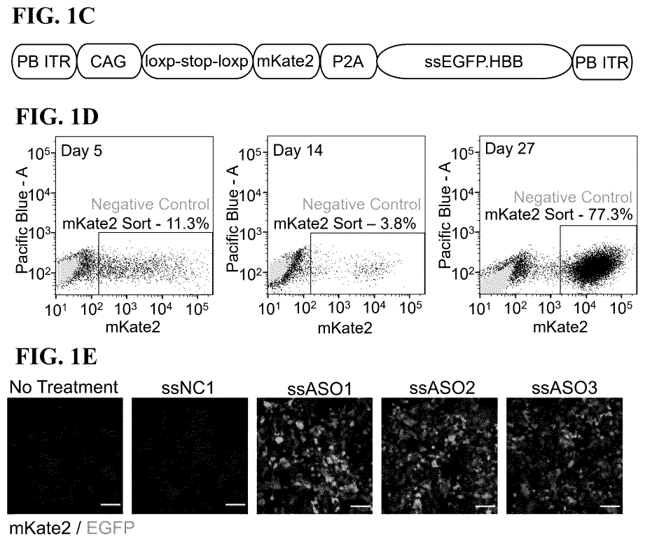

FIG. 9H depicts bioluminescence analysis of empty wells, wt cells, and mKate2.ssLuc2.HBB cells lacking ASO treatment. mKate2.ssLuc2.HBB cells show an increase in background bioluminescence after treatment with 50 μM D-luciferin, but this value is significantly lower than that observed in ASO-treated cells (see FIG. 1f).

FIGS. 9I-9J depict cellular mKate2 fluorescence intensity for ASO (FIG. 9I) and D-luciferin (FIG. 9J) dose-response studies (see FIG. 9F-9G, respectively, for bioluminescence analysis). No significant change in fluorescence was observed, suggesting ASO treatment does not affect construct expression or cell health. The experiment was completed on four-independent cell cultures.

FIG. 10A depicts the construct used to create a knock in (ki) mouse line expressing LSL.mKate2.ssLuc2.HBB. The transgene was inserted at the ROSA26 safe harbor locus and contains mKate2 followed by ssLuc2.HBB, which is driven by a CAG promoter. A loxP cassette was also included to ensure transgene expression is Cre-dependent.

FIG. 10B depicts a representative mouse crossing scheme. LSL.mKate2.ssLuc2.HBB mice were crossed with ROSA26.Cre.ki mice to generate animals expressing mKate2.ssLuc2.HBB throughout the entire body. Illustration was created using Biorender.com.

FIG. 10C depicts representative images of mKate2 fluorescence expression in live LSL.mKate2.ssLuc2.HBB, Cre.ki, and mKate2.ssLuc2.HBB animals.

FIG. 10D depicts quantification of mKate2 fluorescence in the tail, back paw, and front paw in loxP-stop-loxP (LSL) control mice (LSL.mKate2.ssLuc2.HBB), Cre knock-in control mice (Cre.Ki), and mKate2.ssLuc2.HBB mice. Data for each condition is presented, from left to right, as LSL.mKate2.ssLuc2.HBB, Cre.Ki, and mKate2.ssLuc2.HBB.

FIG. 10E depicts ex vivo brain, spleen, kidney, liver, heart, and skin (ear) tissue weights. Data for each condition is presented, from left to right, as LSL.mKate2.ssLuc2.HBB, Cre.Ki, and mKate2.ssLuc2.HBB.

FIG. 10F depicts representative images of mKate2 fluorescence in brain, spleen, kidney, liver, heart, and skin tissues isolated from loxP-stop-loxP (LSL) control mice (LSL.mKate2.ssLuc2.HBB, column 1), Cre knock-in control mice (Cre.Ki, column 2), and mKate2.ssLuc2.HBB animals (column 3).

FIG. 10G depicts quantification of mKate2 fluorescence intensity in tissues. Data for each condition is presented, from left to right, as LSL.mKate2.ssLuc2.HBB, Cre.Ki, and mKate2.ssLuc2.HBB. For all panels, fluorescence was measured using the same box size across each body part/tissue sample and n=2-3 animals per genotype. Mean±SEM are shown and one-way ANOVA with a Tukey's multiple comparisons test was used, *p<0.0332, **p<0.0021, ***p<0.0002, ****p<0.0001.

FIG. 10H depicts representative images of luciferase background expression in live Cre knock-in (Cre.ki) control, loxP-stop-loxP (LSL) control mice (LSL.mKate2.ssLuc2.HBB), and mKate2.ssLuc2.HBB animals 10 minutes after injection with 5 mg D-luciferin, and quantification (bottom left) of luciferase expression from live Cre.ki, LSL.mKate2.ssLuc2.HBB, and mKate2.ssLuc2.HBB animals in the tail, front paw, and back paw. Scale bar, 0.5 inches. For all panels, fluorescence or bioluminescence was measured using the same box size across each body part/tissue sample and n=3 animals per genotype. Mean±SEM are shown and one-way ANOVA with a Tukey's multiple comparisons test was used, *p<0.05, **p<0.01, ***p<0.0001, ****p<0.0001.

FIG. 11A illustrates a method comprising IV injecting 2-6 month old mKate2.ssLuc2.HBB mixed gender ki mice via the tail vein with a single dose of saline at day 42 (bottom row arrow) or doses of ssASO1 (25, 100, or 250 μg) on days 0, 1, 3, 7, 14, 21, 28, 35, or 42 (top row arrows), and measuring Luc2 splice-switching over the course of 6 weeks using bioluminescence as a readout.

FIG. 11B depicts percent body weight change normalized to Day 0 for animals treated with a single dose of saline or 9 doses of ssASO1 (25, 100, or 250 μg) over the course of 42 days.

FIG. 11C depicts representative images of Luc2 bioluminescence in the abdomen, head, and eyes of live animals 10 minutes after intraperitoneal (IP) injection with 5 mg D-luciferin. Animals treated with saline (Day 0) or ssASO1 (Day 1, 3, and 7) are shown for comparison. Scale bar, 0.5 inches.

FIGS. 11D-11F depict quantification of luciferase bioluminescence in the abdomen (FIG. 11D), head (FIG. 11E), and eyes (FIG. 11F) of live animals over the course of the experiment. Bioluminescence was measured in both eyes and average values were potted. The same box size was used to measure signal across each body part. For all panels, mean SEM are shown and n=6 animals/treatment.

FIGS. 11G-11I depict quantification of bioluminescence signal in the tail following IV (FIG. 11G), ICV (FIG. 11H), or IVT (FIG. 11I) injection of ssASO1 over the course of 42 days. The same box size was used to measure signal in the tail. For all panels, mean SEM are shown and n=6 animals/treatment.

FIG. 12A illustrates a method comprising ICV injecting 2-6 month old mKate2.ssLuc2.HBB mixed gender ki mice with a single dose of saline at day 42 (bottom row arrow) or doses of ssASO1 (12.5, 50, or 100 μg) on days 0, 1, 3, 7, 14, 21, 28, 35, or 42 (top row arrows), and measuring Luc2 splice-switching over the course of 6 weeks using bioluminescence as a readout.

FIG. 12B depicts acute toxicity score post injection for animals treated with a single dose of saline or 9 doses of ssASO1 administered via ICV injection over the course of 42 days.

FIG. 12C depicts the percent body weight change normalized to Day 0 for animals treated with a single dose of saline or 9 doses of ssASO1 administered via ICV injection over the course of 42 days.

FIG. 12D depicts representative images of Luc2 bioluminescence in the abdomen, head, and eyes of live animals 10 minutes after IP injection with 5 mg D-luciferin. Animals treated with saline (Day 0) or ssASO1 (Day 1, 3, and 7) are shown for comparison. Scale bar, 0.5 inches.

FIGS. 12E-12G depict quantification of bioluminescence signal in the abdomen (FIG. 12E), head (FIG. 12F), and eyes (FIG. 12G) of live animals over the course of the experiment. Bioluminescence was measured in both eyes and average values were potted. The same box size was used to measure signal across each body part. For all panels, mean SEM are shown and n=6 animals/treatment.

FIG. 13A illustrates a method comprising IVT injecting 2-6 month old mKate2.ssLuc2.HBB mixed gender ki mice in both eyes with a single dose of saline at day 42 (bottom row arrow) or doses of ssASO1 (6.25, 25, or 50 μg) on days 0, 1, 3, 7, 14, 21, 28, 35, or 42 (top row arrows), and measuring Luc2 splice-switching over the course of 6 weeks using bioluminescence as a readout.

FIG. 13B depicts percent body weight change normalized to Day 0 for animals treated with a single dose of saline or 9 doses of ssASO1 administered via IVT injection over the course of 42 days.

FIG. 13C depicts representative images of Luc2 bioluminescence in the abdomen, head, and eyes of live animals 10 minutes after IP injection with 5 mg D-luciferin. Animals treated with saline (Day 0) or ssASO1 (Day 1, 3, and 7) are shown for comparison. Scale bar, 0.5 inches.

FIGS. 13D-13F depict quantification of bioluminescence signal in the abdomen (FIG. 13D), head (FIG. 13E), and eyes (FIG. 13F) of live animals over the course of the experiment. Bioluminescence was measured in both eyes and average values were potted. The same box size was used to measure signal across each body part. For all panels, mean SEM are shown and n=6 animals/treatment.

### DETAILED DESCRIPTION

Provided herein are compositions, systems, and methods of assessing a feature of an ASO using a reporter protein. In some embodiments, a pre-mRNA encoding the reporter protein comprises a cryptic sequence that is regulated by a splice switching ASO to be assessed. Thus, beneficially, the present invention provides a fast and reliable method, system, and composition for screening activity of splice-switching oligonucleotides in cell culture or in vivo, in a transgenic mouse. In some embodiments, the cryptic sequence contains one or more mutations that reduces background splicing in the absence of ASO, which reduces “leakiness” and allows sensitive and reproducible detection of ASO activity.

### I. Methods of Assessing ASO Activity

In some embodiments, provided herein is a method of assessing delivery and/or splice-switching activity of an antisense oligonucleotide (ASO) comprising (i) providing a transgenic mouse comprising a plurality of cells which comprise a genetic sequence that is transcribed as a pre-mRNA which comprises a first segment encoding a first portion of a reporter protein, a cryptic sequence, and a second segment encoding a second portion of the reporter protein, (ii) introducing the ASO to the transgenic mouse, wherein the ASO alters splicing of the pre-mRNA to exclude the cryptic sequence from a mature mRNA transcript, thereby causing expression of a functional form of the reporter protein, and wherein the cryptic sequence comprises a sequence of SEQ ID NO.: 1 comprising nucleotide substitutions of T657G and A658T which result in decreased exclusion of the cryptic sequence in the absence of the ASO when compared to exclusion of the cryptic gene without the nucleotide substitutions of T657G and A658T; and (iii) detecting the expression of the reporter protein to assess the delivery and/or splice-switching activity of the ASO in the plurality of cells.

In some embodiments, provided herein is a method of assessing delivery and/or splice-switching activity of an antisense oligonucleotide (ASO) comprising: (i) providing a plurality of cultured cells, the cultured cells isolated from a transgenic animal comprising a genetic sequence that is transcribed as a pre-mRNA which comprises a first segment encoding a first portion of a reporter protein, a cryptic sequence, and a second segment encoding a second portion of the reporter protein, and (ii) introducing the ASO to the plurality of cultured cells, wherein the ASO alters splicing of the pre-mRNA to exclude the cryptic sequence from a mature mRNA transcript, thereby causing expression of a functional form of the reporter protein, and wherein the cryptic sequence comprises a sequence of SEQ ID NO.: 1 comprising nucleotide substitutions of T657G and A658T which result in decreased exclusion of the cryptic sequence in the absence of the ASO when compared to exclusion of the cryptic gene without the nucleotide substitutions of T657G and A658T, and (iii) detecting the expression of the reporter protein to assess the delivery and/or activity of the ASO in the plurality of cultured cells.

In some embodiments, provided herein is a method of assessing a feature of an antisense oligonucleotide (ASO) in a cell comprising: (i) providing the ASO to the cell, wherein the cell comprises a genetic sequence that is transcribed as a pre-mRNA which comprises a first segment encoding a first portion of a reporter protein, a cryptic sequence, and a second segment encoding a second portion of the reporter protein, and wherein the ASO alters splicing of the pre-mRNA to exclude the cryptic sequence from a mature mRNA transcript, thereby causing expression of the a functional form of the reporter protein, and wherein the cryptic sequence comprises one or more nucleotide insertions, deletions, or substitutions that result in decreased exclusion of the cryptic sequence in the absence of the ASO when compared to exclusion of the cryptic gene without the one or more nucleotide insertions, deletions, or substitutions, and (ii) detecting the expression of the reporter protein to assess a feature of the ASO in the cell. In some embodiments, the cryptic sequence is a cryptic β-globin (HBB) exon. In some embodiments, the cryptic HBB exon comprises nucleotide substitutions to reduce background expression of a reporter protein. In some embodiments, the cryptic HBB exon comprises a sequence of SEQ ID NO.: 1 comprising nucleotide substitutions of T657G and A658T which result in decreased exclusion of the cryptic sequence from mature mRNA transcripts in the absence of a bound ASO when compared to exclusion of the cryptic gene from mature mRNA transcripts without the nucleotide substitutions of T657G and A658T.

In some embodiments, the pre-mRNA construct comprises, from N-terminus to C-terminus, a first segment encoding a first portion of an EGFP reporter protein, a cryptic sequence, and a second segment encoding a second portion of the EGFP reporter protein, wherein binding of an ASO to the pre-mRNA excludes the cryptic sequence from a mature EGFP mRNA transcript, thereby causing expression of a functional form of the EGFP reporter protein, and wherein the pre-mRNA comprises a sequence of SEQ ID NO.: 21 comprising nucleotide substitutions of T1128G and A1129T which result in decreased exclusion of the cryptic sequence in the absence of the ASO when compared to exclusion of the cryptic gene without the nucleotide substitutions of T1128G and A1129T.

In some embodiments, the pre-mRNA construct comprises, from N-terminus to C-terminus, a first segment encoding a first portion of a luciferase reporter protein, a cryptic sequence, and a second segment encoding a second portion of the luciferase reporter protein, wherein binding of an ASO to the pre-mRNA excludes the cryptic sequence from a mature luciferase mRNA transcript, thereby causing expression of a functional form of the luciferase reporter protein, and wherein the pre-mRNA comprises a sequence of SEQ ID NO.: 22 comprising nucleotide substitutions of T2001G and A2002T which result in decreased exclusion of the cryptic sequence in the absence of the ASO when compared to exclusion of the cryptic gene without the nucleotide substitutions of T2001G and A2002T.

### A. Splice Switching ASOs

In some embodiments, provided herein is a method of assessing delivery and/or splice-switching activity of an antisense oligonucleotide (ASO). In some embodiments, the ASO is comprised of DNA or RNA. In some embodiments, the ASO comprises one or more modifications to improve the half-life and stability of the ASO. In some embodiments, a modification to the ASO comprises a modified sugar moiety and/or a modified nucleobase and/or a modified internucleoside linkage.

In some embodiments, provided herein is a method comprising the delivery of a plurality of ASOs to a transgenic mouse or cells in culture, such as those isolated from a transgenic mouse. In some embodiments, the amount of ASO that is introduced to the transgenic mouse is in the range of about 1 μg and about 200 μg and any amount within the range. In some embodiments, the amount of ASO that is introduced to the transgenic mouse is about 50 μg or more. In some embodiments, the amount of ASO that is introduced to the transgenic mouse is about 100 μg or more. In some embodiments, the amount of ASO that is introduced to the transgenic mouse is at least 1 μg. In some embodiments, the amount of ASO that is introduced to the transgenic mouse is at least 10 μg. In some embodiments, the amount of ASO that is introduced to the transgenic mouse is at least 50 μg. In some embodiments, the amount of ASO that is introduced to the transgenic mouse is at least 100 μg.

In some embodiments, the amount of ASO delivered to the transgenic mouse is measured as a molar concentration. In some embodiments, the concentration of ASO that is introduced to the transgenic mouse is in the range of about 0.5 μM and about 30 μM and any amount within the range. In some embodiments, the concentration of ASO that is introduced to the transgenic mouse is at least about 0.5 μM, about 1.0 μM, about 2.0 μM, about, 3.0 μM, about 4.0 μM, about 5.0 μM, about 10.0 μM, about 15.0 μM, about 20.0 μM, about 25.0 μM, or about 30.0 μM. In some embodiments, the concentration of ASO that is introduced to the transgenic mouse is greater than 5.0 μM. In some embodiments, the concentration of ASO that is introduced to the transgenic mouse is about 0.5 μM or more. In some embodiments, the concentration of ASO that is introduced to the transgenic mouse is about 1.0 μM or more. In some embodiments, the concentration of ASO that is introduced to the transgenic mouse is about 2.0 μM or more. In some embodiments, the concentration of ASO that is introduced to the transgenic mouse is about 3.0 μM or more. In some embodiments, the concentration of ASO that is introduced to the transgenic mouse is about 4.0 μM or more. In some embodiments, the concentration of ASO that is introduced to the transgenic mouse is about 5.0 μM or more. In some embodiments, the concentration of ASO that is introduced to the transgenic mouse is about 10.0 μM or more. In some embodiments, the concentration of ASO that is introduced to the transgenic mouse is about 15.0 μM or more. In some embodiments, the concentration of ASO that is introduced to the transgenic mouse is about 20.0 μM or more. In some embodiments, the concentration of ASO that is introduced to the transgenic mouse is about 25.0 μM or more. In some embodiments, the concentration of ASO that is introduced to the transgenic mouse is about 30.0 μM or more.

In some embodiments, provided herein is a method comprising the delivery of a plurality of ASOs to an organ or tissue in an ex vivo setting wherein the organ or tissue is isolated from a transgenic animal such as mouse. In some embodiments, the amount of ASO that is introduced to the organ or tissue is in the range of about 1 μg and about 200 μg and any amount within the range. In some embodiments, the amount of ASO that is introduced to the organ or tissue is about 50 μg or more. In some embodiments, the amount of ASO that is introduced to the organ or tissue is about 100 μg or more. In some embodiments, the amount of ASO that is introduced to the organ or tissue is at least 1 μg. In some embodiments, the amount of ASO that is introduced to the organ or tissue is at least 10 μg. In some embodiments, the amount of ASO that is introduced to the organ or tissue is at least 50 μg. In some embodiments, the amount of ASO that is introduced to the organ or tissue is at least 100 μg.

In some embodiments, the amount of ASO delivered to the organ or tissue is measured as a molar concentration. In some embodiments, the concentration of ASO that is introduced to the organ or tissue is in the range of about 0.5 μM and about 5.0 μM and any amount within the range. In some embodiments, the concentration of ASO that is introduced to the organ or tissue is at least about 0.5 μM, about 1.0 μM, about 2.0 μM, about, 3.0 μM, about 4.0 μM, about 5.0 μM, about 10.0 μM, about 15.0 μM, about 20.0 μM, about 25.0 μM, or about 30.0 μM. In some embodiments, the concentration of ASO that is introduced to the organ or tissue is greater than 5.0 μM. In some embodiments, the concentration of ASO that is introduced to the organ or tissue is about 0.5 μM or more. In some embodiments, the concentration of ASO that is introduced to the organ or tissue is about 1.0 μM or more. In some embodiments, the concentration of ASO that is introduced to the organ or tissue is about 2.0 μM or more. In some embodiments, the concentration of ASO that is introduced to the organ or tissue is about 3.0 μM or more. In some embodiments, the concentration of ASO that is introduced to the organ or tissue is about 4.0 μM or more. In some embodiments, the concentration of ASO that is introduced to the organ or tissue is about 5.0 μM or more. In some embodiments, the concentration of ASO that is introduced to the organ or tissue is about 10.0 μM or more. In some embodiments, the concentration of ASO that is introduced to the organ or tissue is about 15.0 μM or more. In some embodiments, the concentration of ASO that is introduced to the organ or tissue is about 20.0 μM or more. In some embodiments, the concentration of ASO that is introduced to the organ or tissue is about 25.0 M or more. In some embodiments, the concentration of ASO that is introduced to the organ or tissue is about 30.0 μM or more.

In some embodiments, provided herein is a method comprising the delivery of a plurality of ASOs to a plurality of cultured cells in an in vitro setting. In some embodiments, the plurality of cultured cells are isolated from the transgenic mouse. In some embodiments, the amount of ASO that is introduced to the plurality of cultured cells is in the range of about 1 μg and about 200 μg and any amount within the range. In some embodiments, the amount of ASO that is introduced to the plurality of cultured cells is about 50 μg or more. In some embodiments, the amount of ASO that is introduced to the plurality of cultured cells is about 100 μg or more. In some embodiments, the amount of ASO that is introduced to the plurality of cultured cells is at least 1 μg. In some embodiments, the amount of ASO that is introduced to the plurality of cultured cells is at least 10 μg. In some embodiments, the amount of ASO that is introduced to the plurality of cultured cells is at least 50 μg. In some embodiments, the amount of ASO that is introduced to the plurality of cultured cells is at least 100 μg.

In some embodiments, the amount of ASO delivered to the plurality of cultured cells is measured as a molar concentration. In some embodiments, the concentration of ASO that is introduced to the plurality of cultured cells is in the range of about 0.5 μM and about 5.0 μM and any concentration within the range. In some embodiments, the concentration of ASO that is introduced to the plurality of cultured cells is at least about 0.5 μM, about 1.0 μM, about 2.0 μM, about, 3.0 μM, about 4.0 μM, about 5.0 μM, about 10.0 μM, about 15.0 M, about 20.0 μM, about 25.0 μM, or about 30.0 μM. In some embodiments, the concentration of ASO that is introduced to the plurality of cultured cells is greater than 5.0 μM. In some embodiments, the concentration of ASO that is introduced to the plurality of cultured cells is about 0.5 μM or more. In some embodiments, the concentration of ASO that is introduced to the plurality of cultured cells is about 1.0 μM or more. In some embodiments, the concentration of ASO that is introduced to the plurality of cultured cells is about 2.0 μM or more. In some embodiments, the concentration of ASO that is introduced to the plurality of cultured cells is about 3.0 μM or more. In some embodiments, the concentration of ASO that is introduced to the plurality of cultured cells is about 4.0 μM or more. In some embodiments, the concentration of ASO that is introduced to the plurality of cultured cells is about 5.0 μM or more. In some embodiments, the concentration of ASO that is introduced to the plurality of cultured cells is about 10.0 μM or more. In some embodiments, the concentration of ASO that is introduced to the plurality of cultured cells is about 15.0 μM or more. In some embodiments, the concentration of ASO that is introduced to the plurality of cultured cells is about 20.0 μM or more. In some embodiments, the concentration of ASO that is introduced to the plurality of cultured cells is about 25.0 μM or more. In some embodiments, the concentration of ASO that is introduced to the plurality of cultured cells is about 30.0 μM or more.

In some embodiments, the plurality of ASOs are about 12 to about 30 nucleotides in length. In some embodiments, the plurality of ASOs delivered to the transgenic mouse are about 14 to about 18 nucleotides in length. In some embodiments, the plurality of ASOs are about 16 nucleotides in length. In some embodiments, the plurality of ASOs are 14, 15, 16, 17, or 18 nucleotides in length

In some embodiments, the plurality of ASOs comprise the same nucleotide sequence. In some embodiments, the nucleotide sequence of the ASO is set forth in SEQ ID NOs: 17-20. In some embodiments, the plurality of ASOs comprise different nucleotide sequences from each other.

In some embodiments, the sequence of the plurality of ASOs delivered to the transgenic mouse or cultured cells is complementary to a part of the cryptic sequence. In some embodiments, the sequence of the plurality of ASOs is complementary to a splice donor site or a splice acceptor site that regulates splicing of the cryptic sequence. In some embodiments, binding of the ASO to the cryptic sequence results in a splice switching event. In some embodiments, the ASO prevents splicing machinery (e.g., protein splicing factors) from recognizing the cryptic exon, such as the HBB exon. In some embodiments, binding of the ASO to the cryptic sequence results in altered protein expression. In some embodiments, binding of the ASO to the cryptic sequence results in altered expression of a reporter protein. In some embodiments, the ASO is capable of preventing inclusion of a cryptic HBB exon into a mature mRNA. In some embodiments, binding of the ASO to the cryptic sequence results in increased expression of the reporter protein. In some embodiments, binding of the ASO to the cryptic sequence results in exclusion of a cryptic exon from an mRNA transcript. In some embodiments, binding of the ASO to the cryptic sequence results in an increase in fluorescence of the reporter protein.

In some embodiments, the ASO is capable of binding to the splice site within the cryptic HBB exon. In some embodiments, the ASO is capable of binding within the genetic sequence that is transcribed as a pre-mRNA encoding the reporter protein. In some embodiments, targeting the splice site on the pre-mRNA encoding the activity reporter makes the splice site less available for splicing. In some embodiments, the splice site may be a 5′-splice site (i.e., donor splice site) or a 3′ splice site (i.e., acceptor splice site).

The term “complementarity” describes the capacity for Watson-Crick base-pairing of nucleosides/nucleotides. Watson-Crick base pairs are guanine (G)-cytosine (C) and adenine (A)—thymine (T)/uracil (U). It will be understood that oligonucleotides may comprise nucleosides with modified nucleobases, for example 5-methyl cytosine is often used in place of cytosine, and as such the term complementarity encompasses Watson Crick base-pairing between non-modified and modified nucleobases (see for example Hirao et al (2012) Accounts of Chemical Research vol 45 page 2055 and Bergstrom (2009) Current Protocols in Nucleic Acid Chemistry Suppl. 37 1.4.1).

The term “% complementary” as used herein, refers to the proportion of nucleotides (in percent) of a contiguous nucleotide sequence in a nucleic acid molecule (e.g. oligonucleotide) which, across the contiguous nucleotide sequence, are complementary to a reference sequence (e.g. a target sequence or sequence motif). The percentage of complementarity is thus calculated by counting the number of aligned nucleobases that are complementary (from Watson Crick base pairs) between the two sequences (when aligned with the target sequence 5′-3′ and the oligonucleotide sequence from 3′-5′), dividing that number by the total number of nucleotides in the oligonucleotide and multiplying by 100. In such a comparison a nucleobase/nucleotide which does not align (form a base pair) is termed a mismatch. Insertions and deletions are not allowed in the calculation of % complementarity of a contiguous nucleotide sequence. It will be understood that in determining complementarity, chemical modifications of the nucleobases are disregarded as long as the functional capacity of the nucleobase to form Watson Crick base pairing is retained (e.g. 5′-methyl cytosine is considered identical to a cytosine for the purpose of calculating % identity).

In some embodiments, provided herein is a method of assessing delivery and/or splice-switching activity of an antisense oligonucleotide (ASO); wherein the ASO comprises one or more chemical modifications. In some embodiments, the one or more chemical modifications comprise a modification to the phosphate backbone and/or a modification to the sugar. In some embodiments, the one or more chemical modifications comprise 2-MOE, 2′-OMe, LNA, GalNAc, 5′ methylcytosine, a phosphorothioate bond, a phosphorodithioate bond, an alkylphosphonate bond, a phosphoroamidate bond, a boranophosphate bond, and/or a morpholino ring.

The term, “nucleoside” as used herein refers to a purine or pyrimidine base and a ribose or a deoxyribose sugar connected via a β-glycosidic linkage.

The term “modified nucleoside” or “nucleoside modification” as used herein refers to nucleosides modified as compared to the equivalent DNA or RNA nucleoside by the introduction of one or more modifications of the sugar moiety or the (nucleo)base moiety. In some embodiments, one or more of the modified nucleosides of the antisense oligonucleotides of the invention comprise a modified sugar moiety. The term modified nucleoside may also be used herein interchangeably with the term “nucleoside analogue” or modified “units” or modified “monomers”. Nucleosides with an unmodified DNA or RNA sugar moiety are termed DNA or RNA nucleosides herein. Nucleosides with modifications in the base region of the DNA or RNA nucleoside are still generally termed DNA or RNA if they allow Watson Crick base pairing. Exemplary modified nucleosides which may be used in the compounds of certain embodiments include LNA, 2′-0-MO E and morpholino nucleoside analogues. In some embodiments, the ASO described herein may comprise one or more modified nucleosides. In some embodiments, the modified nucleoside is a 2′ sugar modified nucleoside. In some embodiments, a 2′ sugar modified nucleoside is a nucleoside which has a substituent other than H or —OH at the 2′ position (2′ substituted nucleoside) or comprises a 2′ linked biradicle capable of forming a bridge between the 2′ carbon and a second carbon in the ribose ring, such as LNA (2′-4′ biradicle bridged) nucleosides. In some embodiments, the 2′ sugar modified nucleoside is independently selected from the group consisting of 2′-O-alkyl-RNA; 2′-O-methyl RNA (2′-OMe); 2′-alkoxy-RNA; 2′-O-methoxyethyl-RNA (2′-MOE); 2′-amino-DNA; 2′-fluro-RNA; 2′-fluoro-DNA; arabino nucleic acid (ANA); 2′-fluoro-ANA; locked nucleic acid (LNA), and any combination thereof. In some embodiments, such modifications include those where the ribose ring structure is modified, e.g. by replacement with a hexose ring (HNA), or a bicyclic ring, which typically have a biradical bridge between the C2 and C4 carbons on the ribose ring (LNA), or an unlinked ribose ring which typically lacks a bond between the C2 and C3 carbons (e.g. UNA). Modified nucleosides also include nucleosides where the sugar moiety is replaced with a non-sugar moiety, for example in the case of peptide nucleic acids (PNA), or morpholino nucleic acids.

In some embodiments, the modified nucleoside is a 2′ sugar modified nucleoside, such as a 2′ sugar modified nucleoside independently selected from the group consisting of 2′-O-alkyl-RNA; 2′-O-methyl RNA (2′-OMe); 2′-alkoxy-RNA; 2′-O-methoxyethyl-RNA (2′-MOE); 2′-amino-DNA; 2′-fluro-RNA; 2′-fluoro-DNA; arabino nucleic acid (ANA); 2′-fluoro-ANA; locked nucleic acid (LNA), and any combination thereof

In some embodiments, the modified nucleoside may comprise an LNA nucleoside. In some embodiments, the LNA nucleoside may comprise an LNA nucleoside selected from the group consisting of constrained ethyl nucleoside (cEt), or β-D-oxy-LNA. In some embodiments, the antisense oligonucleotide of the invention may comprise LNA nucleosides and non-LNA nucleosides, such as DNA nucleosides. In some embodiments, the antisense oligonucleotide, or contiguous nucleotide sequence thereof, may comprise LNA and DNA nucleosides. In some embodiments, all of the nucleosides of the antisense oligonucleotide, or contiguous nucleotide sequence thereof, are independently selected from LNA and DNA nucleosides. In some embodiments, the length of contiguous DNA nucleosides present within the antisense oligonucleotide, or contiguous nucleotide sequence thereof, is limited so as to prevent RNaseH recruitment which results in target RNA degradation. In some embodiments, suitably the antisense oligonucleotide or contiguous nucleotide sequence thereof does not comprise more than four contiguous DNA nucleosides, more advantageously does not comprise more than 3 contiguous DNA nucleosides.

The term “modified internucleoside linkage” is defined as generally understood by a skilled person as linkages other than phosphodiester (PO) linkages that covalently couple two nucleosides together. The oligonucleotides of the invention in some embodiments may therefore comprise one or more modified internucleoside linkages such as one or more phosphorothioate internucleoside linkage.

In some embodiments at least 50% of the internucleoside linkages in the oligonucleotide, or contiguous nucleotide sequence thereof, are phosphorothioate, such as at least 60%, such as at least 70%, such as at least 75%, such as at least 80% or such as at least 90% or more of the internucleoside linkages in the oligonucleotide, or contiguous nucleotide sequence thereof, are phosphorothioate. In some embodiments all of the internucleoside linkages of the oligonucleotide, or contiguous nucleotide sequence thereof, are phosphorothioate. In some embodiments, all the internucleoside linkages of the contiguous nucleotide sequence of the oligonucleotide are phosphorothioate, or all the internucleoside linkages of the oligonucleotide are phosphorothioate linkages.

In some embodiments, the ASO may comprise one or more modified internucleoside linkages, such as one or more phosphorothioate internucleoside linkages. In some embodiments, a modified internucleoside linkage is defined as linkages other than phosphodiester (PO) linkages, that covalently couple two nucleosides together. In some embodiments, a majority of the internucleoside linkages within the ASO are phosphorothioate or boranophosphate internucleoside linkages. In some embodiments, at least 75% the internucleoside linkages within the ASO are phosphorothioate or boranophosphate internucleoside linkages. In some embodiments all the internucleotide linkages in the contiguous sequence of the oligonucleotide are phosphorothioate linkages.

The term nucleobase includes the purine (e.g. adenine and guanine) and pyrimidine (e.g. uracil, thymine and cytosine) moiety present in nucleosides and nucleotides which form hydrogen bonds in nucleic acid hybridization. In the context of the present invention, at least in some embodiments, the term nucleobase also encompasses modified nucleobases which may differ from naturally occurring nucleobases, but which are functional during nucleic acid hybridization. In this context “nucleobase” refers to both naturally occurring nucleobases such as adenine, guanine, cytosine, thymidine, uracil, xanthine and hypoxanthine, as well as non-naturally occurring variants. Such variants are for example described in Hirao et al (2012) Accounts of Chemical Research vol 45 page 2055 and Bergstrom (2009) Current Protocols in Nucleic Acid Chemistry Suppl. 37 1.4.1.

In some embodiments, the nucleobase moiety is modified by changing the purine or pyrimidine into a modified purine or pyrimidine, such as substituted purine or substituted pyrimidine, such as a nucleobase selected from isocytosine, pseudoisocytosine, 5-methyl cytosine, 5-thiozolo-cytosine, 5-propynyl-cytosine, 5-propynyl-uracil, 5-bromouracil 5-thiazolo-uracil, 2-thio-uracil, 2′thio-thymine, inosine, diaminopurine, 6-aminopurine, 2-aminopurine, 2,6-diaminopurine and 2-chloro-6-aminopurine, 5′ nitroindole.

The nucleobase moieties may be indicated by the letter code for each corresponding nucleobase, e.g. A, T, G, C or U, wherein each letter may optionally include modified nucleobases of equivalent function. For example, in the exemplified oligonucleotides, the nucleobase moieties are selected from A, T, G, C, and 5-methyl cytosine. Optionally, for LNA gapmers, 5-methyl cytosine LNA nucleosides may be used.

In some embodiments, the ASO comprises modifications to promote splice modulation and reduce the recruitment of RNaseH. In some embodiments, the ASO does not comprise a region of more than 3 or more than 4 contiguous DNA nucleosides to reduce the recruitment of RNaseH. In some embodiments, the ASO comprises a mixmer design, comprising sugar modified nucleosides, such as 2′ sugar modified nucleosides, and short regions of DNA nucleosides, such as 1, 2 or 3 DNA nucleosides. In some embodiments, the mixmer design alternates between 1 LNA and 1 DNA nucleoside, e.g. LDLDLDLDLDLDLDL with 5′ and 3′ terminal LNA nucleosides. In some embodiments, the mixmer design alternates every third nucleoside, e.g. LDDLDDLDDLDDLDDL, where every third nucleoside is a LNA nucleoside.

In some embodiments, the ASO comprises a totalmer design, wherein the ASO does not comprise DNA or RNA nucleosides. In some embodiments, the ASO comprises only 2′-O-MOE nucleosides, such as a fully MOE phosphorothioate, e.g. MMMMMMMMMIMMMMMMMMMMMM, where M=2′-O-MOE, which are reported to be effective splice modulators for therapeutic use. In some embodiments, the totalmer may comprise a mixture of modified nucleosides, such as MLMLMLMLMLMLMLMLMLML, wherein L=LNA and M=a non LNA modified nucleoside such as a 2′-O-MOE nucleosides.

In some embodiments, provided herein is a method of assessing delivery and/or splice-switching activity of an antisense oligonucleotide (ASO); wherein the ASO is formulated for direct delivery to an organ, tissue or cell in the transgenic mouse or in tissues or cells isolated from the transgenic mouse. In some embodiments, the formulation of the plurality of ASOs may be affected by many parameters, including, but not limited to, the formulation composition, nature of particle, degree of loading, polynucleotide (e.g., ASO) to lipid/lipidoid ratio, nature of polynucleotides (e.g., ASO) such as sequence contents, single-stranded or double-stranded, linear or circular, length and modifications, particle sizes and charges, and administration routes, etc.

In some embodiments, the formulation may comprise a pharmaceutically acceptable excipient, which, as used herein, includes, but is not limited to, any and all solvents including water, dispersion media, diluents, or other liquid vehicles, dispersion or suspension aids, surface active agents, isotonic agents, a solubilizing agent, a tonicity agent, a pH adjuster, a buffering agent, thickening or emulsifying agents, preservatives, and the like, as suited to the particular dosage form desired. Various excipients for formulating pharmaceutical compositions and techniques for preparing the composition are known in the art (see Remington: The Science and Practice of Pharmacy, 21st Edition, A. R. Gennaro, Lippincott, Williams & Wilkins, Baltimore, MD, 2006; incorporated herein by reference in its entirety). The use of a conventional excipient medium may be contemplated within the scope of the present disclosure, except insofar as any conventional excipient medium may be incompatible with a substance or its derivatives, such as by producing any undesirable biological effect or otherwise interacting in a deleterious manner with any other component(s) of the pharmaceutical composition. In some embodiments, the one or more excipients may increase the stability of the antisense polynucleotide (e.g., ASO); increase cell penetration; permit the sustained, controlled or delayed release; or alter the biodistribution (e.g., target the plurality of ASOs to specific tissues or cell types).

In some embodiments, the plurality of ASOs described herein are formulated and prepared by any method known or hereafter developed in the art of pharmacology. In general, such preparatory methods include the step of bringing the active ingredient (e.g., antisense polynucleotide) into association with an excipient and/or one or more other accessory ingredients, and then, if necessary and/or desirable, dividing, shaping and/or packaging the plurality of ASOs into a desired single- or multi-dose unit. In some embodiments, the plurality of ASOs

In some embodiments, the plurality of ASOs may be formulated with alternatives excipients such as lipids, lipidoids, liposomes, lipid nanoparticles, polymers, lipoplexes, core-shell nanoparticles, peptides, proteins, nucleic acid molecules, cells, organelles, explants, nanoparticle mimics and combinations thereof.

In some embodiments, the formulation of the plurality of ASOs comprises a pharmaceutically acceptable carrier. In some embodiments, the pharmaceutically acceptable carrier comprises complexes, micelles, liposomes or particles. Such carrier compositions can be prepared containing any suitable lipids and lipidoids and therefore, can result in an effective delivery of the plurality of ASOs following the injection of a formulation via localized and/or systemic routes of administration.

In some embodiments, the plurality of ASOs and compositions of the present disclosure may be formulated using one or more lipids and/or lipidoids. As used herein, the term “lipidoid” refers to any material having characteristics of a lipid. Lipidoids can be lipid-like structures containing multiple secondary and tertiary amine functionalities, which confer highly efficient interaction with nucleic acid molecules.

The synthesis of lipids and lipidoids has been extensively discussed and formulations containing the lipids and lipidoids are particularly suitable for delivery of nucleic acids. Use of the lipids and lipidoids to formulate and effectively deliver double stranded small RNAs (siRNAs), single stranded mRNAs and gene therapy has been described in mice and non-human primates (e.g., Lvins et al., 2010); Akinc et al., Nat Biotechnol. 2008 26:561-569; Love et al., Proc Natl Acad Sci USA. 2010, 107:1864-1869; Siegwart et al., Proc Natl Acad Sci USA. 2011, 108: 12996-3001; Leuschner et al., Nat Biotechnol. 2011, 29: 1005-1010; Roberts et al., Methods Mol. Biol., 2016, 1364:2991-310; Ball et al. Nato. Lett. 2018, 18(6):3814-3822; Lokras et al., Methods Mol. Biol., 2021, 2282: 137-157; Schrom et al., A7 Z. Ther. Nucleic Acids, 2017, 7:350-365; the contents of all of which are incorporated herein by references in their entirety).

The lipids and lipidoids can be cationic lipids and lipidoids. Cationic lipids typically feature a positively charged head group followed by hydrophobic tails of varying compositions, wherein the head and tail are connected by a linker, such as an ether, ester or amide. Without wishing to be bound by any theory, their cationic head groups neutralize the anionic charges of the nucleic acids that they transport.

In some embodiments, the lipids may comprise ionizable cationic lipids, anionic lipids, neutral lipids, or any combination thereof. In some embodiments, ionizable lipids such as Dlin-MC3-DMA (MC3), Dlin-KC2-DMA (KC2), and cKK-E12 may be used for the packaging of the plurality of ASOs.

In some embodiments, the plurality of ASOs described herein may be formulated using one or more polymers, or polymer containing nanoparticles (NPs). In some embodiments, the polymer may be biocompatible and biodegradable. The physicochemical properties of polymers (e.g., composition, molecular weight, and polydispersity) can be modified to achieve specialized formulations for nucleic acid delivery. Polymers may be naturally derived or synthetic. In some embodiments, the polymers used in the present disclosure have undergone processing to reduce and/or inhibit the attachment of unwanted substances such as, but not limited to, bacteria, to the surface of the polymer. The polymer may be processed by methods known and/or described in the art

Many polymer approaches have demonstrated efficacy in delivering nucleic acids in vivo into the cell cytoplasm (reviewed in de Lougerolles Hum Gene Ther. 2008, 19: 125-132). An approach using dynamic poly conjugates has been shown in vivo in mice to effectively deliver siRNA and silence endogenous target mRNA in hepatocytes. In this approach, a multicomponent polymer system includes a membrane-active polymer to which nucleic acid, is covalently coupled via a disulfide bond and where both PEG (for charge masking) and N-acetylgalactosamine (for hepatocyte targeting) groups are linked via pH-sensitive bonds. On binding to the hepatocyte and entry into the endosome, the polymer complex disassembles in the low-pH environment, with the polymer exposing its positive charge, leading to endosomal escape and cytoplasmic release of the siRNA from the polymer. Replacing the N-acetylgalactosamine group with a mannose group can alter targeting sinusoidal endothelium and Kupffer cells (Rozema et al., Proc Natl Acad Set USA. 2007, 104:12982-12887). Another approach using cyclodextrin-containing polycation nanoparticles to formulate siRNAs demonstrates targeted silencing of the EWS-FLH gene product in Ewing's sarcoma tumor cells (Hu-Lieskovan et al., Cancer Res. 2005, 65: 8984-8982); the contents of each of which are incorporated by reference in their entirety. Both of these delivery strategies incorporate rational approaches using polymers for both targeted delivery and endosomal escape mechanisms.

In some embodiments, the plurality of ASOs described herein may be formulated using naturally derived polymers, structural proteins and polysaccharides, such as cationic collagen derivatives and chitosan. Cationic collagenous proteins have been used for nucleic acid delivery to articular cartilage and bone for regenerative medicine and metastatic tumor treatment (Capito et al., Gene Ther., 2007, 14:721-732; Curtin et al., At7v. Healthc. Mater., 2015, 4:223-227). Chitosan, a linear cationic polysaccharide, is produced by the deactylation of chitin (poly-d-glucosamine), which is non-toxic even at a high concentration and can be formulated into polyplexes. A non-limiting example of chitosan-based formulation includes a core of positively charged chitosan and an outer portion of negatively charged substrate (U.S. Pub. No. US20120258176; the contents of which are herein incorporated by reference in their entirety). Chitosan includes, but is not limited to N-trimethyl chitosan, mono-N-carboxymethyl chitosan (MCC), N-palmitoyl chitosan (NPCS), EDTA-chitosan, low molecular weight chitosan, chitosan derivatives, or combinations thereof.

In some embodiments, other naturally derived carbohydrate-based polymers comprise Cyclodextrins (CDs). In some embodiments, CDs comprising a-, 0-, or y-CD forms can be used in combination with other cationic polymers for delivering nucleic acids, e.g., to the brain or other organs.

In some embodiments, the plurality of ASOs described herein may be formulated using synthetic polymers which may incorporate versatile chemistries in a controlled manner providing flexibility and more options for polynucleotide formulations. Various synthetic strategies exist in the art to control polymerization reactions and, therefore, the properties of the resulting polymer. Examples of methods include controlled free-radical polymerizations such as reversible addition-fragmentation chain transfer (RAFT) polymerization and atom transfer radical polymerization (ATRP) (Boyer et al., Chem. Rev., 2009, 109:5402-5436). The polymers formulated with the polynucleotide (e.g., ASO) compositions of the present disclosure may be synthesized by the methods known in the art.

In some embodiments, cationic groups may be incorporated to polymers for formulating nucleic acid molecules. Without wishing to be bound by any theory, cationic groups can aid with the loading of negatively charged nucleic acid cargo and facilitate the interaction with negatively charged glycoproteins on the cell membrane when delivering the loaded polynucleotides to a cell.

In some embodiments, the ASO formulations described herein may comprise at least one polymeric compound such as, but not limited to, polyethenes, polyethylene glycol (PEG), poly(l-lysine)(PLL), PEG grafted to PLL, cationic lipopolymer, biodegradable cationic lipopolymer, polyethyleneimine (PEI), cross-linked branched poly(alkylene imines), a polyamine derivative, a modified poloxamer, a biodegradable polymer, elastic biodegradable polymer, biodegradable block copolymer, biodegradable random copolymer, biodegradable polyester copolymer, biodegradable polyester block copolymer, biodegradable polyester block random copolymer, multiblock copolymers, linear biodegradable copolymer, poly[a-(4-aminobutyl)-L-glycolic acid) (PAGA), biodegradable cross-linked cationic multi-block copolymers, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, polyamines, polylysine, poly(ethylene imine), poly(serine ester), poly(L-lactide-co-L-lysine), poly(4-hydroxy-L-proline ester), acrylic polymers, amine-containing polymers, dextran polymers, dextran polymer derivatives or combinations thereof.

In some embodiments, the synthetic polymers are biodegradable. Synthetic biodegradable polymers may be generated by assembling low molecular weight monomers into polymers via bioreversible linkages such as sulfide or ester bonds. Examples of synthetic biodegradable polymers include, but are not limited to, poly(lactic acid) (PLA), poly(glycolic acid) (PGA), poly(lactic-co-glycolic acid) (PLGA), poly(beta amino) esters (PBAEs), Poly(amine-co-esters) (PACEs).

In some embodiments, biodegradable polymers have been previously used to protect nucleic acids from degradation and have been shown to result in sustained release of payloads in vivo (Rozema et al., Proc Natl Acad Sci USA. 2007, 104:12982-12887; Sullivan et al., Expert Opin Drug Deliv. 2010, 7: 1433-1446; Convertine et al., Biomacromolecules. 2010, October 1; Chu et al., Acc Chem Res. 2012, January 13; Manganiello et al., Biomaterials. 2012, 33:2301-2309; Benoit et al., Biomacromolecules. 2011, 12:2708-2714; Singha et al., Nucleic Acid Ther. 2011, 2: 133-147; de Fougerolles Hum Gene Ther. 2008, 19:125-132; Schaffert and Wagner, Gene Ther. 2008, 16:1131-1138; Chaturvedi et al., Expert Opin Drug Deliv. 2011, 8: 1455-1468; Davis, Mol Pharm. 2009, 6:659-668; Davis, Nature, 2010, 464: 1067-1070; the contents of each of which are herein incorporated by reference in their entirety). In some embodiments, the biodegradable polymers may be polymers comprising a polyethylenimine group. In some embodiments, the polymers may be a biodegradable cationic lipopolymer.

In some embodiments, the ASO formulations described herein may comprise polymeric carriers using polymers-containing different nanoparticles. For example, the therapeutic polymer nanoparticles may be identified by the methods described in US Pub No. US20120140790, the contents of which are herein incorporated by reference in their entirety. In some embodiments, the therapeutic nanoparticles may comprise a polymeric matrix. As a nonlimiting example, the nanoparticle may comprise two or more polymers and diblock copolymers such as, but not limited to, polyethylenes, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, polyamines, polylysine, poly(ethylene imine), poly(serine ester), poly(L-lactide-co-L-lysine), poly(4-hydroxy-L-proline ester) or combinations thereof.

In some embodiments, the ASO formulations described herein may comprise acrylic polymers including but not limited to, acrylic acid, methacrylic acid, acrylic acid and methacrylic acid copolymers, methyl methacrylate copolymers, ethoxyethyl methacrylates, cyanoethyl methacrylate, amino alkyl methacrylate copolymer, poly(acrylic acid), poly(methacrylic acid), polycyanoacrylates and combinations thereof. In other embodiments, the antisense polynucleotide (e.g., ASO) compositions of the present disclosure may be formulated using amine-containing polymers such as, but not limited to polylysine, polyethylene imine, poly(amidoamine) dendrimers, poly(beta-amino esters).

In some embodiments, the nanoparticles may comprise at least one degradable polyester which may contain poly cationic side chains. Degradable polyesters include, but are not limited to, poly(serine ester), poly(L-lactide-co-L-lysine), poly(4-hydroxy-L-proline ester), and combinations thereof. The degradable polyesters may include a PEG conjugation to form a PEGylated polymer. In other embodiments, the ASO compositions of the present disclosure may be formulated with at least one cross-linkable polyester. Crosslinkable polyesters include those known in the art.

In some embodiments, the plurality of ASOs are covalently attached to a carrier molecule. In an example, the ASO is covalently attached to a carbohydrate, a protein, a small molecule (e.g., a-tocopherol), a peptide (e.g., a cell-penetrating peptide), an antibody, a lipid (e.g., cholesterol) or a polymer (e.g., PEG). In an embodiment, the ASO is not covalently attached to a carrier molecule.

In some embodiments, the plurality of ASOs described herein may be conjugated to at least one targeting ligand. The targeting ligand may be any ligand known in the art such as, but not limited to, a monoclonal antibody, a peptide and a nucleic acid (e.g., aptamer). In other embodiments, the polymer nanoparticles can be selectively targeted to cells, tissues and/or organs through expression of different ligands (e.g., folate, transferrin, and N-acetylgalactosamine (GalNAc)).

In some embodiments, the polymer nanoparticles (NPs) for formulating the ASO compositions of the present disclosure may permit a sustained or delayed release of the polynucleotide compositions. The altered release profile for the antisense polynucleotide compositions can result in regulation over an extended period of time. In some embodiments, the polymeric formulations for sustained release may include, but are not limited to, PLGA microspheres, ethylene vinyl acetate (EVAc), poloxamer, and fibrinogen polymers. For example, the antisense polynucleotide compositions may be formulated in PLGA microspheres by preparing the PLGA microspheres with tunable release rates (e.g., days and weeks) and encapsulating the polynucleotide (e.g., ASO(s)) compositions in the PLGA microspheres while maintaining the integrity of the polynucleotides during the encapsulation process.

In some embodiments, the ASO formulations described herein may comprise one or more liposomes. As used herein, the term “liposome” refers to an artificially prepared vesicle which may primarily be composed of one or several lipid bilayers and may be used as a delivery vehicle. Liposomes can be of different sizes such as, but not limited to, a multilamellar vesicle (MLV) which may be hundreds of nanometers in diameter and may contain a series of concentric bilayers separated by narrow aqueous compartments, a small unicellular vesicle (SUV) which may be smaller than 50 nm in diameter, and a large unilamellar vesicle (LUV) which may be between 50 and 500 nm in diameter.

Liposome design may include, but is not limited to, opsonins or ligands in order to improve the attachment of liposomes to unhealthy tissue or to activate events such as, but not limited to, endocytosis. Liposomes may contain a low or a high pH in order to improve the delivery of the pharmaceutical formulations. The formation of liposomes may depend on the physicochemical characteristics such as, but not limited to, the pharmaceutical formulation entrapped and the liposomal ingredients, the nature of the medium in which the lipid vesicles are dispersed, the effective concentration of the entrapped substance and its potential toxicity, any additional processes involved during the application and/or delivery of the vesicles, the optimization size, polydispersity and the shelf-life of the vesicles for the intended application, and the batch-to-batch reproducibility and possibility of large-scale production of safe and efficient liposomal products. Liposomes can be cationic liposomes, neutral liposomes. Cationic liposomes have been used to deliver siRNA to various cell types (e.g., US Patent Application Publication No.: US2004/0204377).

In some embodiments, the liposome may contain a sugar-modified lipid.

In some embodiments, liposomes are formed by the self-assembly of dissolved lipid molecules and/or polymers. The ASOs of the present disclosure may be entrapped passively into the lipid bilayers through the preparation of liposomes, e.g., encapsulated in the aqueous core of the liposome or the aqueous phase between bilayers (in the case of multilamellar vesicles) using passive loading methods, such as reverse phase evaporation, dehydration-rehydration method, or active loading involving pH-gradient across the liposome membrane (Szoka and Papahadjopoulos, PNAS, 1978; 9:4194-4198; Shew and Deamer, Biochim. Biophy Acta., 1985; 1:1-8; and Mayer et al., Biochim. Biophy Acta., 1986; 1: 123-126).

The liposome formulation may be influenced by, but not limited to, the selection of the cationic lipid component, the degree of cationic lipid saturation, the nature of the PEGylation, ratio of all components and biophysical parameters such as size.

In some embodiments, liposomes may be targeted liposomes with surface-attached ligands, capable of recognizing and binding to cells of interest. The targeted liposomes may increase delivery and accumulation of liposomes and entrapped ASOs in the desired tissues and organs. The surface targeting ligands may include immunoglobulins (Ig) and their fragments, peptides and aptamers.

In some embodiments, the surface of liposomes may be coated with inert, biocompatible polymers such as PEG. The polymer coating forms a protective layer over the liposomal surface and slows down the liposome recognition by opsonins; thereby increasing circulation of liposomes in vivo.

### B. Splice-Switching ASO Activity Reporter

In some embodiments, provided herein is a method of assessing delivery and/or splice-switching activity of an antisense oligonucleotide (ASO), wherein the method utilizes an activity reporter comprising a genetic sequence that is transcribed as a pre-mRNA comprising a first segment encoding a first portion of a reporter protein, a cryptic sequence, and a second segment encoding a second portion of the reporter protein. In some embodiments, following introduction of the ASO to the transgenic mouse or cultured cells, the ASO alters splicing of the pre-mRNA to exclude the cryptic sequence from a mature mRNA transcript, thereby causing expression of the reporter protein.

In some embodiments, the cryptic sequence is a cryptic β-globin (HBB) exon. In some embodiments, the cryptic HBB exon comprises nucleotide substitutions to reduce background expression of the reporter protein. In some embodiments, the nucleotide insertions, deletions, or substitutions in the cryptic exon decrease the exclusion of the cryptic sequence in the absence of ASO by more than 10%, more than 20%, or more than 30%. In some embodiments, the nucleotide insertions, deletions, or substitutions or substitutions in the cryptic exon decrease the exclusion of the cryptic sequence in the absence of ASO by about 5% to about 50%, by about 10% to about 40%, by about 15% to about 35%, or about 10% to about 30%.

In some embodiments, the nucleotide sequence of the cryptic HBB exon is set forth in SEQ ID NO.: 1. In some embodiments, the cryptic HBB exon comprises a nucleotide sequence with at least 80, 85, 90, 95, or 100% identity to SEQ ID NO 1. In some embodiments, the cryptic HBB exon comprises a nucleotide sequence with at least 85% identity to SEQ ID NO.: 1. In some embodiments, the cryptic HBB exon comprises nucleotide substitutions at positions 657 and 658 to modify the sequence of the HBB exon from TA to GT.

In some embodiments, the nucleotide substitutions of T657G and A658T decrease the exclusion of the cryptic sequence in the absence of splice-switching ASO by more than 10% relative to the exclusion of the cryptic sequence when the nucleotide substitutions are not present. In some embodiments, the nucleotide substitutions of T657G and A658T decrease the exclusion of the cryptic sequence in the absence of splice-switching ASO by between about 10% and about 30%. In some embodiments, the nucleotide substitutions of T657G and A658T decrease the exclusion of the cryptic sequence in the absence of splice-switching ASO by at least about 10%, about 20%, or about 30%. In some embodiments, the nucleotide substitutions of T657G and A658T decrease the exclusion of the cryptic sequence in the absence of splice-switching ASO by about 26.1%. As discussed elsewhere, in some embodiments, the exclusion of the cryptic sequence from a mature mRNA transcript causes expression of a functional form of the reporter protein. While the exclusion of the cryptic sequence is supposed to be triggered upon the administration of ASO, the exclusion may occur at a base level even in the absence of ASO, rendering a base level expression of the reporter gene, i.e., “leaky expression.” This base level of cryptic sequence exclusion is significantly decreased upon the presence of the T657G and A658T substitutions, and consequently the level of leaky expression is also decreased. This decreased leaky expression allows significant improvement of sensitivity for methods monitoring the difference of signals with and without the administration of ASO. In other words, with the T657G and A658T substitutions, the method has a much lower background signal from the reporter and thus the signal that is increased after the administration of ASO becomes more apparent and easier to detect, measure and quantify. In some embodiments, the methods provided herein beneficially enables high throughput analysis of ASO features.

In some embodiments, the reporter protein comprises a detectable and/or a selectable marker. In some embodiments, the reporter protein comprises a fluorescent reporter protein. In some embodiments, the fluorescent reporter protein is selected from the group consisting of GFP, EGFP, YFP, RFP, CFP, BFP, mCherry, and near-infrared fluorescent proteins. In some embodiments, the reporter protein comprises enhanced green fluorescent protein (EGFP).

In some embodiments, the nucleotide sequence of the EGFP reporter is set forth in SEQ ID NO.: 21. In some embodiments, the EGFP reporter sequence comprises a first portion of an EGFP reporter protein, a cryptic HBB exon sequence, and a second portion of the EGFP reporter protein, wherein nucleotide substitutions at positions T1128G and A1129T modify the sequence of the HBB exon from TA to GT. In some embodiments, the EGFP reporter construct comprises a nucleotide sequence with at least 80, 85, 90, 95, or 100% identity to SEQ ID NO.: 21. In some embodiments, the EGFP reporter construct comprises a nucleotide sequence with at least 85% identity to SEQ ID NO.: 21.

In some embodiments, the reporter protein comprises a bioluminescent reporter protein. In some embodiments, the bioluminescent reporter protein comprises a class of oxidative enzymes that produce bioluminescence. In some embodiments, the bioluminescent reporter protein is selected from the group consisting of GLuc, NanoLuc (NLuc), MLuc7, HtLuc, LoLuc, PaLuc1, PaLuc2, MpLucl, McLucl, MaLucl, MoLucl, MoLuc2, MLuc39, PsLucl, LocLucl-3, HtLuc2 Renilla, TurboLucl6 (TLuc), homologs or orthologs thereof, and mutants or functional derivatives thereof. In some embodiments, the reporter protein is luciferase. In some embodiments, the reporter protein emits a signal when incubated with D-luciferin.

In some embodiments, the nucleotide sequence of the luciferase reporter is set forth in SEQ ID NO.: 22. In some embodiments, the luciferase reporter sequence comprises a first portion of a luciferase reporter protein, a cryptic HBB exon sequence, and a second portion of the luciferase reporter protein, wherein nucleotide substitutions at positions T2001G and A2002T modify the sequence of the HBB exon from TA to GT. In some embodiments, the luciferase reporter construct comprises a nucleotide sequence with at least 80, 85, 90, 95, or 100% identity to SEQ ID NO.: 22. In some embodiments, the luciferase reporter construct comprises a nucleotide sequence with at least 85% identity to SEQ ID NO.: 22.

In some embodiments, the reporter protein can be detected from a sample by fluorescence microscopy or any one or more methods for measuring light production commonly used in the art. In some embodiments, a substrate of the reporter protein is provided, which results in production of light.

In some embodiments, detected expression of the reporter protein is indicative of presence, amount, and/or splice-switching activity of the ASO in the cell.

### C. Assessing ASO Activity in Cells and Tissues

In some embodiments, provided herein is a method of assessing delivery and/or splice-switching activity of an antisense oligonucleotide (ASO), wherein the method comprises a step of introducing ASOs to a transgenic animal such as transgenic mouse. In some embodiments, provided herein is a method of assessing delivery and/or splice-switching activity of an antisense oligonucleotide (ASO), wherein the method comprises a step of introducing the ASO to a plurality of cultured cells. In some embodiments, the cultured cells are from a transgenic animal.

In some embodiments, a transgenic animal comprises any animal whose genome has been altered by the introduction of one or more foreign DNA sequences from another species or breed by artificial means. In some embodiments, the transgenic animal comprises a mammal. In some embodiments, the transgenic animal comprises a rodent, such as a mouse or rat.

In some embodiments, the ASO is provided to a plurality of cells. In some environments, the ASO is provided to a plurality of cells in a cell culture. In some embodiments, the cell culture is a three-dimensional cell culture. In some embodiments, the cell culture is a mixed cell culture. In some embodiments, the cell culture comprises multiple cell types. In some embodiments the culture comprise astrocytes, microglia, and/or cortical neurons. In some embodiments, the cells are derived from the cortex, the hippocampus, and/or the corpus callosum. In some embodiments, the ASO is provided to the plurality of cells in an LNP.

In some embodiments, the plurality of cells are incubated or cultured with the ASO for a period of time. In some embodiments, the plurality of cells are incubated or cultured with the ASO for at least 1 hour, at least 6 hours, at least 12 hours, at least 18 hours, at least 24 hours, at least 2 days, at least 3 days, at least 4 days, a least 5 days, at least 6 days, at least 7 days, at least 10 days, or at least 14 days. In some embodiments, the plurality of cells are incubated or cultured with the ASO for about 1 hour to about 14 days, about 1 hour to about 7 days, about 1 hour to about 5 days, about 1 hour to about 2 days, about 1 hour to about 1 day, about 1 day to about 14 days, about 3 days to about 14 days, or about 7 days to about 14 days. In some embodiments, a time course is performed comprising detecting the reporter protein after various incubation periods. For example, in some embodiments, the reporter protein is detected after incubation for 0, 1, 4, and 7 days. In some embodiments, the ASO is provided to the plurality of cells at a concentration of at least 0.1 μM, at least 0.2 μM, at least 0.4 μM, at least 0.5 μM, at least 1 μM, at least 2 μM, at least 3 μM, at least 4 μM, at least 5 μM, at least 10 μM, or at least 15 μM is assessed. In some embodiments, the ASO is provided to the plurality of cells at a concentration of between about 0.1 μM to about 15 μM, such as about 0.5 μM to about 10 μM, about 1 μM to about 10 μM, about 5 μM to about 10 μM, or about 1 μM to about 5 μM is assessed. In some embodiments, at least 10 μg, at least 15 μg, at least 20 μg, at least 25 μg, at least 30 μg, at least 40 μg, at least 45 μg, at least 50 μg, at least 60 μg, at least 70 μg, at least 80 μg, at least 90 μg, at least 100 μg, at least 110 μg, at least 120 μg, at least 150, or at least 175 μg of the ASO is provided to the plurality of cells. In some embodiments, an amount of between about 25 μg to about 175 μg, such as between about 25 μg and about 125 μg, about 25 μg and about 100 μg, about 25 μg and about 75 μg, about 75 μg and about 175 μg, about 50 μg and about 100 μg, or about 80 μg and about 150 μg of the ASO is provided to the plurality of cells. In some embodiments, the plurality of cells are mammalian cells. In some embodiments, the cells are incubated with ASO following cell culture. In some embodiments, prior to incubation with the ASO, the cells are fixed. In some embodiments, the cells are washed to remove the ASO. In some embodiments, the plurality of cells are imaged following fixation.

In some embodiments, the cells are transfected with a reporter cassette comprising the genetic sequence that is transcribed as the reporter protein and the cryptic sequence. In some embodiments, a cell line is established comprising the cassette.

In some embodiments, the cells are primary neurons or iPS derived neurons. In some embodiments, the plurality of cells are from a transgenic embryo. In some embodiments, the cells are mouse embryonic cortical neurons.

In some embodiments, the cells are incubated with ASO following isolation from an animal, such as a transgenic mouse. In some embodiments, tissue is dissected from the animal. In some embodiments, cells from the tissue are dissociated. In some embodiments, the cells obtained from the animal are cultured prior to incubation with the ASO. In some embodiments, multiple tissues are dissected from the animal. In some embodiments, the tissue is a neuronal tissue. In some embodiments, the tissue is liver, heart, spleen, skin, ear, tail, nose, brain, frontal or temporal cortices, lumbar, cervical, or thoracic spinal cord.

In some embodiments, the plurality of cells are human, rat, or mouse cells. In some embodiments, the plurality of cells are from a cell line. In some embodiments, the plurality of cells are primary cells.

In some embodiments, the ASO is provided directly to a transgenic animal. In some embodiments, the ASO is provided by parenteral route including intravenous, intraarterial, subcutaneous, intraperitoneal or intramuscular injection or infusion, intrathecal or intracranial, e.g. intracerebral or intraventricular, intravitreal administration. In some embodiments, the ASO is provided intravenously. In another embodiment, the ASO is provided subcutaneously. In some embodiments, the ASO is provided by intrathecal injection. In some embodiments, the ASO is provided by inhalation. In some embodiments, the ASO is provided by inhalation nasally. In some embodiments, the ASO is administered via intracerebral, intracerebroventricular or intrathecal administration. In some embodiments, the animal is a transgenic mouse.

In some embodiments, the ASO is provided directly to the transgenic animal. In some embodiments, an intravenous (IV) injection is used. In some embodiments, the ASO is administered in a bolus. In some embodiments, the ASO is administered at a dose of between about 1 μg to about 500 μg. In some embodiments, the ASO is administered at a dose of between about 1 μg to about 10 μg, about 10 μg to about 25 μg, about 25 μg to about 50 μg, about 50 μg to about 75 μg, about 75 μg to about 100 μg, about 100 μg to about 200 μg, about 200 μg to about 300 μg, about 300 μg to about 400 μg, or about 400 μg to about 500 μg. In some embodiments, the ASO is administered at a dose of about 25 μg. In some embodiments, the ASO is administered at a dose of about 100 μg. In some embodiments, the ASO is administered at a dose of about 250 μg.

In some environments, the ASO is provided directly to the central nervous system of the animal. In some embodiments, a freehand in Intracerebroventricular (ICV) injection is used. In some embodiments, the ASO is administered in a bolus. In some embodiments, the ASO is administered at a dose of between about 1 μg to about 200 μg. In some embodiments, the ASO is administered at a dose of between about 1 μg to about 10 μg, about 10 μg to about 25 μg, about 25 μg to about 50 μg, about 50 μg to about 75 μg, about 75 μg to about 100 μg, or about 100 μg to about 200 μg. In some embodiments, the ASO is administered at a dose of about 12.5 μg. In some embodiments, the ASO is administered at a dose of about 50 μg. In some embodiments, the ASO is administered at a dose of about 100 μg. In some embodiments, the ASO is administered at a rate of 0.1 μl/second to 10 μl/second, such as about 0.5 μl/second, about 1 μl/second, about 2 μl/second, or about 5 μl/second. In some embodiments, the animal is a transgenic mammal. In some embodiments, the animal is a transgenic mouse.

In some embodiments, the ASO is provided to the cerebrospinal fluid (CSF) of the animal. In some embodiments, the ASO is provided by direct injection into the CSF. In some embodiments the direct injection into the CSF is a bolus. In some embodiments, the ASO is provided to the CSF by injection in the spinal column or between vertebrae. In some embodiments, the ASO is provided to the CSF by injection into the fluid around the brain. In some embodiments, the ASO is administered at a rate of 0.1 μl/second to 10 μl/second, such as about 0.5 μl/second, about 1 μl/second, about 2 μl/second, or about 5 μl/second. In some embodiments, the animal is a transgenic mammal. In some embodiments, the animal is a transgenic mouse.

In some embodiments, the ASO is provided to the animal by injection, such as intrathecal injection. In some embodiments, the ASO is provided to the animal by direct injection into the spinal canal or into the subarachnoid space. In some embodiments, the ASO is administered in a bolus. In some embodiments, the ASO is administered at a rate of 0.1 μl/second to 10 μl/second, such as about 0.5 μl/second, about 1 μl/second, about 2 μl/second, or about 5 μl/second.

In some environments, the ASO is provided directly to the eye of the animal. In some embodiments, an intravitreal (IVT) injection is used. In some embodiments, the ASO is administered in a bolus. In some embodiments, the ASO is administered at a dose of between about 1 μg/eye to about 100 μg/eye. In some embodiments, the ASO is administered at a dose of between about 1 μg/eye to about 10 μg/eye, about 10 μg/eye to about 25 μg/eye, about 25 μg/eye to about 50 μg/eye, about 50 μg/eye to about 75 μg/eye, or about 75 μg/eye to about 100 μg/eye. In some embodiments, the ASO is administered at a dose of about 6.25 μg/eye. In some embodiments, the ASO is administered at a dose of about 25 μg/eye. In some embodiments, the ASO is administered at a dose of about 50 μg/eye. In some embodiments, the ASO is administered at a rate of 0.1 μl/second to 10 μl/second, such as about 0.5 μl/second, about 1 μl/second, about 2 μl/second, or about 5 μl/second. In some embodiments, the animal is a transgenic mammal. In some embodiments, the animal is a transgenic mouse.

In some embodiments, the reporter protein is detected in multiple tissues and/or organs in a living or deceased transgenic animal. In some embodiments, the reporter protein is detected in any of liver, brain, spleen, heart, skin ear, lung, muscle, CNS, kidney, pancreas, colon, testes, prostate, gallbladder, thyroid, esophagus, gland, uterus, stomach, intestines, spinal cord.

### D. Transgene Structure

In some embodiments, provided herein is a method of assessing delivery and/or splice-switching activity of an antisense oligonucleotide (ASO) in a plurality of cells which comprise a genetic sequence transcribing a pre-mRNA which comprises a first segment encoding a first portion of a reporter protein, a cryptic sequence, and a second segment encoding a second portion of the reporter protein.

In some embodiments, the genetic sequence transcribing a pre-mRNA is designed to express the activity reporter in cultured cell lines. In some embodiments, the cultured cells stably express a nuclear localized CRE recombinase. In some embodiments, the activity reporter construct comprises, from N-terminus to C-terminus, a piggyBac inverted terminal repeat, a promoter, a loxp-stop-loxp cassette, mKate2, a self-cleaving peptide (e.g. P2A), the splice-switching ASO activity reporter, and a piggyBac inverted terminal repeat. In some embodiments, the reporter protein vector is cloned into a pBR322 vector. In some embodiments, stable cell lines are obtained following integration of the genetic sequence via piggyBac transposase activity. In some embodiments, FACS is used to isolate mKate2 positive cells to obtain a stable cell line.

In some embodiments, the cell line is an immortalized mammalian cell line. In some embodiments, the cell line is derived from a fibroblast cell. In some embodiments, the cell line is derived from an epithelial cell. In some embodiments, the cell line is derived from an endothelial cell. In some embodiments, the cell line is derived from monocyte or macrophage. In some embodiments, the cell line is derived from a lymphocyte. In some embodiments, the cell line is derived from a myoblast cell. In some embodiments, the cell line is derived from a neuronal cell. In some embodiments, the cell line is adherent. In some embodiments, the cell line is semi-adherent. In some embodiments, the cell line is a suspension cell line. In some embodiments, the cell line is a mouse cell line. In some embodiments, the cell line is a human cell line.

In some embodiments, the cell line is selected from the group consisting of 293, 3T6, A549, A9, BALB/3T3, BHK-21, BHL-100, BT, Caco-2, Chang, CHO-K1, Clone 9, Clone M-3, COS-1, COS-3, COS-7, CRFK, CV-1, D-17, GH1, GH3, HCT-15, HeLa, HT-1080, HT-29, HUVEC, I-10, JEG-3, Jensen, L2, LLC-WRC 256, McCoy, MCF7, WI-38, WISH, XC, Y-1, HCT116, PANC1, NIH3T3, A431, 3T3L1, U20S, MDAMB231, NTERA2, RAW 264.7, T47D, PC3, C2C12, NCCIT, SKNAS, L6, GTL16, B16F10, C8D1A, HAEC, F9, HSkMs, SKOV3, LNCAP, A375, H1975, SW480, SKBR3, RD, SCC15, HMEC-1, CAKI-1, RSC96, OVCAR3, NMUMG, AML12, SKMEL-31, MEF-IM, BEWO, 769P, MCF10A, LOVO, H1299, A673, BT-549, H4, HACAT, Htert-RPE1, HEK293, RT4, U118MG, SW620, 786-O, MJ, Colo205, MIAPACA2, KATO-III, AtT-20, Daudi, H9, HL-60, IM-9, Jurkat, K-562, KG-1, AML193, U937, THP1, PC12, RAJI, NK92, P388D1, RAMOS, EB2, SNU5, MOLT4, JEKO1, KARPAS 299, HEL 92.1.7, 8A3B.6, REH, EL4, M1, JM1, MV411, ARH77, KASUMI-1, TF1, Expi293, and ExpiCHO. In some embodiments, the cell line is HEK293. In some embodiments, the cell line stably expresses a nuclear localized CRE recombinase.

In some embodiments, the genetic sequence transcribing a pre-mRNA is designed to express the activity reporter in a mammalian organism. In some embodiments, the mammalian organism comprises a rodent such as a mouse or rat. In some embodiments, the mammalian organism comprises a mouse. In some embodiments, the activity reporter construct comprises, from N-terminus to C-terminus, a left homology arm, a promoter, a loxp-stop-loxp cassette, mKate2, a self-cleaving peptide (e.g. P2A), the splice-switching ASO activity reporter, and a right homology arm.

In some embodiments, the homology arm is complementary to a safe harbor locus in the mouse genome. In some embodiments, a safe harbor locus is defined as a locus in the genome of a host cell which is not affected in any adverse manner by the host cell after knocking in an exogenous gene, and at the same time, expression of the exogenous gene is not affected by the surrounding region. Non-limiting examples of safe harbor loci in mammalian cells are the Rosa26 locus, the Hipp11 locus. In some embodiments, the Rosa26 locus is a locus for exogenous genome formation and ubiquitous expression. In some embodiments, the safe harbor locus is the Rosa26 locus.

In some embodiments, a transgenic mouse is generated via homologous recombination between the homology arms in the transgenic reporter and the native safe harbor loci in a mouse cell. In some embodiments, the CRISPR/Cas9 system is employed to improve knock-in efficiency of the transgenic reporter. In some embodiments, a ROSA26 guide sequence (SEQ ID NO:2) targets the CRISPR/Cas9 system to generate a DNA break at the ROSA26 locus and improve knock-in efficiency of the transgenic reporter. In some embodiments, the transgenic reporter and CRISPR/Cas9 system is microinjected into both pronuclei of E1.5 two-cell-stage C57BL/6J embryos. In some embodiments, the microinjected embryos are then transferred into the oviducts of pseudo pregnant female mice.

In some embodiments, G0 mosaic founder mice are analyzed to confirm integration of the transgenic reporter at the ROSA26 locus. In some embodiments, loss-of-allele (LOA) assays and full-length sequence verification is conducted on G0 mosaic founder mice. In some embodiments, the LOA assays utilize primers to detect the transgenic reporter sequence (SEQ ID NOs: 3-8). In some embodiments, LOA-positive animals are then verified by sequencing the full-length insert. In some embodiments primers to detect the full-length insert were designed (SEQ ID NOs: 9-16). In some embodiments, nested-long range PCR is utilized to generate a template for amplicon sequencing. In some embodiments, sequence verified G0 animals at breeding age (>6 weeks) were crossed with C57BL/6J animals to produce G1 animals. In some embodiments, G1 animals were pre-screened using LOA assays to identify targeting ki animals have 1 copy of the native Rosa26 insertion site and 1 copy of the ki donor. In some embodiments, single ROSA26 insertion animals were then transferred into a production colony.

In some embodiments, the DNA cassette comprises a promoter to drive expression of the splice-switching ASO activity reporter. In some embodiments, the promoter is a foreign promoter. In some embodiments, the promoter is functional in a mammalian cell and can express the splice-switching ASO activity reporter at a desired level. In some embodiments, the promoter is operably linked to the nucleotide sequence of the splice-switching ASO activity reporter. In some embodiments, the promoter is operably linked to drive expression of mKate2 and a splice switching ASO activity reporter comprising ssEGFP.HBB. In some embodiments, the promoter is operably linked to drive expression of mKate2 and a splice switching ASO activity reporter comprising ssLuc2.HBB.

In some embodiments, the promoter is a constitutive promoter. In some embodiments, a constitutive promoter is an unregulated promoter that continuously drives transcription of the desired DNA cassette. In some embodiments, the constitutive promoter does not need specific stimuli to drive expression of the desired DNA cassette. In some embodiments the constitutive promoter is selected from the group consisting of CMV, EF1a, SV40, PGK1, Ubc, human beta actin, CAG, UAS, Ac5, Polyhedrin, TEF1, GDS, CaMV355, Ubi, H1, and U6.

In some embodiments, the promoter is an inducible promoter. In some embodiments, an inducible promoter initiates transcription only after receiving a stimulus. In some embodiments, an inducible promoter can be stimulated by chemical agents, steroids, alcohol, temperature, mechanical injury, oxygen, or light. In some embodiments, the inducible promoter is selected from the group consisting of TRE, Gal1, Gal10, and ADH1.

In some embodiments, the DNA cassette comprise a promoter that causes transcription in multiple cell types and/or tissue types. In some embodiments, the promoter causes ubiquitous transcription in a transgenic animal. In some embodiments, the promoter is a strong promoter. In some embodiments, the promoter is selected from the group consisting of SV40, CMV, UBC, EF1A, PGK, B-actin, CAG, and CAGG. In some embodiments, the promoter is a mammalian promoter, such as a murine promoter.

In some embodiments, the DNA cassette comprises a promoter that causes transcription only in one or more types of cells, e.g., neuronal cells. In some embodiments, the promoter causes transcription in CNS cells. In some embodiments, the promoter causes transcriptions in neurons, microglia, and astrocytes. In some embodiments, the DNA cassette comprises the GAD67, homeobox Dlx5/6 GLuR1, E/SYN, GFAP, CMV, NSE, synapsin, CaMKII, SynI-minCMV Syn1, or CBh promoter. In some embodiments, the promoter is a mammalian promoter, such as a murine promoter.

In some embodiments, the promoter is a cytomegalovirus (CMV) promoter. In some embodiments, the CMV promoter is a minimal CMV promoter. In some embodiments, the promoter is a CMV early enhancer promoter. In some embodiments, the promoter is a B-actin promoter. In some embodiments, the promoter is a CMV early enhancer/chickenβ-actin (CAG) promoter.

In some embodiments, tissue specific expression of the splice-switching ASO activity reporter is regulated by a loxP-stop-loxP (LSL) cassette upstream of the mKate2 fluorescent protein sequence and the splice-switching ASO activity reporter sequence. In some embodiments, the LSL cassette comprises a LoxP-flanked (aka floxed) triple repeat of the SV40 polyadenylation sequence. In some embodiments, Cre recombinase activity will permanently remove the 3×SV40 polyadenylation sequence from the reporter transgene, thereby allowing transcription of mKate2 and the splice-switching ASO activity reporter. In some embodiments the LSL cassette enables Cre-mediated conditional activation of reporter expression in mammalian cells and animals.

In some embodiments, provided herein is a method comprising the delivery of a plurality of ASOs to a transgenic mouse or cells in culture isolated from the transgenic mouse, wherein the transgenic mouse also expresses a Cre recombinase. In some embodiments, provided herein is a method comprising the delivery of a plurality of ASOs to a plurality of cultured cells, wherein the cultured cells also expresses a Cre recombinase. In some embodiments, Cre catalyzes the rearrangement of DNA sequences that contain loxP sites. In some embodiments, recombination between two loxP sites (catalyzed by the cre recombinase) causes, in certain cases, the loss of sequences flanked by these sites.

In some embodiments, Cre recombinase expression is driven by a constitutive promoter. In some embodiments, the Cre recombinase is expressed in all cells of the transgenic mouse. In some embodiments, the Cre recombinase expressed constitutively in the cultured cell line. In some embodiments, a transgenic mouse or cultured cell expressing Cre recombinase from the CAG promoter expresses Cre ubiquitously. In some embodiments, a transgenic mouse or cultured cell expressing Cre recombinase from the EF1a promoter expresses Cre ubiquitously.

In some embodiments, Cre recombinase expression is driven by a native promoter. In some embodiments, the native promoter restricts Cre expression to specific cell types. In some embodiments, the native promoter restricts Cre expression spatiotemporally. In some embodiments, the native promoter is a mammalian promoter. In some embodiments, the native promoter is a mouse promoter.

In some embodiments, the promoter that drives Cre expression is specific to a certain type of cells such as neuronal cells. In some embodiments, a transgenic mouse expressing Cre recombinase from the GFAP promoter expresses Cre in Astrocytes and/or neuronal stem cells. In some embodiments, a transgenic mouse expressing Cre recombinase from the Nestin promoter expresses Cre in neuronal stem cells or precursor cells. In some embodiments, a transgenic mouse expressing Cre recombinase from the CAG promoter expresses Cre in neuronal stem cells or precursor cells. In some embodiments, a transgenic mouse expressing Cre recombinase from the Synapsin I promoter expresses Cre in all neurons. In some embodiments, the neuronal specific Cre promoter is selected from the group consisting of Agrp, GFAP, Mnx1, Nkx2-1, Omp, Pomc1, Pvalb, Slc17a6, Sst, Syn1, Thy1, and Vip.

In some embodiments, the promoter that drives Cre expression is specific to one or more cell types present in a specific organ. In some embodiments the Cre promoter drives Cre expression in the lung, heart, liver, stomach, gallbladder, spleen, pancreas, kidneys, large intestine, small intestine, ureter, bladder, rectum, ovaries, or testes. In some embodiments, mating a transgenic mouse comprising the splice-switching ASO activity reporter transgene with a transgenic mouse comprising a cell-type or tissue specific Cre recombinase may result in progeny comprising both the splice-switching ASO activity reporter transgene and the cell-type or tissue specific Cre recombinase. In some embodiments, a transgenic mouse expressing both the splice-switching ASO activity reporter transgene and the cell-type or tissue specific Cre recombinase will result in a functional splice-switching ASO activity reporter only in the cells expressing the Cre recombinase.

In some embodiments, the cassette comprises from 5′ to 3′ a promoter, a first segment of a genetic sequence encoding a first portion of a first reporter protein, a cryptic sequence, and a second segment of a genetic sequence encoding a second portion of the first reporter. In some embodiments, the cassette comprises a genetic sequence encoding a second reporter protein. In some embodiments, the second reporter protein is 5′ or 3′ of the genetic sequences encoding the first reporter protein. In some embodiments, the first and second reporter proteins are operably linked to the same promoter. In some embodiments, the cassette comprises from 5′ to 3′ a promoter, a genetic sequence encoding a second reporter protein, a nucleic acid encoding a ribosomal skip sequence, a first segment of a genetic sequence encoding a first portion of a first reporter protein, a cryptic sequence, and a second segment of a genetic sequence encoding a second portion of the first reporter protein. In some embodiments, the first reporter protein is EGFP or luciferase. In some embodiments, the second reporter protein is mKate. In some embodiments, the cassette further comprises a loxp-stop-loxp sequence upstream of the genomic sequence encoding the first and/or second reporter protein. In some embodiments, crossing an animal comprising the cassette comprising the loxp-stop-loxp sequence with an animal expressing Cre causes expression of the first and/or second reporter protein. In some embodiments, the animal expressing Cre expresses Cre specifically in one or more tissues, such as neuronal tissues. In some embodiments, crossing the animal with the cassette with an animal with a tissue-specific Cre results in expression of the first reporter protein or first and second reporter proteins in a subset of cells.

In some embodiments, expression of a functional first reporter protein provides a quantitative readout for ASO activity. In some embodiments, the second reporter is constitutively expressed. In some embodiments the expression of the second reporter is not affected by the presence of an ASO. In some embodiments, expression of the second reporter provides a readout for the presence or incorporation of the splice-switching ASO activity reporter cassette.

### E. Methods of Detection

In some embodiments, provided herein is a method of assessing a feature of an antisense oligonucleotide in a cell comprising providing an ASO to the cell, wherein the ASO regulates splicing of a cryptic exon located in a reporter gene such that the ASO alters splicing of the mRNA encoding the reporter and expression of a functional reporter. In some embodiments, the reporter protein is detected to assess the activity of the ASO. In some embodiments, the level of the reporter protein is proportional or is correlated with the activity of the ASO. In some embodiments, the level of the reporter protein is proportional or correlated with the ability of the ASO to modulate splicing. In some embodiments, the expression of the reporter protein is measured by detecting luminescence or fluorescence. In some embodiments, expression of the reporter is detected in cells. In some embodiments, expression of the reporter protein is detected by a plate reader. In some embodiments, the reporter protein is detected using optical imaging. In some embodiments, the reporter protein is detected using cell sorting, such as FACS. In some embodiments, expression of the reporter protein is detected in live cells. In some embodiments, the cells are obtained from an animal. In some embodiments, the cells are obtained from cell culture. In some embodiments, the reporter protein is detected in fixed cells. In some embodiments, the reporter protein is a luminescent or fluorescent protein. In some embodiments, luminescence or fluorescence is detected using a microscope. In some embodiments, luminescence or fluorescence is detected using images captured by a combination of photomultiplier tubes and hybrid detectors. In some embodiments, the reporter protein is detected by detecting fluorescent or luminescent intensity. In some embodiments, the fluorescent or luminescent intensity is normalized to the number of cells.

In embodiments, expression of the reporter protein is detected in a mammal, such as a rat or a mouse. In some embodiments, expression of the fluorescent or luminescent reporter protein is detected in a living mammal. In some embodiments, expression of the fluorescent or luminescent reporter protein is detected in a deceased mammal. In some embodiments the mammal is anesthetized prior to detection. In some embodiments an optical imaging system is used to detect fluorescence or luminescence.

In some embodiments, the reporter protein is detected using immunofluorescence staining. In some embodiments, the reporter protein is detected using an antibody that binds to the reporter protein. In some embodiments, the antibody is detectably labeled. In some embodiments, the antibody is detected using a secondary antibody that is detectably labeled. In some embodiments, the amount of the reporter protein is normalized to the amount of nucleic acid in the cell. In some embodiments, the amount of the reporter protein is normalized to DAPI staining of the cells. In some embodiments, a top hat transformation is used to remove background. In some embodiments, a threshold is used to identify positively stained pixels.

In some embodiments, the methods provided herein further comprise detecting one or more ASOs in a cell or in an animal. In some embodiments, the ASO is detected using immunofluorescence. In some embodiments, the ASO is detected using an antibody that binds to the ASO. In some embodiments, the ASO is detected using an antibody that binds to a tag that is linked to the ASO. In some embodiments, the antibody is detectably labeled. In some embodiments, the antibody is detected using a secondary antibody that is detectably labeled.

In some embodiments the methods provided herein comprise imaging multiple tissues or multiple tissue sections obtained from an animal, such as a rat or a mouse. In some embodiments, the multiple tissue sections are from any of liver, brain, spleen, heart, skin ear, lung, muscle, CNS, kidney, pancreas, colon, testes, prostate, gallbladder, thyroid, esophagus, gland, uterus, stomach, intestines, spinal cord. In some embodiments, the tissues are imaged in a living animal.

In some embodiments, both an ASO and the reporter protein are detected. In some embodiments, one or more additional proteins are detected. In some embodiments, one or more additional proteins that indicate a particular cell type are detected. In some embodiments, one or more neuronal proteins are detected. In some embodiments, relative amounts, or ratios of the ASO, the reporter protein, and one or more additional cell-type specific proteins are detected. In some embodiments, any of Olig2, S100B, NeuN, Iba1 are detected. In some embodiments, the detection is in a tissue section or a live animal.

In some embodiments the method further comprises detecting a second reporter protein that does not have a cryptic exon. In some embodiments, the second reporter protein is used to identify cells that contain a cassette comprising the genetic sequence encoding the pre-mRNA comprising the reporter protein and the cryptic exon. In some embodiments, the second reporter protein is used to detect successful transformation with a vector comprising the cassette. In some embodiments, the second reporter protein is different from the first reporter protein. For example, in some embodiments, the cassette comprises a pre-mRNA encoding luciferase or EGFP and a cryptic exon that regulates splicing of the luciferase or the eGFP and a second reporter protein that is neither luciferase or EGFP. In some embodiments, the second reporter protein is a fluorescent protein. In some embodiments, the second reporter protein is mKate. In some embodiments, the ratio of the second reporter protein to the first reporter protein, the ASO, and/or a cell type specific protein is determined.

In some embodiments, provided herein are high throughput methods of determining a feature of an ASO. Beneficially such methods allow screening of a multitude of ASOs in a short amount of time, which may aid in optimization of various parameters related to formulation, delivery, pharmacokinetics, pharmacodynamics biodistribution, cellular location, update, tissue localization and the like, as provided herein. In some embodiments, the method comprises culturing a plurality of cells that comprise a genetic sequence encoding a pre-mRNA comprising a first segment encoding a first portion of a reporter protein, a cryptic sequence, and a second segment encoding a second portion of the reporter protein with an ASO. In some embodiments, the cells are cultured in a multi-well plate format. In some embodiments, the cells are cultured in a 6, 12, 24, 48, 96 or 384 well plate format. In some embodiments, the cell culture process is automated. In some embodiments, detection of the reporter protein is automated. In some embodiments, the reporter protein is detected using a plate reader. In some embodiments, fluorescence quantitation is automated. In some embodiments, image analysis is automated.

In some embodiments, the methods provided herein comprise detection of a reporter protein over a period of time. In some embodiments, the reporter protein is detected multiple times in the same sample, or same animal, over a period of one or more hours or one or more days. In some embodiments, detecting the reporter protein multiple times allows determination of the pharmacokinetics or pharmacodynamics of the ASO. In some embodiments, detecting the reporter protein multiple times provides an assessment of the clearance rate of the ASO. In some embodiments, monitoring the reporter protein over a period of time is used to determine the biodistribution of the ASO over a period of time. In some embodiments, detecting the reporter protein multiple times can be used to assess biodistribution.

In some embodiments, the methods provided herein are used to assess a feature of an ASO. In some embodiments, a feature is a formulation, a clearance rate, a delivery route, pharmacokinetics, pharmacodynamics, uptake, tissue localization, concentration, cellular internalization, cellular tracking, or biodistribution of the oligo.

In some embodiments, the methods provided herein are used to assess a formulation of an ASO. In some embodiments, multiple different formulations are assessed. In some embodiments, different formulations of an ASO with the same sequence are assessed. In some embodiments, the formulation is a pharmaceutical formulation. In some embodiments, the formulation is sterile. In some embodiments, the effects of various buffers, salts, pH, excipients, or lipids are assessed. In some embodiments, the effect of lipids, lipidoids, liposomes, lipid nanoparticles, polymers, lipoplexes, core-shell nanoparticles, peptides, proteins, nucleic acid molecules, cells, organelles, or explants is assessed. In some embodiments, the ratio of various components of the formulation is assessed. In some embodiments, cationic lipids are assessed. In some embodiments, a polymer used to deliver the ASO is assessed. In some embodiments, polyethenes, polyethylene glycol (PEG), poly(l-lysine)(PLL), PEG grafted to PLL, cationic lipopolymer, biodegradable cationic lipopolymer, polyethyleneimine (PEI), cross-linked branched poly(alkylene imines), a polyamine derivative, a modified poloxamer, a biodegradable polymer, elastic biodegradable polymer, biodegradable block copolymer, biodegradable random copolymer, biodegradable polyester copolymer, biodegradable polyester block copolymer, biodegradable polyester block random copolymer, multiblock copolymers, linear biodegradable copolymer, poly[a-(4-aminobutyl)-L-glycolic acid) (PAGA), biodegradable cross-linked cationic multi-block copolymers, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, polyamines, polylysine, poly(ethylene imine), poly(serine ester), poly(L-lactide-co-L-lysine), poly(4-hydroxy-L-proline ester), acrylic polymers, amine-containing polymers, dextran polymers, dextran polymer derivatives or combinations thereof is assessed. In some embodiments, a synthetic polymer is assessed. In some embodiments, and acrylic polymer is assessed. In some embodiment, naturally derived polymers, structural proteins and polysaccharides, such as cationic collagen derivatives and/or chitosan are assessed. In some embodiments, the effect of the formulation of ASO activity is assessed. In some embodiments, the effect of the formulation on cell penetration, release, or biodistribution is assessed. In some embodiments, the method comprises identifying or selecting a formation that provides a desired pharmacokinetic or pharmacodynamic profile. In some embodiments, the method comprises identifying or selecting a formation that provides a desired uptake. In some embodiments, the method comprises identifying or selecting a formation that provides a desired tissue localization. In some embodiments, the method comprises identifying or selecting a formation that provides a desired tissue cell penetration. In some embodiments, the pharmacokinetic property is assessed by providing the ASO to a mammal and detecting the reporter protein in the mammal. In some embodiments, the formulation is assessed by proving the ASO to a plurality of cells in cell culture.

In some embodiments, the clearance rate of the ASO is assessed. In some embodiments, the clearance rate of the ASO is assessed by detecting the reporter protein and/or the presence or absence of the ASO. In some embodiments, the clearance rate is determined by detecting the reporter protein or the ASO more than once over a period of time, for example, every hour. In some embodiments, the effect of clearance rate on ASO activity is assessed. In some embodiments, the effect of a formulation, a concentration, or a delivery route on the clearance rate is assessed.

In some embodiments, one or more delivery routes for the ASO is assessed. In some embodiments, the method comprises comparing one or more delivery routes for an ASO. In some embodiments, the delivery route is selected from the group consisting of nasal, inhalation, injection, intrathecal injection, parenteral, intravenous, intramuscular, subcutaneous, oral, rectal, ophthalmic, topical. In some embodiments, the delivery route is direct injection into the central nervous system or the cerebrospinal fluid. In some embodiments, the effect of a delivery route on ASO activity is assessed. In some embodiments, the method comprises identifying or selecting a delivery route that provides a desired pharmacokinetic or pharmacodynamic profile. In some embodiments, the method comprises identifying or selecting a delivery route that provides a desired uptake. In some embodiments, the method comprises identifying or selecting a delivery route that provides a desired tissue localization. In some embodiments, the method comprises identifying or selecting a delivery route that provides a desired tissue cell penetration. In some embodiments, the pharmacokinetic property is assessed by providing the ASO to a mammal and detecting the reporter protein in the mammal.

In some embodiments, pharmacokinetics of an ASO is assessed. In some embodiments, the pharmacokinetics of the ASO is assessed by detecting the reporter protein and/or the presence or absence of the ASO. In some embodiments, the pharmacokinetics is determined by detecting the reporter protein or the ASO more than once over a period of time, for example, every hour. In some embodiments, the relationship between pharmacokinetics and ASO activity is assessed. In some embodiments, the effect of a formulation, a concentration, or a delivery route on the pharmacokinetics is assessed. In some embodiments, the method comprises identifying or selecting a delivery route that provides a desired pharmacokinetic profile. In some embodiments, the method comprises identifying or selecting a formulation that provides a desired pharmacokinetic property. In some embodiments, the method comprises identifying or selecting a concentration that provides a desired pharmacokinetic property. In some embodiments, the pharmacokinetic property is assessed by providing the ASO to a mammal and detecting the reporter protein in the mammal.

In some embodiments, pharmacodynamics of an ASO is assessed. In some embodiments, the pharmacodynamics of the ASO is assessed by detecting the reporter protein and/or the presence or absence of the ASO. In some embodiments, the reporter protein and/or the ASO is detected in a time course. In some embodiments, the pharmacodynamics is determined by detecting the reporter protein or the ASO more than once over a period of time, for example, every hour. In some embodiments, the relationship between pharmacodynamics and ASO activity is assessed. In some embodiments, the effect of a formulation, a concentration, or a delivery route on the pharmacodynamics is assessed. In some embodiments, the method comprises identifying or selecting a delivery route that provides a desired pharmacodynamic profile. In some embodiments, the method comprises identifying or selecting a formulation that provides a desired pharmacodynamic property. In some embodiments, the method comprises identifying or selecting a concentration that provides a desired pharmacodynamics property. In some embodiments, the pharmacodynamic property is assessed by providing the ASO to a mammal and detecting the reporter protein in the mammal.

In some embodiments, uptake of an ASO is assessed. In some embodiments, cellular uptake is assessed. In some embodiments, cellular internalization is assessed. In some embodiments, the uptake of the ASO is assessed by detecting the reporter protein and/or the presence or absence of the ASO. In some embodiments, the uptake is determined by detecting the reporter protein or the ASO more than once over a period of time, for example, every hour. In some embodiments, the relationship between uptake and ASO activity is assessed. In some embodiments, the effect of a formulation, a concentration, or a delivery route on uptake is assessed. In some embodiments, the method comprises identifying or selecting a delivery route that provides a desired uptake rate, uptake amount, or ratio of the ASO. In some embodiments, the method comprises identifying or selecting a formulation that provides a desired uptake rate, uptake amount, or ratio of the ASO. In some embodiments, the method comprises identifying or selecting a concentration that provides a desired uptake rate, uptake amount, or ratio of the ASO. In some embodiments, uptake is assessed in a plurality of cells in cell culture. In some embodiments, uptake is assessed in a mammal. In some embodiments, uptake is assessed in a plurality of cells in cell culture in a high throughput manner.

In some embodiments, tissue localization of an ASO is assessed. In some embodiments, the tissue localization of the ASO is assessed by detecting the reporter protein and/or the presence or absence of the ASO in multiple tissues. For example, in some embodiments, the reporter protein is detected in two or more, three or more, four or more, five or more or ten or more tissues. By way of example, in some embodiments, the reporter protein is detected in neuronal, epithelial, connective, or muscle tissue. In some embodiments, the reporter protein is detected in one or more organs. In some embodiments, the reporter protein is detected in the brain or spine. In some embodiments, the ability of an ASO to cross the blood-brain barrier is assessed. In some embodiments, the tissue localization is assessed by detecting the reporter in a mammal. In some embodiments, the reporter protein and/or the ASO is detected in a time course. In some embodiments, the effect of concentration or formulation on tissue localization of the ASO is assessed. In some embodiments, a formulation or concentration is identified or selected that has a desired tissue localization. In some embodiments, tissue localization is assessed in a mammal.

In some embodiments, the concentration of an ASO is assessed. In some embodiments, the concentration of the ASO is assessed by detecting the reporter protein and/or the presence or absence of the ASO. In some embodiments, multiple concentrations of the same ASO are assessed. In some embodiments, the method comprises assessing the effect of ASO concentration by detecting the reporter protein or the ASO more than once over a period of time, for example, every hour. In some embodiments, the relationship between concentration and ASO activity is assessed. In some embodiments, the effect of different concentrations, pharmacokinetics, pharmacodynamics, uptake, tissue distribution, biodistribution, and/or cellular internalization is assessed. In some embodiments, the method comprises identifying or selecting a concentration that provides a desired pharmacokinetic profile. In some embodiments, the method comprises identifying or selecting a concentration that provides a desired pharmacodynamic property. In some embodiments, the concentration is assessed by providing the ASO to a mammal and detecting the reporter protein in the mammal. In some embodiments, the concentration is assessed by providing the ASO to a plurality of cultured cells and detecting the reporter protein. In some embodiments, a concentration of at least 0.1 μM, at least 0.2 μM, at least 0.4 μM, at least 0.5 μM, at least 1 μM, at least 2 μM, at least 3 μM, at least 4 μM, at least 5 μM, at least 10 μM, at least 15 μM, at least 20 μM, at least 25 μM, or at least 30 μM is assessed. In some embodiments, a concentration of between about 0.1 μM to about 30 μM, such as about 0.5 μM to about 10 μM, about 1 μM to about 10 μM, about 5 μM to about 10 μM, or about 1 μM to about 5 μM is assessed. In some embodiments, an amount of at least 10 μg, at least 15 μg, at least 20 μg, at least 25 μg, at least 30 μg, at least 40 μg, at least 45 μg, at least 50 μg, at least 60 μg, at least 70 μg, at least 80 μg, at least 90 μg, at least 100 μg, at least 110 μg, at least 120 μg, at least 150, or at least 175 μg is assessed. In some embodiments, an amount of between about 25 μg to about 175 μg, such as between about 25 μg and about 125 μg, about 25 μg and about 100 μg, about 25 μg and about 75 μg, about 75 μg and about 175 μg, about 50 μg and about 100 μg, or about 80 μg and about 150 μg and any amount or concentration within the foregoing ranges is assessed.

In some embodiments, cellular internalization of the ASO is assessed. In some embodiments, the effect of ASO concentration or formulation on cellular internalization is assessed. In some embodiments, the method comprises assessing the effect of ASO cellular internalization by detecting the reporter protein or the ASO more than once over a period of time, for example, every hour. In some embodiments, the relationship between cellular internalization and ASO activity is assessed. In some embodiments, a concentration or formulation is identified or selected to provide a desired cellular internalization. In some embodiments, cellular internalization is assessed by detecting the reporter protein in a plurality of cells in cell culture. In some embodiments, cellular internalization is assessed in a plurality of cells in cell culture. In some embodiments, cellular internalization is assessed in a mammal. In some embodiments, cellular internalization is assessed in a plurality of cells in cell culture in a high throughput manner.

In some embodiments, biodistribution of the ASO is assessed. In some embodiments, the activity of an ASO is compared across organs and tissues within a transgenic animal. In some embodiments, ASO activity is quantified in any tissue that expresses the splice-switching ASO activity reporter. In some embodiments, the effect of ASO concentration or formulation on biodistribution is assessed. In some embodiments, the biodistribution of the ASO is assessed by detecting the reporter protein and/or the presence or absence of the ASO in multiple tissues. For example, in some embodiments, the reporter protein is detected in two or more, three or more, four or more, five or more or ten or more tissues. By way of example, in some embodiments, the reporter protein is detected in neuronal, epithelial, connective, or muscle tissue. In some embodiments, the reporter protein is detected in one or more organs. In some embodiments, the reporter protein is detected in the brain or spine. In some embodiments, the ability of an ASO to cross the blood-brain barrier is assessed. In some embodiments, the biodistribution is assessed by detecting the reporter in a mammal. In some embodiments biodistribution to neuronal tissue or the liver is assessed. In some embodiments, the reporter protein and/or the ASO is detected in a time course. In some embodiments, a formulation or concentration is identified or selected that has a desired biodistribution, such as localization in neurons or neuronal tissues. In some embodiments, tissue localization is assessed in a mammal.

In some embodiments one or more modifications to the ASO is assessed. In some embodiments, multiple ASOs comprising the same sequence and different modifications are assessed. In some embodiments, the modification comprises a modification to the phosphate backbone and/or a modification to the sugar. In some embodiments, the modification is selected from the group consisting of MOE, 2′-OMe, LNA, GalNAc, 5′ methylcytosine, a phosphorothioate bond, a phosphorodithioate bond, an alkylphosphonate bond, a phosphoroamidate bond, a boranophosphate bond, and/or a morpholino ring. In some embodiments, the effect of one or more modifications on uptake, tissue distribution, biodistribution, pharmacokinetics, pharmacodynamics, clearance, or cellular trafficking is assessed. In some embodiments, the modification is assessed by detecting the reporter in a mammal. In some embodiments, the reporter protein and/or the ASO is detected in a time course. In some embodiments, a modification is identified or selected that has a desired uptake, tissue distribution, biodistribution, pharmacokinetics, pharmacodynamics, clearance, or cellular trafficking. In some embodiments, the modification is assessed in a mammal. In some embodiments, the modification is assessed in a plurality of cells in cell culture in a high throughput manner.

In some embodiments, the ASO is covalently attached to a carrier molecule. In some embodiments, the effect of the carrier molecule on one or more features of the ASO is assessed. In some embodiments, attachment of a carbohydrate, a protein, a small molecule (e.g., a-tocopherol), a peptide (e.g., a cell-penetrating peptide), an antibody, a lipid (e.g., cholesterol) or a polymer (e.g., PEG) is assessed. In an embodiment, the ASO is not covalently attached to a carrier molecule.

### F. Kit

Also provided here are kits comprising a plurality of cells and/or a transgenic animal comprising the splice-switching ASO reporter system provided herein. In some embodiments, the kit comprises instructions for use comprising the methods provided herein. In some embodiments, the kit comprises instructions for detecting a functional reporter protein. In some embodiments, the kit comprises one or more ASOs.

### G. Sequences

TABLE 1
Modified HBB exon sequence

| SEQ ID | | |
|---|---|---|
| NO.: | SEQ ID | Base Sequence |
| 1 | Modified | gtgagtctatgggacgcttgatgttttc |
| | HBB Exon | tttccccttcttttctatggttaagttc |
| | | atgtcataggaaggggataagtaacagg |
| | | gtacagtttagaatgggaaacagacgaa |
| | | tgattgcatcagtgtggaagtctcagga |
| | | tcgttttagtttcttttatttgctgttc |
| | | ataacaattgttttcttttgtttaattc |
| | | ttgctttctttttttttcttctccgcaa |
| | | tttttactattatacttaatgccttaac |
| | | attgtgtataacaaaaggaaatatctct |
| | | gagatacattaagtaacttaaaaaaaaa |
| | | ctttacacagtctgcctagtacattact |
| | | atttggaatatatgtgtgcttatttgca |
| | | tattcataatctccctactttattttct |
| | | tttatttttaattgatacataatcatta |
| | | tacatatttatgggttaaagtgtaatgt |
| | | tttaatatgtgtacacatattgaccaaa |
| | | tcagggtaattttgcatttgtaatttta |
| | | aaaaatgctttcttcttttaatatactt |
| | | ttttgtttatcttatttctaatactttc |
| | | cctaatctctttctttcagggcaataat |
| | | gatacaatgtatcatgcctctttgcacc |
| | | attctaaagaataacagtgataatttct |
| | | gggttaaggtaagtgcaatatctctgca |
| | | tataaatatttctgcatataaattgtaa |
| | | ctgatgtaagaggtttcatattgctaat |
| | | agcagctacaatccagctaccattctgc |
| | | ttttattttatggttgggataaggctgg |
| | | attattctgagtccaagctaggcccttt |
| | | tgctaatcatgttcatacctcttatctt |
| | | cctcccacag |

TABLE 2
Primer and guide sequences

| SEQ ID | | |
|---|---|---|
| NO.: | SEQ ID | Base Sequence |
| 2 | ROSA26 Guide | ACTCCAGTCTTTCTAGAAGATGG |
| 3 | CAG fwd | CTTCCCTCGTGATCTGCAA |
| 4 | CAG probe | TTTCTAGGTAACCGATATCCCTGC |
| 5 | CAG rev | GCTATGAACTAATGACCCCGT |
| 6 | BGHpA_fwd | GGGGCTCGATCCTCTAGTTG |
| 7 | BGHpA_probe | CGCGCCGGCTAGAAGATGG |
| 8 | BGHpA_rev | ACCTGTTCAATTCCCCTGCA |
| 9 | DNA Cassette Amplicon 5′ fwd1 | GCCTTGGGTCATGTCCTACC |
| 10 | DNA Cassette Amplicon 3′ rev1 | TAGGGAGGTCGCAGTATCTG |
| 11 | DNA Cassette Amplicon 5′ fwd2 | AACAAGTGCTCCATGCTGGA |
| 12 | DNA Cassette Amplicon 3′ rev2 | CTTTAGAATGGTGCAAAGAGGC |
| 13 | DNA Cassette Amplicon 3′ fwd1 | ACCCGCTAAGAACCTCAAGATG |
| 14 | DNA Cassette Amplicon 3′_rev1 | TGGGAATTGAACTCAGGACTTC |
| 15 | DNA Cassette Amplicon 3′ fwd2 | GCCTCATCTACAACGTCAAGAT |
| 16 | DNA Cassette Amplicon 3′_rev2 | GTGCTCAGCAAGTCCTAGGG |

TABLE 3
ASO Sequences

| SEQ ID | | | |
|---|---|---|---|
| NO.: | SEQ ID | Base Sequence | Sugar Sequence |
| 17 | ssNC1 | GCAAATTCETATTCEC | LDLDLDLDLDLDLDLD |
| 18 | ssASO1 | AETTACCTTAAEECAG | LLDDLDDLLDDLLDLL |
| 19 | ssASO2 | GAGATATTGEACTTAEET | LDLLDLLDLLDDLDDLLL |
| 20 | ssASO3 | GAGATATTGEACTTAEET | LDLLDLLDLLDDLDDMLL |

E corresponds to 5-methyl C in the base sequences, and in the sugar sequence M is 2′-O-methoxyethylribose, L is LNA, and D is DNA. All backbones are phosphorothioate.

### EMBODIMENTS

Various embodiments of the methods, systems, articles of manufacture, and kits for treating muscle invasive bladder cancer in an individual provided herein are included in the following non-limiting list of embodiments.

1. A method of assessing delivery and/or splice-switching activity of an antisense oligonucleotide (ASO) comprising (i) introducing the ASO to a transgenic mouse, wherein the transgenic mouse comprises a plurality of cells which comprise a genetic sequence that is transcribed as a pre-mRNA, said pre-mRNA comprising a first segment encoding a first portion of a reporter protein, a cryptic sequence, and a second segment encoding a second portion of the reporter protein, wherein the ASO alters splicing of the pre-mRNA to exclude the cryptic sequence from a mature mRNA transcript, thereby causing expression of a functional form of the reporter protein, wherein the cryptic sequence comprises a sequence comprising at least 95% sequence identity to the nucleotide sequence set forth in SEQ ID NO.: 1 comprising nucleotide substitutions of T657G and A658T which result in decreased exclusion of the cryptic sequence in the absence of the ASO when compared to exclusion of the cryptic gene without the nucleotide substitutions of T657G and A658T; and (ii) detecting the expression of the reporter protein to assess the delivery and/or splice-switching activity of the ASO in the plurality of cells.

2. The method of embodiment 1, wherein an amount of ASO that is introduced to the transgenic mouse is about 50 μg or more, optionally wherein the amount of ASO introduced to the transgenic mouse is about 100 μg or more.

3. The method of any one of embodiments 1-2, wherein a concentration of ASO that is introduced to the transgenic mouse is about 0.5 μM or more, optionally wherein the concentration of ASO introduced to the transgenic mouse is about 1 μM or more, optionally wherein the concentration of ASO introduced to the transgenic mouse is about 2 μM or more, optionally wherein the concentration of ASO introduced to the transgenic mouse is about 3 μM or more, optionally wherein the concentration of ASO introduced to the transgenic mouse is about 4 μM or more, optionally wherein the concentration of ASO introduced to the transgenic mouse is about 5 μM or more, optionally wherein the concentration of ASO introduced to the transgenic mouse is about 10 μM or more, optionally wherein the concentration of ASO introduced to the transgenic mouse is about 15 μM or more, and optionally wherein the concentration of ASO introduced to the transgenic mouse is about 20 μM or more.

4. The method of any one of embodiments 1-3 wherein the sequence that is transcribed as a pre-mRNA is expressed ubiquitously in the transgenic mouse.

5. The method of any one of embodiments 1-3, wherein the sequence that is transcribed as a pre-mRNA is expressed in specific tissues or cells.

6. The method of embodiment 5, wherein the sequence that is transcribed as a pre-mRNA is expressed in neuronal cells, neurons, astrocytes, microglia, cortex, hippocampus, corpus collosum, interneuron, motor neuron, sensory neuron, microglia, astrocytes, brain, oligodendrocyte and/or neuronal tissues.

7. The method of any one of embodiments 1-6, wherein the ASO is introduced to the transgenic mouse by injection, inhalation, or intrathecal injection, and optionally wherein the ASO is administered directly to the central nervous system or the cerebrospinal fluid of the mammal.

8. The method of any one of embodiments 1-7, wherein the cryptic sequence comprises the nucleotide sequence set forth in SEQ ID NO.: 1.

9. A method of assessing delivery and/or splice-switching activity of an antisense oligonucleotide (ASO) comprising: (i) introducing the ASO to a plurality of cultured cells, wherein the plurality of cultured cells are isolated from a transgenic animal comprising a genetic sequence that is transcribed as a a pre-mRNA, said pre-mRNA comprising a first segment encoding a first portion of a reporter protein, a cryptic sequence, and a second segment encoding a second portion of the reporter protein, wherein the ASO alters splicing of the pre-mRNA to exclude the cryptic sequence from a mature mRNA transcript, thereby causing expression of a functional reporter protein, wherein the cryptic sequence comprises a sequence comprising at least 95% sequence identity to the nucleotide sequence of SEQ ID NO.: 1 comprising nucleotide substitutions of T657G and A658T which result in decreased exclusion of the cryptic sequence in the absence of the ASO when compared to exclusion of the cryptic gene without the nucleotide substitutions of T657G and A658T, and (ii) detecting the expression of the reporter protein to assess the delivery and/or activity of the ASO in the plurality of cultured cells.

10. The method of embodiment 9, wherein the transgenic animal is a mammal.

11. The method of embodiment 10, wherein the mammal is a mouse or a rat.

12. The method of any one of embodiments 9-11, wherein the cryptic sequence comprises the nucleotide sequence set forth in SEQ ID NO.: 1.

13. A method of assessing a feature of an antisense oligonucleotide (ASO) in a plurality of cells comprising: (i) providing the ASO to the plurality of cells, wherein the plurality of cells comprises a genetic sequence that is transcribed as a pre-mRNA, said pre-mRNA comprising a first segment encoding a first portion of a reporter protein, a cryptic sequence, and a second segment encoding a second portion of the reporter protein, wherein the ASO alters splicing of the pre-mRNA to exclude the cryptic sequence from a mature mRNA transcript, thereby causing expression of a functional reporter protein, wherein the cryptic sequence comprises one or more nucleotide insertions, deletions, or substitutions that result in decreased exclusion of the cryptic sequence in the absence of the ASO when compared to exclusion of the cryptic gene without said one or more nucleotide insertions, deletions, or substitutions, and (ii) detecting the expression of the reporter protein to assess a feature of the ASO in the plurality of cells.

14. The method of embodiment 13, wherein the cryptic sequence comprises a sequence of SEQ ID NO.: 1 comprising nucleotide substitutions of T657G and A658T which result in the decrease of the exclusion of the cryptic sequence in the absence of the ASO.

15. The method of embodiment 13 or embodiment 14, wherein the plurality of cells are mammalian cells.

16. The method of embodiment 15, wherein the mammalian cells are human, rat, or mouse cells.

17. The method of any one of embodiments 9-16, wherein the plurality of cells are from the same tissue.

18. The method of any one of embodiments 1-17, wherein the reporter protein is EGFP or luciferase.

19. The method of any one of embodiments 1-11 or 14-18, wherein the nucleotide substitutions of T657G and A658T decrease the exclusion of the cryptic sequence in the absence of splice-switching ASO by at least 10%, 20%, or 30%.

20. The method of any embodiments 1-19, wherein the ASO comprises a nucleotide sequence that is complementary to a part of the cryptic sequence, optionally wherein the ASO is complementary to a splice donor site or a splice acceptor site that regulates splicing of the cryptic sequence.

21. The method of any one of embodiments 1-20, wherein the reporter protein is detected in a living cell or animal.

22. The method of any one of embodiments 1-21, wherein the plurality of cells or the plurality of cultured cells comprise neuronal tissue, neurons, neurons, astrocytes, microglia, cortex, hippocampus, corpus collosum, interneurons, motor neuron, sensory neuron, microglia, astrocytes, and/or oligodendrocyte.

23. The method of any one of embodiments 1-22, wherein the ASO comprises one or more chemical modifications.

24. The method of embodiment 23, wherein the one or more chemical modifications comprise a modification to a phosphate backbone and/or a modification to a sugar, and optionally wherein, the chemical modification comprises 2-MOE, 2′-OMe, LNA, GalNAc, 5′ methylcytosine, a phosphorothioate bond, a phosphorodithioate bond, an alkylphosphonate bond, a phosphoroamidate bond, a boranophosphate bond, and/or a morpholino ring.

25. The method of any one of embodiments 1-24, wherein the ASO is 12 to 30 nucleotides in length, optionally wherein the ASO is 14 to 18 nucleotides in length, and optionally wherein the ASO is 16 nucleotides in length.

26. The method of any one of embodiments 9-25, wherein an amount of ASO that is introduced to the plurality of cultured cells is about 1 μg or more, optionally wherein the amount of ASO introduced to the plurality of cultured cells is about 10 μg or more, optionally wherein the amount of ASO introduced to the plurality of cultured cells is about 50 μg or more, optionally wherein the amount of ASO introduced to the plurality of cultured cells is about 100 μg or more, optionally wherein the amount of ASO introduced to the plurality of cultured cells is about 150 μg or more, and optionally wherein the amount of ASO introduced to the plurality of cultured cells is about 200 μg or more.

27. The method of any one of embodiments 9-25, wherein a concentration of ASO that is introduced to the plurality of cells or the plurality of cultured cells is about 0.5 μM or more, optionally wherein the concentration of ASO introduced to the plurality of cultured cells is about 1 μM or more, optionally wherein the concentration of ASO introduced to the plurality of cultured cells is about 2 μM or more, optionally wherein the concentration of ASO introduced to the plurality of cultured cells is about 3 μM or more, optionally wherein the concentration of ASO introduced to the plurality of cultured cells is about 4 M or more, and optionally wherein the concentration of ASO introduced to the plurality of cultured cells is about 5 μM or more.

28. The method of any one of embodiments 1-27, wherein the detected expression of the reporter is indicative of presence, amount, and/or splice-switching activity of the ASO in the cell.

29. The method of any one of embodiments 1-28, wherein the feature is a formulation, a clearance rate, a delivery route, pharmacokinetics, pharmacodynamics, uptake, tissue localization, concentration, cellular internalization, impact on gene expression, a modification, impact of genetics on ASO activity, cellular trafficking, or biodistribution.

30. The method of any one of embodiments 13-29, wherein providing the ASO to the cell comprises providing the ASO to a cell in a cell culture, optionally wherein the cell is a mammalian cell, and optionally wherein the cell is a human cell, a rat cell, or a mouse cell.

31. The method of any one of embodiments 13-30, wherein the ASO is provided to a mammal comprising the plurality of cells, optionally wherein the ASO is administered to the animal by injection or by intrathecal injection, and optionally wherein the ASO is administered directly to the central nervous system or the cerebrospinal fluid of the mammal.

32. The method of any one of embodiments 1-31, wherein the plurality of cells or the comprises a DNA cassette comprising the genetic sequence.

33. The method of embodiment 32, wherein the DNA cassette is integrated into the genome of the cell at a safe harbor locus.

34. The method of embodiment 33, wherein the DNA cassette is integrated at the ROSA26 locus.

35. The method of any one of embodiments 32-34, wherein the DNA cassette further comprises a polynucleotide encoding a second reporter protein.

36. The method of embodiment 35, wherein the second reporter protein is a fluorescent or luminescent protein.

37. The method of embodiment 35 or embodiment 36, wherein the second reporter protein is not EGFP or luciferase.

38. The method of embodiment 37, wherein the second reporter protein is mKate.

39. The method of any one of embodiments 32-38, wherein the DNA cassette comprises a promoter operably linked to the nucleic acid that is transcribed as the pre-mRNA.

40. The method of embodiment 39, wherein the promoter is operably linked to the first or second reporter protein.

41. The method of embodiment 39 or embodiment 40, wherein the promoter is constitutive.

42. The method of embodiment 39 or embodiment 40, wherein the promoter is inducible.

43. The method of any one of embodiments 39-42, wherein the promoter is tissue-specific.

44. The method of embodiment 43, wherein the promoter is specific to neuronal cells.

45. The method of any one of embodiments 39-44, wherein the promoter is a ubiquitous promoter.

46. The method of embodiment 45, wherein the promoter is a CMV early enhancer/chickenβ-actin (CAG) promoter.

47. The method of any one of embodiments 1-46, wherein the splice-switching ASO is complementary to a 5′ splice donor site of the cryptic sequence.

48. The method of any one of embodiments 1-47, wherein the cryptic sequence is a cryptic β-globin (HBB).

49. The method of embodiment 48, wherein the cryptic sequence comprises the sequence set forth in SEQ ID NO: 1.

50. The method of embodiment 49, wherein the splice-switching ASO is complementary to SEQ ID NOs: 17-20.

51. The method of any one of embodiments 1-50, comprising testing a plurality of splice-switching ASOs.

52. The method of embodiment 51, wherein each of the plurality of splice-switching ASOs comprises the same nucleotide sequence.

53. The method of embodiment 51 or embodiment 52, wherein each of the plurality of splice-switching ASOs comprises a nucleotide sequence that is complementary to the cryptic exon.

54. The method of any one of embodiments 51-53, wherein the plurality of splice-switching ASOs differ with respect to formulation, chemical modification, delivery method, concentration, or amount.

55. The method of any one of embodiments 51-54, wherein the plurality of splice-switching ASOs are assessed in a high-throughput system.

56. The method of embodiment 55, wherein the high-throughput system comprises one or more automated processes.

57. The method of embodiment 55 or embodiment 56, wherein the high throughput system comprises cell culture and/or detection of the luciferase and/or EGFP in a 96 well or 364 well format.

58. A kit comprising a transgenic mouse comprising a plurality of cells which comprise a genetic sequence that is transcribed as a pre-mRNA which comprises a first segment encoding a first portion of a reporter protein, a cryptic sequence, and a second segment encoding a second portion of the reporter protein; or a plurality of cells which comprise a genetic sequence that is transcribed as a pre-mRNA which comprises a first segment encoding a first portion of a reporter protein, a cryptic sequence, and a second segment encoding a second portion of the reporter protein; and instructions for assessing a feature of an ASO by detecting the reporter protein according to the method of any one of claims 1-57.

59. The kit of embodiment 58, further comprising a plurality of ASOs.

### EXAMPLES

The following examples are included for illustrative purposes only and are not intended to limit the scope of the present disclosure.

### Example 1: Evaluation of a EGFP Splice-Switching ASO Activity Reporter in HEK293 Cells

To quantify ASO activity quickly and easily, a reporter construct was developed that measures EGFP mRNA splicing correction by ASOs (FIG. 1A). The reporter construct splits the EGFP coding sequence in two parts with an internal exon comprising β-globin (HBB) containing a C to T mutation at nucleotide (nt) 1125. This mutation activates cryptic 5′ and 3′ splice sites that, in the absence of ASO, are used preferentially over the unaltered EGFP sites and thus prevents functional EGFP expression (FIG. 1A and Table 4). However, the cryptic 5′ splice site is sterically blocked upon ASO uptake and activity, promoting EGFP mRNA splice-switching and induction of EGFP fluorescence. While this model has proven to be effective in measuring ASO activity, the construct is inherently leaky with 26.1% of HEK293 cells expressing the reporter showing background EGFP signal (FIG. 1B, left). Thus, to reduce EGFP background, the HBB exon sequence was modified at position 1128-1129 from TA to GT (Table 4). Compared to the unmodified construct, these mutations increased the strength of the aberrant splice sites making these locations preferred under non-treated conditions and effectively reduced EGFP leakiness in HEK293 cells from 26.1% to 3.38% (FIG. 1B).

Given the improved splice site strength in the modified EGFP reporter construct (ssEGFP.HBB), ASO-mediated EGFP splice-switching activity was tested and validated in cells. A construct was generated containing both mKate2 and ssEGFP.HBB under the control of the CMV early enhancer/chicken 3-actin (CAG) promoter. Upon CRE removal of the loxp cassette, mKate2 is constitutively expressed and ASO activity can be monitored by the upregulation of EGFP expression and fluorescence intensity (FIG. 1C). To test the efficacy of this construct, a pooled stable cell line was generated using piggyBac transposase in a HEK293 cell line containing a stably expressing nuclear localization sequence (NLS)-CRE using fluorescence activated cell sorting (FACS) for mKate2 positive cells over the course of one month (FIG. 1D). After a pooled cell line was obtained, the induction of EGFP was tested after lipofectamine transfection of four splice-switching ASOs (ssASOs). The stable HEK293 cells expressed mKate2 broadly. Furthermore, while treatment with a splice-switching negative control ASO (ssNC1) did not promote EGFP expression, ssASOs 1-3 induced robust EGFP fluorescence two days after transfection (FIG. 1E). These data validated the ASO-dependent activity of the ssEGFP.HBB cassette and prompted the development of a mouse model harboring the improved transgene.

TABLE 4
ssEGFP.HBB Sequence

| Position | ssEGFP.HBB Sequence (1570 bp DNA) SEQ ID NO.: 21 |
|---|---|
| 1 | atggtgagca agggcgagga gctgttcacc ggggtggtgc ccatcctggt cgagctggac |
| 61 | ggcgacgtaa acggccacaa gttcagcgtg tccggcgagg gcgagggcga tgccacctac |
| 121 | ggcaagctga ccctgaagtt catctgcacc accggcaagc tgcccgtgcc ctggcccacc |
| 181 | ctcgtgacca ccctgaccta cggcgtgcag tgcttcagcc gctaccccga ccacatgaag |
| 241 | cagcacgact tcttcaagtc cgccatgccc gaaggctacg tccaggagcg caccatcttc |
| 301 | ttcaaggacg acggcaacta caagacccgc gccgaggtga agttcgaggg cgacaccctg |
| 361 | gtgaaccgca tcgagctgaa gggcatcgac ttcaaggagg acggcaacat cctggggcac |
| 421 | aagctggagt acaactacaa cagccacaac gtctatatca tggccgacaa ggtgagtcta |
| 481 | tgggacgctt gatgttttct ttccccttct tttctatggt taagttcatg tcataggaag |
| 541 | gggataagta acagggtaca gtttagaatg ggaaacagac gaatgattgc atcagtgtgg |
| 601 | aagtctcagg atcgttttag tttcttttat ttgctgttca taacaattgt tttcttttgt |
| 661 | ttaattcttg ctttcttttt ttttcttctc cgcaattttt actattatac ttaatgcctt |
| 721 | aacattgtgt ataacaaaag gaaatatctc tgagatacat taagtaactt aaaaaaaaac |
| 781 | tttacacagt ctgcctagta cattactatt tggaatatat gtgtgcttat ttgcatattc |
| 841 | ataatctccc tactttattt tcttttattt ttaattgata cataatcatt atacatattt |
| 901 | atgggttaaa gtgtaatgtt ttaatatgtg tacacatatt gaccaaatca gggtaatttt |
| 961 | gcatttgtaa ttttaaaaaa tgctttcttc ttttaatata cttttttgtt tatcttattt |
| 1021 | ctaatacttt ccctaatctc tttctttcag ggcaataatg atacaatgta tcatgcctct |
| 1081 | ttgcaccatt ctaaagaata acagtgataa tttctgggtt aaggtaagtg caatatctct |
| 1141 | gcatataaat atttctgcat ataaattgta actgatgtaa gaggtttcat attgctaata |
| 1201 | gcagctacaa tccagctacc attctgcttt tattttatgg ttgggataag gctggattat |
| 1261 | tctgagtcca agctaggccc ttttgctaat catgttcata cctcttatct tcctcccaca |
| 1321 | gcagaagaac ggcatcaagg tgaacttcaa gatccgccac aacatcgagg acggcagcgt |
| 1381 | gcagctcgcc gaccactacc agcagaacac ccccatcggc gacggccccg tgctgctgcc |
| 1441 | cgacaaccac tacctgagca cccagtccgc cctgagcaaa gaccccaacg agaagcgcga |
| 1501 | tcacatggtc ctgctggagt tcgtgaccgc cgccgggatc actctcggca tggacgagct |
| 1561 | gtacaagtaa |

### Example 2: ssEGFP.HBB ROSA26 Knock-In Mice Show Copy Number-Dependent Transgene Expression

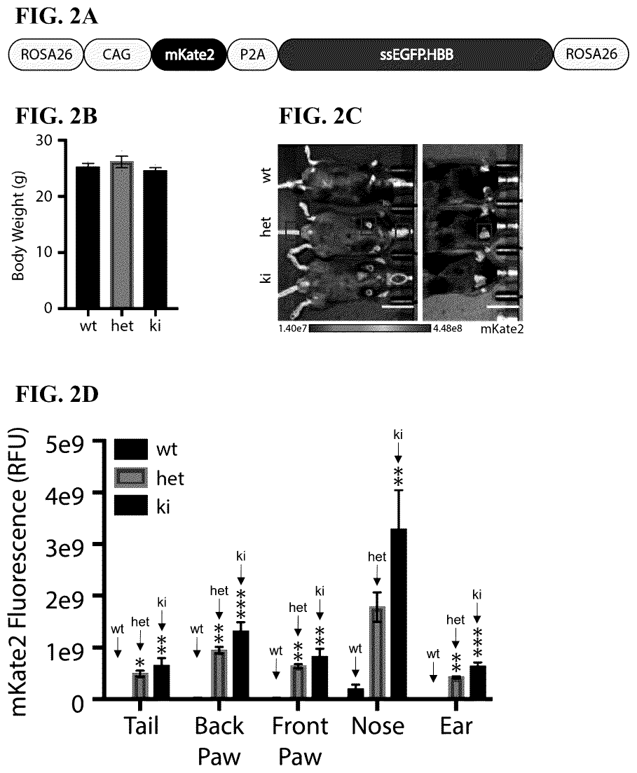

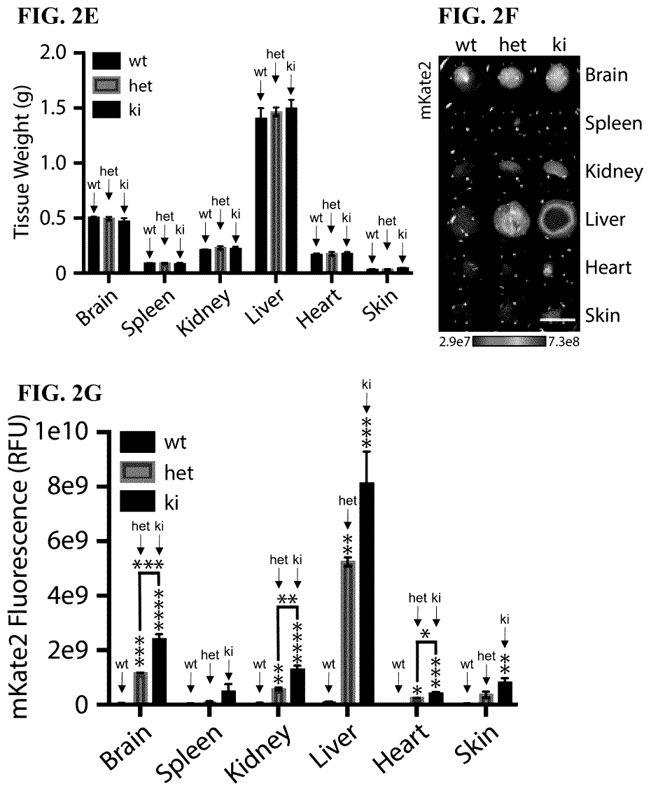

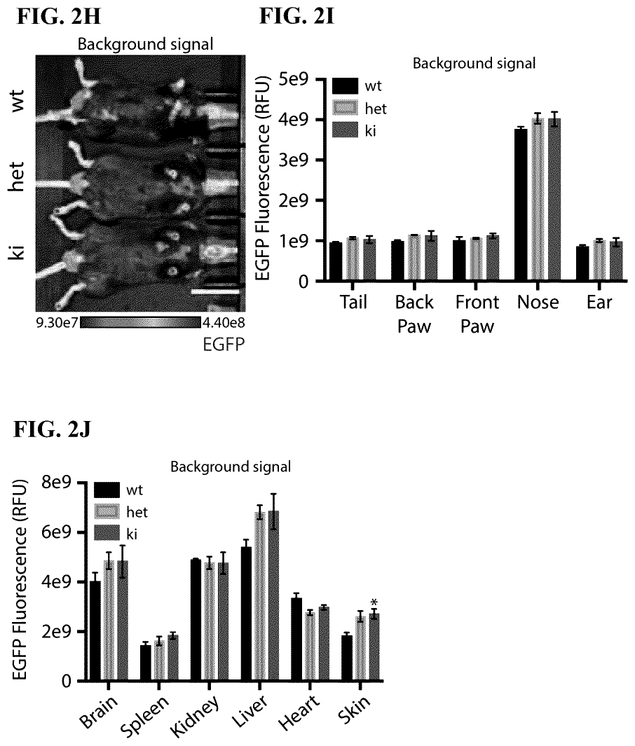

A transgenic mouse model was generated harboring the ssEGFP.HBB cassette to quantify ASO activity in relevant in vitro primary cell culture model systems and in vivo. To quickly assess prospective founder animals, the loxp cassette was removed from the previously characterized transgene (FIG. 1C vs. FIG. 2A) and CRISPR/Cas9 was used to insert the construct into the ROSA26 safe-harbor locus. G0 mosaic animal sequencing confirmed the generation of three sequence-positive ssEGFP.HBB founders, one of which contained a single-insertion. This sequence-positive, single-insertion animal was successfully bred with C57BL/6J mice and the cohort was transferred into production for further characterization. Animals bred within this colony produce litters with Mendelian genotype ratios with consistent pre- and post-wean mortality rates across genotypes. Furthermore, animal body weight is also maintained (FIG. 2B), which when taken together suggests there are no overt toxicities in these mice. Transgene expression levels were then analyzed in wt, het, and ki animals (FIGS. 2C-2G). As mKate2 is constitutively expressed under a CAG promoter, mKate2 fluorescence was first quantified in the body parts of live animals revealing a copy number-dependent increase in fluorescence intensity (FIGS. 2C-2D). Furthermore, while ex vivo tissue weight was not affected (FIG. 2E), mKate2 tissue fluorescence also increased concurrently with transgene copy number in the brain, spleen, kidney, liver, heart, and skin as measured in the ear (FIGS. 2F-2G). The background EGFP fluorescence was analyzed via both in vivo and ex vivo fluorescence imaging (FIGS. 211-2J). There was no significant change in EGFP fluorescence across genotypes suggesting any observed signal was due to tissue autofluorescence. Together, these data show that the ssEGFP.HBB transgene is widely expressed across all tissues and thus can be used to evaluate ASO activity.

### Example 3: EGFP Induction is ssASO Incubation Time- and Concentration-Dependent in Primary Neurons, Microglia, and Astrocytes

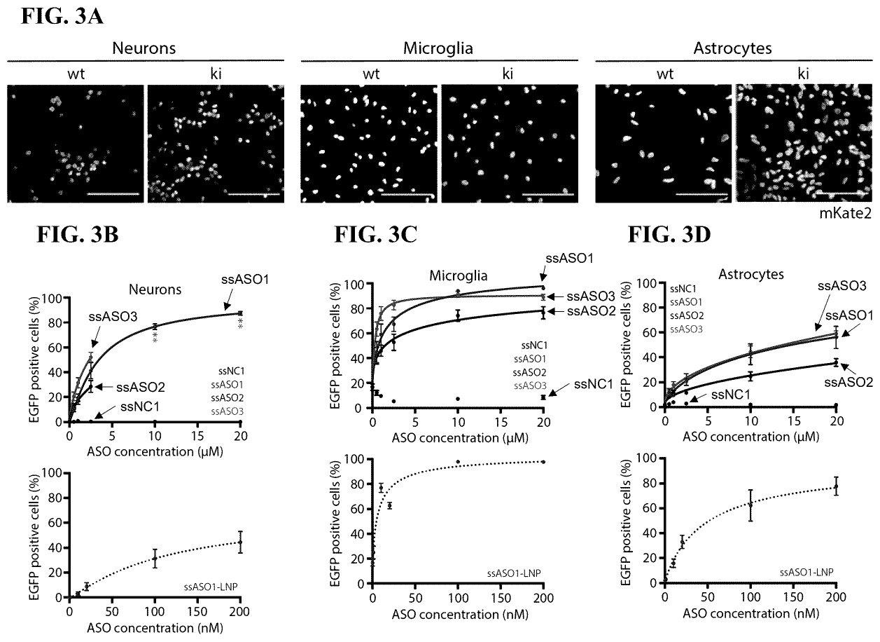

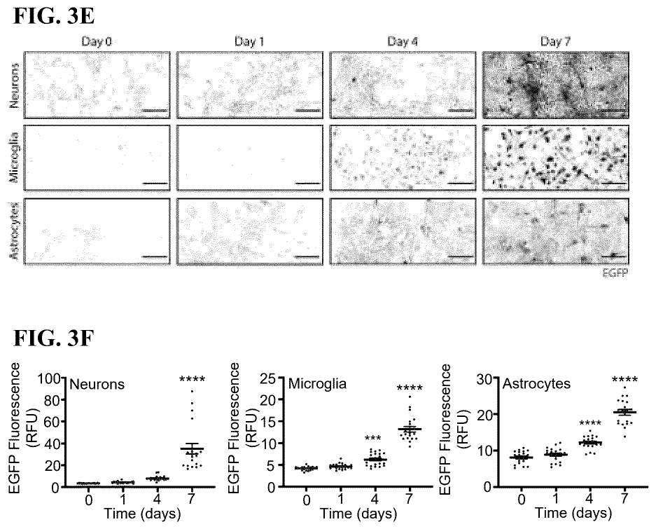

Given the rapid development of ASO therapeutics for the treatment of neurodegenerative diseases, transgene expression and ssASO activity was analyzed in primary CNS cell cultures to assess the in vitro utility of the ssEGFP.HBB mouse model. Ki cortical neurons, microglia, and astrocytes showed robust mKate2 expression as compared to cultures obtained from wt animals (FIG. 3A). These data are consistent with the in vivo and ex vivo tissue imaging studies (FIGS. 2A-2J), revealing mKate2 fluorescence is a robust marker of transgene expression across unique assays, tissues, and cell types. As all cell types analyzed robustly expressed mKate2, ASO-mediated EGFP splice-switching was further characterized (FIGS. 3A-3D). Cells were treated with increasing concentrations of non-encapsulated or lipid nanoparticle (LNP)-encapsulated ssASO, and the percent of EGFP positive-cells was calculated after three days of treatment using EGFP-DAPI colocalization (FIGS. 3B-3D). Cortical neurons, microglia, and astrocytes all showed an ASO concentration-dependent induction of EGFP fluorescence that was specific for active ssASOs 1-3, but not ssNC1, suggesting ASOs must actively target the 5′ cryptic splice site to induce fluorescence. Furthermore, while ASO toxicity was not noted in microglia or astrocytes (FIGS. 3C-3D), neuronal cultures were particularly susceptible to ssASO2 and 3 toxicity, with signs of death observed at ASO concentrations as low as 1 μM (FIG. 3B, stars). LNP encapsulation enhanced EGFP splice-switching activity in all cell types studied, suggesting LNPs broadly increase ASO delivery efficacy to primary brain cells (FIGS. 3B-3D, bottom). Given ssASO1 was well-tolerated in all cell types, EGFP fluorescence intensity was quantified over the course of 7 days (FIGS. 3E-3F). Cells were treated with 10 μM ssASO1, fixed at defined time points, and EGFP signal was quantified by normalizing total EGFP intensity to the number of DAPI+ cells per field of view. Interestingly, while glial cells showed a significant increase in EGFP intensity 4 days following treatment, neuronal EGFP induction was slower with a significant change in signal not detected until 7 days post dosing (FIG. 3F). These data show that EGFP splice switching in primary cell cultures is dependent on both ASO concentration and incubation time, and provide a novel quantitative, high-throughput method of examining ASO activity in cell culture systems.

### Example 4: Intracerebroventricular Injection Promotes Widespread ASO Internalization and Time-Dependent EGFP Splice-Switching in the Brain

ASO-mediated EGFP splice-switching was next evaluated in ssEGFP.HBB transgenic ki male mice following a single intracerebroventricular (ICV) administration of saline or ssASO1 (100 or 200 μg final dose) (FIG. 4A). ASO-treated mice showed dose-dependent acute side effects including lethargy immediately following injection (FIG. 4B). However, the mice recovered well with normal activity resumed within 2-4 hours (hrs) post-surgery and minimal weight loss observed over the course of the experiment (FIG. 4C). Additionally, nominal ASO-mediated brain gliosis, as measured using IBA1 and GFAP immunohistochemistry (IHC), was observed post ICV injection, suggesting ssASO1 was largely well tolerated (FIGS. 4D-4F). To examine ASO splice-switching dynamics in vivo, mKate2/EGFP fluorescence 7 and 14 days following ssASO1 ICV injection (100 μg) was quantified. Fluorescence imaging in live animals revealed an increase in EGFP signal specifically through the skull (FIGS. 4G-4I). These data did not reach significance, however, likely due to the variation in hair regrowth over the course of the experiment and skin pigmentation between animals. Given the surprising increase in EGFP fluorescence in live animals, ex vivo tissue fluorescence imaging was performed on harvested organs including the brain, spleen, kidney, liver, heart, and skin as measured in the ear (FIG. 4J). mKate2 fluorescence intensity was consistent across all treatment groups and organs analyzed (FIGS. 4K-4M). However, ex vivo whole-brain fluorescence imaging revealed a time-dependent increase in EGFP intensity from 7 to 14 days post-dosing (FIGS. 4N-4P). EGFP fluorescence intensity in the brain was dependent on both the concentration of ASO administered and time post dosing (FIG. 4O). EGFP signal in the brain was similar for both WT and saline-injected mKate2.ssEGFP.HBB ki animals, suggesting the observed fluorescence in these mice was due to tissue autofluorescence and not EGFP construct leakiness. There was also a trend showing an increase in EGFP signal in the liver over the course of the experiment (FIG. 4P).

As the brain had a strong, significant increase in EGFP, we next utilized immunohistochemistry (IHC) to further evaluate ASO localization and EGFP splice-switching activity (FIGS. 4Q-4R). All ssASO1-treated animals showed broad anti-ASO staining in the brain that was largely maintained throughout the duration of the experiment. Consistent with the ex vivo whole tissue EGFP fluorescence analysis, ssASO1 internalization correlated with a significant increase in EGFP signal in the brain both 7 and 14 days post-ASO injection. These data also revealed that ASOs are highly stable in the tissue once internalized. Furthermore, ASO accumulation in the brain was concentration dependent. Consistent with our ex vivo whole-tissue EGFP fluorescence analysis, we observed a significant increase in brain EGFP signal both 7- and 14-days post injection as compared to saline-treated animals (FIGS. 4Q, 4R). The induction of EGFP fluorescence was ssASO1 concentration-dependent across the entire brain and in the cortex, hippocampus, and thalamus (FIGS. 4Q-4V). A strong dose-dependent colocalization of EGFP and ASO staining was also observed, suggesting EGFP splice-switching results from productive ASO internalization (Figure S4B). However, brain tissue may be differentially susceptible to ASO-mediated splice-switching, as percentage EGFP area did not directly correlate with ASO-EGFP colocalization across the brain regions analyzed (FIGS. 4S-4V). Together, these data reveal that ASO uptake correlates with EGFP splice-switching in the brain, which can be easily monitored over time.

### Example 5: EGFP Splice-Switching in the Brain is ASO-Dose Dependent Following ICV Injection

An ASO dose response was also assessed 14 days following ICV injection of saline or ssASO1 (100 μg or 200 μg). Fluorescence imaging in live animals revealed that while mKate2 fluorescence intensity was unaffected by ssASO1 administration, EGFP splice-switching was induced ASO dose-dependently in the brain, however the data did not reach statistical significance (FIG. 5A-5D). Fluorescence intensity was next assessed in ex vivo harvested organs. While tissue weight and mKate2 fluorescence were maintained across treatment groups as well as unique organs (FIG. 5E-5H). EGFP fluorescence was significantly increased in the brain following ssASO1 administration (FIG. 5I). The liver also showed a ssASO1-mediated dose-response (FIG. 5J). Given the significant increase in EGFP intensity in the brain at both doses, ASO localization and EGFP fluorescence was next assessed using IHC (FIG. 5K-5L). Increasing ASO dosing from 100 to 200 μg significantly increased ASO internalization into the brain. However, there was not a concomitant increase in EGFP signal as only a modest increase in fluorescence intensity was observed. These data suggest that ICV injecting ssASO1 at doses greater than 100 μg does not greatly affect splice-switching activity in the brain.

### Example 6: ICV Injection of ssASO1 Induces EGFP Splice-Switching in Neurons, Microglia, Astrocytes, and Oligodendrocytes

As all ssASO1-treated groups showed increased EGFP expression following ICV injection, but which CNS cell types are susceptible to ASO-mediated EGFP splice-switching in vivo was still to be determined. Brain sections were stained with cell type-specific antibodies labeling neurons (NeuN), microglia (Iba1), astrocytes (S100B), or oligodendrocytes (Olig2), and EGFP colocalization was assessed using immunofluorescence (FIG. 6A-6H). EGFP expression was observed in neurons, microglia, and astrocytes in both the cortex and hippocampus in all conditions studied, and EGFP-positive oligodendrocytes were abundant in the corpus callosum (FIG. 6A-6D), white arrows). Together, these data revealed that CNS cell types across the brain internalize ssASOs and promote EGFP splice-switching. Given the robust EGFP expression observed, the degree of EGFP colocalization was quantified with these cell type-specific markers using Mander's coefficients that range from zero (no colocalization) to one (full colocalization) (FIG. 6I). All cell types in each brain region showed colocalization (M>0). Interestingly, there was no significant difference detected between groups treated with ASO for 7 or 14 days, or between ASO dosing concentrations for any of the cell types or brain regions analyzed. This could be due to animal-to-animal variation or differences in cellular EGFP intensity at a given time point or dose, which is not accounted for in this analysis. EGFP colocalization with cell-type-specific markers were evaluated by quantifying the absolute number of cells double positive for EGFP and cell-type-specific antibody staining, where cell nuclei were used as a proxy for individual cells (FIG. 6J). These data revealed that all major CNS cell types are susceptible to EGFP splice-switching, which is ASO-dependent. In particular, both neuronal and astrocyte populations had the greatest EGFP co-localization. Taken together, these data demonstrate a robust readout of ASO uptake and activity across cell types as well as regions in the brain, which provide a foundational tool to further analyze ASO biology.

### Example 7: LNP Encapsulation Increases ASO Efficacy in mKate2.ssEGFP.HBB-Expressing HEK293 Cells

Currently, studying LNP efficacy is limited by the inability to easily assess cargo activity. Thus, as a proof of concept, ASO-LNP activity and biodistribution was screened in vitro as well as in vivo using the mKate2.ssEGFP.HBB model. Microfluidic mixing was utilized to encapsulate either negative control ssNC1 or ssASO1 in LNPs containing lipid combinations (Formulations 1-4, Table 5) reported to differentially distribute to unique cell types and/or tissues (Byrnes, A. E., et al. (2023). Mol. Ther. Nucleic Acids 32, 773-793). All particles behaved well post mixing with consistent diameters and polydispersity indices measured using dynamic light scattering. The formulations were then screened for EGFP splice-switching activity in mKate2.ssEGFP.HBB-expressing HEK293 cells and a dose-dependent increase in EGFP positive cells was observed for all tested ssASO1-containing formulations (FIG. 7A). While free ASO up-take did not dramatically increase the percentage EGFP+ cells after 3 days of treatment, dose-dependent EGFP splice-switching activity was observed for all formulations containing ssASO1 (FIG. 7B). ssNC1-LNPs showed little splice-switching activity across the concentrations examined, revealing that EGFP induction is dependent on active ASO encapsulation. Signs of toxicity were noted with formulations 3 and 4 when dosed at high ASO concentrations (500 nM). However, both ssNC1- and ssASO1-LNPs showed similar signs of cell clumping, suggesting this toxicity was not ASO specific and thus was likely due to the lipid mixtures. Given these formulations had favorable physicochemical properties and enhanced EGFP splice-switching in cells with only moderate toxicity observed, ASO-LNP delivery was further characterized in mice.

TABLE 5
ssEGFP.HBB Sequence

| Formu- | | | | Dia- | Polydis- |
|---|---|---|---|---|---|
| lation | Lipid | | ASO | meter | persity |
| Number | Composition | Target | | (nm) | (%) |
| 1 | SM-102: | Liver | ssASO1 | 68.9 | 10.6 |
| | DSPC:DMG- | | ssNC1 | 74.1 | 14.8 |
| | PEG2K: | | | | |
| | Cholesterol | | | | |
| | (50:10:2:38) | | | | |
| 2 | MC3: | Unknown | ssASO1 | 72.9 | 9.8 |
| | DSPC:DMG- | | ssNC1 | 74.1 | 9.3 |
| | PEG2K: | | | | |
| | Cholesterol | | | | |
| | (40:10:2:48) | | | | |
| 3 | DOTAP: | Lung | ssASO1 | 142.4 | 15.9 |
| | MC3:DPSC: | | ssNC1 | 155.5 | 15.3 |
| | DMG- | | | | |
| | PEG2K: | | | | |
| | Cholesterol | | | | |
| | (50:25:5:0.8: | | | | |
| | 19.2) | | | | |
| 4 | DOTAP: | Lung | ssASO1 | 72.7 | 4.3 |
| | cKK-E12:C16 | | ssNC1 | 72.2 | 9.4 |
| | PEG2K: | | | | |
| | Cholesterol | | | | |
| | (12.5:50:2.5:35) | | | | |

### Example 8: Liver- and Lung-Targeting ssASO1-LNPs are Dose-Dependently Active in mKate2.ssEGFP.HBB HEK293 Cells

An i.v. injection of either saline or a pooled mixture of ASO-LNPs (Formulations 1-4, Table 5) was next administered in 1- to 3-month-old mixed-gender mKate2.ssEGFP.HBB ki mice and the animals were assessed over 20 days (FIG. 8A). No acute toxicity was observed in the hours post injection and animals gained weight over the course of the experiment, suggesting the pooled ASO-LNPs were well tolerated (FIG. 8B). To examine ASO-LNP activity and biodistribution, mKate2 and EGFP fluorescence was quantified in ex vivo tissues both 6 and 20 days post dosing (FIG. 8C-8E). mKate2 fluorescence was largely maintained in the brain, spleen, kidneys, heart, liver, and lungs (FIG. 8D). In contrast, EGFP intensity increased specifically in the liver of mice injected with pooled ASO-LNPs (FIG. 8E). Liver EGFP signal was consistent between 6 and 20 days post dosing, revealing that ASO-LNP-mediated splice-switching is robust over time. However, despite administering a pooled mixture of ASO-LNPs that were previously reported to differentially distribute to the liver or lungs, EGFP signal in the lungs did not change over the course of the experiment. There was also no significant increase in EGFP intensity in the brain, spleen, kidneys, or heart. Given the robust induction of EGFP in the liver, we next aimed to isolate EGFP+ cells (FIG. 8F). Liver single-cell suspensions were prepared from WT animals and ki littermates injected with saline or pooled ASO-LNP 6 days after dosing. FACS analysis revealed a clear enhancement in EGFP+ liver cells when isolated from ASO-LNP-treated mKate2.ssEGFP.HBB ki animals. There were two main populations of EGFP+ cells observed, suggesting multiple cell types are susceptible to ASO-mediated splice-switching in the liver (FIG. 8F). Together, these data show that EGFP splice-switching can be monitored in peripheral tissues and also provide a foundation for assessing EGFP signal using FACS in future studies.

### Example 9: Quantifying ASO Activity in HEK293 Cells Using a Luciferase Splice-Switching Reporter

To facilitate rapid, sensitive quantification of ASO activity, a luciferase reporter was developed that measures Luc2 mRNA splicing correction by ASOs (FIG. 9A). The Luc2 mRNA coding sequence was split in two parts with an internal β-globin (HBB) exon, leading to the disruption of the Luc2 reading frame. Correct Luc2 mRNA splicing and translation should be restored by ASOs specifically designed to sterically block a cryptic HBB splice site, thus providing a positive bioluminescence readout for ASO delivery as well as activity. Sequence information for the ssLuc2.HBB reporter cassette is provided in Table 5.

A vector was generated that contained a far-red fluorescent protein, mKate2, and ssLuc2.HBB under the control of the CMV early enhancer/chicken 3-actin (CAG) promoter to enable the simultaneous assessment of both construct expression and ASO activity. A loxP-stop-loxP (LSL) cassette was also included upstream of these two reporter genes to ensure expression was Cre-driven (FIG. 9B). To test the utility of this construct, a stable cell line was created by co-transfecting piggyBac transposase and the reporter vector in HEK293 cells containing a nuclear localization sequence (NLS)-Cre. mKate2+ cells were then sorted over the course of ~1 month using fluorescence activated cell sorting (FACS) until a pooled stable cell line was obtained (mKate2.ssLuc2.HBB; FIG. 9C). The mKate2.ssLuc2.HBB HEK293 cells expressed mKate2 while non-transfected wild-type (wt) cells lacked fluorescence signal (FIG. 9D-9E). Given the robust mKate2 expression in these cells, ASO-mediated Luc2 splice-switching activity (FIG. 9F-9G) was analyzed. Cells were treated with increasing concentrations of splice-switching ASOs (ssASOs) and measured bioluminescence signal after three days of treatment. While non-treated mKate2.ssLuc2.HBB cells had a moderate increase in background bioluminescence after addition of 50 μM D-luciferin as compared to wt cells (FIG. 911), a significant ASO-dependent increase in bioluminescence was observed in cells treated with ssASO1-3 (FIG. 9F). In contrast, no dramatic increase in bioluminescence was seen in cells treated with negative control ASO, ssNC1. These data suggest that Luc2 splice-switching is ASO concentration-dependent. As D-luciferin concentration also affects bioluminescence intensity, cells were next treated with 500 nM ASO for three days and assessed bioluminescence signal after adding increasing concentrations of D-luciferin (FIG. 9G). Luc2 bioluminescence was substrate concentration-dependent in all active ASO-treated cells. mKate2 fluorescence intensity remained unchanged during both experiments (FIG. 9I-9J) suggesting ASO treatment did not affect cell health or construct expression. Together, these data validated the ASO-dependent splice-switching of the ssLuc2.HBB cassette and revealed that bioluminescence is a sensitive readout for ASO activity, prompting the creation of a mouse model harboring these reporters.

TABLE 6
ssLuc2.HBB Sequence

| Position | ssLuc2.HBB Sequence (2503 bp DNA) SEQ ID NO.: 22 |
|---|---|
| 1 | atggaagatg ccaaaaacat taagaagggc ccagcgccat tctacccact cgaagacggg |
| 61 | accgccggcg agcagctgca caaagccatg aagcgctacg ccctggtgcc cggcaccatc |
| 121 | gcctttaccg acgcacatat cgaggtggac attacctacg ccgagtactt cgagatgagc |
| 181 | gttcggctgg cagaagctat gaagcgctat gggctgaata caaaccatcg gatcgtggtg |
| 241 | tgcagcgaga atagcttgca gttcttcatg cccgtgttgg gtgccctgtt catcggtgtg |
| 301 | gctgtggccc cagctaacga catctacaac gagcgcgagc tgctgaacag catgggcatc |
| 361 | agccagccca ccgtcgtatt cgtgagcaag aaagggctgc aaaagatcct caacgtgcaa |
| 421 | aagaagctac cgatcataca aaagatcatc atcatggata gcaagaccga ctaccagggc |
| 481 | ttccaaagca tgtacacctt cgtgacttcc catttgccac ccggcttcaa cgagtacgac |
| 541 | ttcgtgcccg agagcttcga ccgggacaaa accatcgccc tgatcatgaa cagtagtggc |
| 601 | agtaccggat tgcccaaggg cgtagcccta ccgcaccgca ccgcttgtgt ccgattcagt |
| 661 | catgcccgcg accccatctt cggcaaccag atcatccccg acaccgctat cctcagcgtg |
| 721 | gtgccatttc accacggctt cggcatgttc accacgctgg gctacttgat ctgcggcttt |
| 781 | cgggtcgtgc tcatgtaccg cttcgaggag gagctattct tgcgcagctt gcaagactat |
| 841 | aagattcaat ctgccctgct ggtgcccaca ctatttagct tcttcgctaa gagcactctc |
| 901 | atcgacaagt acgacctaag caacttgcac gagatcgcca gcggcggggc gccgctcagc |
| 961 | aaggaggtag gtgaggccgt ggccaaacgc ttccacctac caggcatccg ccagggctac |
| 1021 | ggcctgacag aaacaaccag cgccattctg atcacccccg aaggggacga caagcctggc |
| 1081 | gcagtaggca aggtggtgcc cttcttcgag gctaaggtgg tggacttgga caccggtaag |
| 1141 | acactgggtg tgaaccagcg cggcgagctg tgcgtccgtg gccccatgat catgagcggc |
| 1201 | tacgttaaca accccgaggc tacaaacgct ctcatcgaca aggacggctg gctgcacagc |
| 1261 | ggcgacatcg cctactggga cgaggacgag cacttcttca tcgtggaccg gctgaagagc |
| 1321 | ctgatcaaat acaagggcta ccaggtgagt ctatgggacg cttgatgttt tctttcccct |
| 1381 | tcttttctat ggttaagttc atgtcatagg aaggggataa gtaacagggt acagtttaga |
| 1441 | atgggaaaca gacgaatgat tgcatcagtg tggaagtctc aggatcgttt tagtttcttt |
| 1501 | tatttgctgt tcataacaat tgttttcttt tgtttaattc ttgctttctt tttttttctt |
| 1561 | ctccgcaatt tttactatta tacttaatgc cttaacattg tgtataacaa aaggaaatat |
| 1621 | ctctgagata cattaagtaa cttaaaaaaa aactttacac agtctgccta gtacattact |
| 1681 | atttggaata tatgtgtgct tatttgcata ttcataatct ccctacttta ttttctttta |
| 1741 | tttttaattg atacataatc attatacata tttatgggtt aaagtgtaat gttttaatat |
| 1801 | gtgtacacat attgaccaaa tcagggtaat tttgcatttg taattttaaa aaatgctttc |
| 1861 | ttcttttaat atactttttt gtttatctta tttctaatac tttccctaat ctctttcttt |
| 1921 | cagggcaata atgatacaat gtatcatgcc tctttgcacc attctaaaga ataacagtga |
| 1981 | taatttctgg gttaaggtaa gtgcaatatc tctgcatata aatatttctg catataaatt |
| 2041 | gtaactgatg taagaggttt catattgcta atagcagcta caatccagct accattctgc |
| 2101 | ttttatttta tggttgggat aaggctggat tattctgagt ccaagctagg cccttttgct |
| 2161 | aatcatgttc atacctctta tcttcctccc acaggtagcc ccagccgaac tggagagcat |
| 2221 | cctgctgcaa caccccaaca tcttcgacgc cggggtcgcc ggcctgcccg acgacgatgc |
| 2281 | cggcgagctg cccgccgcag tcgtcgtgct ggaacacggt aaaaccatga ccgagaagga |
| 2341 | gatcgtggac tatgtggcca gccaggttac aaccgccaag aagctgcgcg gtggtgttgt |
| 2401 | gttcgtggac gaggtgccta aaggactgac cggcaagttg gacgcccgca agatccgcga |
| 2461 | gattctcatt aaggccaaga agggggcaa gatcgccgtg taa |

### Example 10: Cre-Mediated loxP Removal in LSL.mKate2.ssLuc2.HBB ROSA26 Knock-In Mice Promotes Robust mKate2 Expression

A knock-in (ki) mouse model was generated harboring the LSL.mKate2.ssLuc2.HBB transgene at the ROSA26 safe harbor locus (FIG. 10A) to assess ASO-mediated splice-switching activity in vivo. Sequence-verified, mouse embryonic stem cell (mESC) clones expressing the reporter vector were microinjected into C57BL/6J 8-cell blastocysts homozygous for the albino allele (TyrC/TyrC). Viable embryos were then transferred into the uteri of pseudopregnant C57BL/6J albino (TyrC/TyrC) females the following day. The resulting chimeric animals were scored by coat color and males with a high percentage of black pigmented fur were selected to breed with albino C57BL/6J female mice. The production of G1 black offspring indicated possible germline transmission, which was confirmed by genotyping. Sequence-verified, single-insertion G1 heterozygous animals that originated from a single microinjected mESC clone were transferred to a production colony (LSL.mKate2.ssLuc2.HBB) for further characterization.

To verify whether Cre-mediated recombination could induce transgene expression, the LSL.mKate2.ssLuc2.HBB strain was crossed with ROSA26.Cre.ki (Cre.ki) mice (FIG. 10B). The resulting F1 heterozygous mice were then assessed for reporter expression using mKate2 as a readout. As mKate2 should be constitutively expressed under a CAG promoter after loxP removal, mKate2 fluorescence was first quantified in the body of live mice revealing that mKate2.ssLuc2.HBB animals specifically expressed mKate2 as compared to animals from the parental colonies (FIG. 10C-10D). Furthermore, while ex vivo tissue weight showed no significant change across the three strains (FIG. 10E), mKate2 tissue fluorescence was measurably increased in the brain, kidney, liver, heart, and skin (ear) of mKate2.ssLuc2.HBB animals (FIG. 10F-10G). A significant increase in mKate2 expression was not observed in the spleen, likely due to imaging limitations for small whole tissue samples from young animals (<1 month of age). Together, these data show that mKate2 is widely expressed following loxp removal and thus the mKate2.ssLuc2.HBB strain can be used to evaluate ASO activity in live mice. Additionally, a significant increase in bioluminescence was observed in mKate2.ssLuc2.HBB animals when measured from the tail, front paw and back paw (FIG. 10H).

### Example 11: ssASO1 Intravenous (IV) Injection Promotes Robust, Dose-Dependent Splice-Switching in mKate2.ssLuc2.HBB Mice

In vivo ASO activity and biodistribution analyses currently rely upon time- and cost-intensive biochemical assays, which limit throughput and require large cohorts of animals. Thus, the mKate2.ssLuc2.HBB ki mouse model was utilized to quantify ASO activity over the course of 6 weeks in live animals. Given that intravenous (IV) administration is commonly used to deliver a wide range of therapeutics, ASO-mediated Luc2 splice-switching dynamics were evaluated in mixed gender ki/ki mice at 2-6 months of age following a single IV injection of saline or ssASO1 (25, 100, or 250 μg final dose) (FIG. 11A). All animals responded well with no acute toxicity or weight loss noted over the course of the experiment, suggesting the dosing was well-tolerated (FIG. 11B). Live animal bioluminescence imaging was then used to assess how ASO dosing impacts Luc2 pre-mRNA splicing over time in unique tissues (FIG. 11C). Specifically, isoflurane anesthetized animals were imaged for bioluminescence and the signal emanating from the abdomen, head, and both eyes were quantified 10 minutes after intraperitoneal (IP) injection of 5 mg D-luciferin (FIGS. 11C-11F). ASOs are known to be largely cleared through the liver post administration. Accordingly, the highest bioluminescence signal was observed in the abdomen post dosing, which was driven by Luc2 signal originating from the liver (FIG. 11D). Despite the highest level of bioluminescence being detected in the abdomen, a steady increase in Luc2 splice-switching was observed for each body part analyzed over the course of the first 7 days post dosing (FIGS. 11D-11F). The activity remained largely stable for the next 5 weeks, with some decay observed in the head and eyes near the end of the study. These data reveal that ASO-mediated splice-switching increases quickly post IV dosing and is then maintained for long periods of time in multiple tissues. A dose-dependent increase in Luc2 splice-switching was also observed in the abdomen, head, and eyes (FIGS. 11D-11F). These data show that ASO activity is not only stable over time, but also dose-dependent following IV injection. Surprisingly, ASO-mediated Luc2 splice-switching was observed in the head even though ASOs do not readily permeate the blood-brain barrier (BBB) following peripheral administration. To test whether this signal may result from ASO uptake in blood vessel endothelium, bioluminescence was quantified in the tail for comparison (FIGS. 11G-11I). A time- and dose-dependent increase in bioluminescence was observed in the tail of ssASO1-treated mice suggesting that the activity in the head could be at least partially attributed to ASO uptake in the blood vessels and/or skin post IV administration. Together, these data reveal that the mKate2.ssLuc2.HBB mouse model sensitively reads out ASO splice-switching in vivo across multiple tissues in a dose- and time-dependent manner.

### Example 12: ssASO1 Intracerebroventricular (ICV) Injection Leads to Sustained Luc2 Signal in the Brain and Clearance Through the Liver

ASO-mediated Luc2 splice-switching was next evaluated in mKate2.ssLuc2.HBB ki/ki mixed gender mice at 2-6 months of age following a single intracerebroventricular (ICV) administration of saline or ssASO1 (12.5, 50, or 100 μg final dose) (FIG. 12A). Mice dosed with 100 μg ASO showed minor acute side effects including lethargy immediately following injection (FIG. 12B). However, the mice recovered well post surgery with normal activity resumed within 24 hours. Additionally, no weight loss was observed over the course of the experiment (FIG. 12C). These data align with previous results showing ICV injection of ssASO1 triggers dose-dependent acute side effects, but does not induce long-term brain gliosis. Luc2 signal was next quantified in the abdomen, head, and eyes to assess ASO splice-switching dynamics using bioluminescence as a readout (FIGS. 12D-12G). A strong dose-dependent induction of bioluminescence was observed in the abdomen of ssASO1-treated animals as compared to saline-injected mice (FIG. 12E). These data are consistent with Luc2 activity measurements following IV injection, suggesting ASOs are likely cleared using similar mechanisms despite of the route of administration used. However, the dynamics of clearance are unique as bioluminescence signal increased linearly for 7 or 3 days following IV or ICV injection, respectively, before plateauing for the remainder of the study (FIG. 11D vs FIG. 12E). In addition to the abdomen, robust dose- and time-dependent Luc2 splice-switching activity was also noted in the brain of ssASO1 ICV-injected animals (FIG. 12F). These data are consistent with previous reports showing that ASO ICV administration leads to long-term ASO activity in the brain. However, unlike previous studies which are limited in resolution due to requiring end-of-life observations, the live animal imaging studies conducted herein reveal that ASO distribution changes dramatically in the days following injection (see FIG. 12D). The bioluminescent signal is strongest near the injection site, the right lateral ventricle, the day immediately post dosing. Over the course of the week, however, the signal can be observed distributing to the left lateral ventricle and then slowly throughout larger sections of the brain. These data reveal that ASOs are distributed dynamically throughout the brain and become differentially active in unique brain regions following ICV injection. Bioluminescence signal in the eyes following ICV injection was also observed, suggesting that ASOs are active and distribute to additional tissues following injection in the brain (FIG. 12G). Together, these results show that ASO dynamics can be observed at high resolution over time in multiple tissues using the mKate2.ssLuc2.HBB ki mouse.

### Example 13: Luc2 Splice-Switching is Robust in the Eye after Intravitreal (IVT) Injection of ssASO1

The use of ASO therapies for the treatment of ocular disorders has become more prevalent in recent years. However, data exploring the longevity of ASO activity and clearance from the eye are lacking. Thus, Luc2 splice-switching was next assessed in mKate2.ssLuc2.HBB ki/ki mixed gender mice at 2-6 months of age after a single intravitreal (IVT) injection of saline or ssASO1 (6.25, 25, or 50 μg/eye final dose) (FIG. 13A). All mice responded well to treatment with no acute toxicity noted the day following injection and steady weight gain observed over the course of the experiment (FIG. 13B). Luc2 splice-switching was then observed in the abdomen, head, and eyes using live, whole animal bioluminescence imaging (FIG. 13C). Consistent to the splice-switching response measured after IV and ICV administration, a strong dose-dependent increase in bioluminescence was observed in the abdomen post injection, which was largely localized to the liver (FIG. 13D). Signal increased steadily for 7 days post injection in all ssASO1-dosed animals and then plateaued until the end of the study where a slight decrease in signal was detected. A strong increase in Luc2 splice-switching activity was also observed in the head of ssASO1-treated mice as compared to saline control animals (FIG. 13E). However, unlike the robust, long-lasting activity observed for all other tissues, signal in the head steadily increased the first week following IVT injection then quickly diminished thereafter until little difference could be detected between treatment groups 6 weeks following ASO administration. These data suggest that ASO accumulates in the brain following IVT injection, but long-term activity is lacking. In contrast, a robust dose-dependent increase in Luc2 bioluminescence was measured in the eye over the course of the study (FIG. 13F). Together, these data suggest that ASO-mediated splice-switching dynamics differs in unique tissues following IVT injection.

### Example 14: Materials and Methods

### Splice-Switching EGFP Construct Design

Enhanced green fluorescent protein (EGFP) was split at nucleotide (nt) 471 with a large exon containing β-globin (HBB, nts 472-1321) carrying a C to T mutation at nt 1125. The HBB exon sequence was further mutated at nts 1128-1129 from TA to GT to decrease non-ASO mediated background EGFP expression. The EGFP471.HBBta 1128-1129gt (ssEGFP-HBB) construct was synthesized using the wild-type HBB sequence (General Biosystems Inc.) and point mutations were added using PCR mutagenesis, and was cloned into a pcDNA3.1 (+) expression vector. All nt sites are counted from the EGFP ATG start codon and the full ssEGFP.HBB sequence is provided in Table 4.

### Splice-Switching Luc2.HBB Cassette Construct Design

Firefly luciferase (Luc2) was split at nucleotide (nt) 1344 with an exon containing β-globin (HBB, nts 1345-2194) carrying a C to T mutation at nt 1998. The HBB exon sequence was further mutated at nts 2001-2002 from TA to GT to decrease non ASO-mediated background Luc2 expression. All nt sites are counted from the Luc2 ATG start codon and the full ssLuc2.HBB cassette sequence is provided in Table 6.

### Oligonucleotides

All ASOs were synthesized following standard phosphoramidite protocols as described previously.25 ASOs used in this study were the following (ID: base sequence, sugar sequence): ssNC1 (SEQ ID NO: 17): GCAAATTCETATTCEC, LDLDLDLDLDLDLDLD; ssASO1 (SEQ ID NO: 18): AETTACCTTAAEECAG, LLDDLDDLLDDLLDLL; ssASO2 (SEQ ID NO: 19): GAGATATTGEACTTAEET, LDLLDLLDLLDDLDDLLL. ssASO3 (SEQ ID NO: 20): GAGATATTGEACTTAEET, LDLLDLLDLLDDLDDMLL. E corresponds to 5-methyl C in the base sequences, and in the sugar sequence M is 2′-O-methoxyethyl ribose, L is LNA, and D is DNA. All backbones are phosphorothioate.

Formulation of ssASO1-Lipid Nanoparticles (LNPs)

A microfluidic approach was used to prepare the ssASO1-LNP formulation as described previously. Briefly, dilinoleylmethyl-4-dimethylaminobutyrate (MC3, MedChemExpress), 1,2-distearoyl-sn-glycero-3-phosphocholine (DSPC, Avanti Polar Lipids), cholesterol (Sigma), and 1,2-dimyristoyl-rac-glycero-3-[methoxypolyethylene glycol-2000] (DMG-PEG2000) (Avanti Polar Lipids) were dissolved in ethanol at a ratio of (40:10:48:2.0). ssASO1 was dissolved in citrate buffer (25 mM, pH 4.0). The lipids and ASO were mixed at a 1:3 ratio and a flow rate of 12 ml/min using microfluidic laminar mixing (NanoAssemblr™ Benchtop, Precision NanoSystems). ssASO1-LNPs were purified from free components using centrifugal ultrafiltration (MWCO 10 kD, Amicon, Sigma) and buffer exchanged into RNase-free PBS. The purified formulation was analyzed for mean particle diameter and size distribution (percent polydispersity, % PD) using a DynaPro plate reader III (Wyatt Technology). ASO encapsulation and concentration was measured using hydrophilic interaction liquid chromatography.

### HEK293 Cell Culture and Stable Cell Line Generation Using Fluorescence Activated Cell Sorting (FACS)

HEK293 cells stably expressing a nuclear localized CRE recombinase (SC004-Puro, GenTarget Inc.) were cultured in DMEM high glucose supplemented with 10% fetal bovine serum (FBS, VWR), 1% penicillin-streptomycin (P-S, Thermo), 2 mM L-glutamine (Invitrogen), and non-essential amino acids (Invitrogen). 1 day prior to transfection, cells were plated at a density of 2×106 on 100 mm poly-L-lysine (PLL)-coated (Sigma) dishes. Cells were transfected in culture media lacking P-S with Super PiggyBac Transposase (PB210PA-1, System Biosciences) and EGFP reporter vector (1:2.5) using lipofectamine 2000 (Invitrogen) according to the manufacturer's instructions. The EGFP vector was synthesized with flanking piggyBac inverted terminal repeats (ITRs) and cloned into a pBR322 vector, to generate a 12,692 base-pair dsDNA construct (Genscript). Full sequence of the construct is available upon request. Medium was exchanged 24 hrs post transfection. Cells were cultured and split regularly when confluent. Every 1-2 weeks FACS was used to isolate mKate2 positive cells until a pooled stable cell line was obtained. Cell sorting was performed on a BD FACSAria™ Fusion equipped with 5 lasers (355, 405, 488, 561, and 638 nm). The instrument was set up with a nozzle size of 100 μm at a frequency of 32 kHz and pressure of 20 psi.

### HEK293 Cell Culture Treatment and Fixation

Stable HEK293 cells were plated at a density of 1.25×10⁵ cells/ml in 35 mm no. 1.5 coverslip dishes coated with PDL (P35GC-1.5-14-C, MatTek Corporation). At ~50% confluency cells were transfected with ASO using lipofectamine 2000 (Invitrogen) according to the manufacturer's instructions. After 2 days, cells were washed with PBS and fixed using 3% paraformaldehyde (PFA, Electron Microscopy Sciences) for 20 minutes (min) at room temperature (rt). Fixed cells were washed with PBS and stored at 4° C. until imaging was performed.

### Dissociated Primary Murine Cortical Neuron Cultures

Mouse embryonic cortical neurons were cultured as described previously. Briefly, cortices from day 15 C57BL/6J transgenic embryos (E15) were dissected, washed with HBSS (Invitrogen), and incubated at 37° C. in HBSS supplemented with 0.25% trypsin (Invitrogen) and DNase I (Roche CustomBiotech). Tissue was washed with HBSS and triturated in plating media containing DNase I (Gibco Neurobasal Medium (Thermo), 20% heat-inactivated horse serum (Thermo), 25 mM sucrose, and 0.25% Gibco GlutaMAX (Thermo)). Dissociated cells were centrifuged at 125 μg for 5 min at 4° C., resuspended in a plating medium, and plated on PLL-coated (Sigma) plates. The medium was replaced with NbActiv4 (BrainBits) after 24 hrs and was renewed using 50% exchange every 3-4 days.

### Dissociated Primary Murine Mixed Glial Cultures (Microglia and Astrocytes)

Primary mouse glial cells were cultured from day 3 C57BL/6J pups (P3) as described previously. Briefly, cortices were dissected, washed with cold HBSS (Invitrogen), and incubated in 0.25% trypsin (Invitrogen). Cortices were washed in glial culture medium (DMEM high glucose supplemented with 10% FBS and 1% P-S (Thermo)) and triturated. Dissociated cells were filtered and the cell suspension was centrifuged at 700 μg for 5 min. The cell pellet was resuspended in glial culture medium and plated in PLL-coated (Sigma) 225 cm² flasks at a density of 8 cortices/flask. After 4 days, the attached cells were washed with PBS and fresh glial culture medium was added. Primary microglia and astrocytes were differentially separated after an additional 7 days in culture.

Mixed glial cultures were agitated at 200 rpm for 2 hrs at 37° C. with 5% CO2. The detached microglia were centrifuged at 700 μg for 5 min, resuspended in glial culture medium supplemented with 10 ng/mL mCSF1 (416-ML-050, R&D Biosystems), and plated on PLL-coated (Sigma) plates. The flasks containing an intact astrocyte layer were washed with PBS, glial culture medium was added, and were agitated at 260 rpm for 24 hrs. The medium was removed, flasks were washed with PBS, and 0.25% trypsin (Thermo) diluted 1:5 in DMEM/F12 was added. The detached astrocyte layer was collected, excess trypsin was removed, and fresh 0.25% trypsin (Thermo) was added. Occasional manual agitation was used to promote full cell detachment. Upon obtaining a single cell suspension, cells were centrifuged at 700 μg for 5 min. Astrocytes were resuspended in glial cell culture medium and plated in PLL-coated (Sigma) plates.

### Transgenic CNS Cell Culture Treatments and Fixation

Primary astrocytes were plated at a density of 1×10⁵ cells/ml and cortical neurons as well as microglia were plated at a density of 4×10⁵ cells/ml for all studies. For concentration- and time-dependent EGFP splice-switching analyses cells were plated in either black 96-well CellCarrier Ultra (6055302, Perkin Elmer) or 24-well no. 1.5 coverslip (MatTek Corporation) plates coated with PLL (Sigma), respectively. At DIV3 (astrocytes/neurons) or DIVO (microglia), cells were treated with ASO (0, 0.5, 1, 2.5, 10, and 20 μM), ssASO1-LNPs (0, 1, 10, 20, 100, and 200 nM), or 10 μM ssASO1 (time course study). After 7 days or 0, 1, 4, and 7 days (time course study), cells were washed with PBS and fixed using 3% PFA (Electron Microscopy Sciences) for 20 min at rt. Fixed cells were washed with PBS and stored at 4° C. until imaging was performed.

### Transgenic Mouse Generation and Animals

In order to facilitate homologous recombination, the transgenic reporter sequence was synthesized with flanking ROSA26 left and right homology arms and cloned into a pUC57-Brick vector, generating a 13,012 base-pair dsDNA plasmid donor (GenScript). Full sequence of the construct is available upon request. We then employed the CRISPR/Cas9 system using the following ROSA26 guide RNA sequence to improve knock-in (ki) efficiency: ACTCCAGTCTTTCTAGAAGATGG (SEQ ID NO.: 2). This sgRNA (39 ng/μl, Synthego) and Alt-R S.p Cas9 Nuclease V3 (50 ng/μl, IDT #1081059) were mixed and incubated at rt for 10 min. A microinjection mixture containing the pre-formed RNP and 10 ng/μl dsDNA plasmid donor was then formed in TE buffer pH 7.6 (T0230, Teknova) and filtered through Spin-X columns (8160, Costar) by centrifugation at 16,000 μg for 5 min. The filtered mixture was microinjected into both pronuclei of E1.5 two-cell-stage C57BL/6J embryos, which were then transferred into the oviducts of pseudo pregnant females.

Genomic DNA was isolated from tail biopsies of G0 mosaic founders and used for both loss-of-allele (LOA) assays and full-length sequence verification. First, G0 founders were pre-screened using LOA assays to identify targeted ki animals having less than two copies of the native ROSA26 insertion site and gaining >0.1 copies of the ki donor. LOA assays were performed using a QIAcuity 8 Digital PCR system (911052, Qiagen) using the following primers and probes (IDT) (ID: sequence): CAG_fwd: CTTCCCTCGTGATCTGCAA (SEQ ID NO.: 3); CAG_probe: TTTCTAGGTAACCGATATCCCTGC (SEQ ID NO.: 4); CAG_rev: GCTATGAACTAATGACCCCGT (SEQ ID NO.: 5); BGHpA_fwd: GGGGCTCGATCCTCTAGTTG (SEQ ID NO.: 6); BGHpA_probe: CGCGCCGGCTAGAAGATGG (SEQ ID NO.: 7); BGHpA_rev: ACCTGTTCAATTCCCCTGCA (SEQ ID NO.: 8). LOA-positive animals were then verified by sequencing the full-length insert. Nested long-range PCR was used to generate template for amplicon sequencing. As the insert exceeded 10 kb, we amplified the full-length sequence in two halves—5′ and 3′ using the following primers (IDT) (ID: sequence): 5′_fwd1: GCCTTGGGTCATGTCCTACC (SEQ ID NO.: 9); 3′_rev1: TAGGGAGGTCGCAGTATCTG (SEQ ID NO.: 10): 5′_fwd2: AACAAGTGCTCCATGCTGGA (SEQ ID NO.: 11); 3′_rev2: CTTTAGAATGGTGCAAAGAGGC (SEQ ID NO.: 12); 3′_fwd1: ACCCGCTAAGAACCTCAAGATG (SEQ ID NO.: 13); 3′_rev1: TGGGAATTGAACTCAGGACTTC (SEQ ID NO.: 14); 3′_fwd2: GCCTCATCTACAACGTCAAGAT (SEQ ID NO.: 15); 3′_rev2: GTGCTCAGCAAGTCCTAGGG (SEQ ID NO.: 16). Barcoded sequence libraries were prepared from the amplicons using library kits (SQK-LSK109, EXP-NBD104, EXP-NBD114, Oxford Nanopore Technologies) and sequenced on a GridION Mk1 sequencer (Oxford Nanopore Technologies) according to the manufacturer's instructions. The sequence data was mapped to the donor reference using minimap2 and alignments were visualized/analyzed using Integrative Genomics Viewer. Sequence verified G0 animals at breeding age (>6 weeks) were crossed with C57BL/6J animals to produce G1 animals. G1 animals were genotyped as described above for G0 animals, with the following exception: G1 animals were pre-screened using LOA assays to identify targeting ki animals having 1 copy of the native Rosa26 insertion site and 1 copy of the ki donor. Single ROSA26 insertion animals were then transferred into a production colony.

Embryos and pups used for in vitro primary CNS cell cultures resulted from breeding ki/wt females with ki/ki males. Male ki/ki mice between the ages of 3-6 months were used for the in vivo, ICV dosing experiments. Mice were housed on a regular light/dark cycle (14:10 hrs) with ad libitum access to food (LabDiet 5010) and water. All injections and tissue collections were conducted during the light phase. All animal care and handling procedures were reviewed and approved by the Genentech Institutional Animal Care and Use Committee and were conducted in full compliance with regulatory statutes, Institutional Animal Care and Use Committee policies, and National Institutes of Health guidelines.

LSL.mKate2.ssLuc2.HBB Reporter Construct Design

To target the ROSA26 locus by homologous recombination, ROSA26 left and right homology arm sequences were added to the ends of a 7,960 bp DNA sequence containing the following elements: a CAG promoter, a loxP-Stop-loxP (LSL) cassette, a mKate2 reporter, the split luciferase reporter cassette (ssLuc2.HBB), and a BGH polyA termination signal. The complete 13,691 bp insert was synthesized and cloned into a pBlightTK vector to generate the final 17,973 bp DNA plasmid donor (GenScript).

Mouse Embryonic Stem Cell (mESC) Culture and Electroporation

C57BL/6J mouse embryonic stem cells (mESCs; Genentech, Inc.) were cultured at 37° C. and 5% CO2 on 10 cm dishes (430239, Corning) coated with a monolayer of mouse embryonic fibroblasts (MEFs; PMEF-NX-C, Millipore-Sigma). The mESC media (1× KnockOut DMEM (10829018, Gibco), 15% v/v FBS (SH30070.03, Cytiva), 2 mM L-glutamine (25030081, Gibco), 1×MEM non-essential amino acids (11140050, Gibco), 1,000 units/mL of mouse LIF protein (ESG1107, Millipore-Sigma), and 0.0007% v/v 2-mercaptoethanol) was changed daily until 80-90% confluency was reached.

Prior to electroporation, the circular LSL.mKate2.ssLuc2.HBB donor DNA (100 μg) was linearized with 200 U of KpnI-HF (NEB #R3142S) in 1× rCutSmart buffer at 37° C. overnight. The DNA was precipitated and the final concentration was determined using a NanoDrop 2000. On the day of electroporation, the cells were dissociated using 0.25% trypsin-EDTA (25200056, Gibco), centrifuged at 1000 μg for 5 min, and the resulting cell pellet was resuspended in DPBS. Cells (20×106) were mixed gently with donor DNA (10 μg), transferred to an electroporation cuvette (1652088, Bio-Rad), and electroporated with a square waveform at 240V and 550 uF using a Bio-Rad Gene Pulser Xcell electroporator (1652661, Bio-Rad). The transfected cells were then transferred to a 10 cm dish coated with MEFs. The media was changed daily with mESC media supplemented with 2 μM ganciclovir (G2536, Sigma-Aldrich) and 0.2 mg/mL Geneticin (10131027, Gibco) to select for transfected cells. 7-9 days following transfection, individual colonies were harvested by aspiration and dispensed into 96-well plates (353072, Corning) containing 0.25% trypsin-EDTA to dissociate the colonies. Dissociated cells were transferred into 96-well plates containing mESC media and coated with MEFs. Cultures were split into two fresh 96-well plates after two days: one for preservation in cold cryopreservation media (sterile filtered FBS with 10% v/v DMSO) and the second for genotype pre-screening, as described below.

mESC Microinjection and LSL.mKate2.ssLuc2.HBB Mouse Colony Generation

Sequence-verified mESC clones were thawed and expanded until 50-60% confluency was reached. Cells were dissociated in 0.25% trypsin-EDTA, resuspended in mESC media without LIF, and centrifuged at 2,000 μg for 5 min. The resulting cell pellet was resuspended in mESC media without LIF and C57BL/6J 8-cell blastocysts homozygous for the albino allele (TyrC/TyrC) were microinjected with 7-9 cells/blastocyst. Injected embryos were cultured in mESC media without LIF overnight in a 37° C. incubator with 5% CO2. Viable embryos were then transferred into the uteri of pseudopregnant C57BL/6J albino (TyrC/TyrC) females. Approximately 19 days post-implantation, chimeras were born and scored by coat color. Chimeric males with a high percentage of their fur pigmented with black patches on white background were selected to breed with albino C57BL/6J female mice. The production of G1 black offspring was an indicator of germline transmission. Genomic DNA was then isolated from tail biopsies of all black offspring and genotyped using the same process as described above for the mESCs. Sequence-verified G1 heterozygous animals that originated from a single microinjected mESC clone were then transferred to a production colony (LSL.mKate2.ssLuc2.HBB) for further characterization.

### Intracerebroventricular (ICV) Injections

A freehand ICV surgical injection method was used. Animals were anesthetized with isoflurane, the skull was shaved, and the skin was cleaned with either antimicrobial betadine solution and 70% ethanol, or Chloraprep (260100, BD Biosciences). The bregma was identified using a midline skin incision between the occiput and forehead. The needle of a 26-gauge 25 μl Hamilton syringe was fitted with polyethylene tubing cut so that no more than 3 mm of the needle was available to penetrate the brain. At coordinates of 1-2 mm to the right of the midline and posterior −0.5 mm from bregma, the needle was pushed through the skull to a depth of −2.8 mm and one min was given for the brain to seal. Next, a bolus injection of 5 μl ASO or saline was injected at a rate of 1 μl/second (s). One min after the injection, the needle was withdrawn and the tissue was closed using tissue glue. To reduce any pain from the procedure, buprenorphine was administered for up to 3 days.

### Mouse Observations Post ICV Dosing

Acute side effects monitored included reduced movement, ataxia, and hunched body. These 3 observations were scored as follows: 0-normal, 1-mild, 2-moderate, 3-severe. An even more severe score of 4 was given if seizures, death, or catatonic behavior was observed with no signs of improvement after 2 hrs. Acute side effect scoring was performed at 30 min and 1 hr post ICV injection, with the average score reported. Individual body weights were measured pre-ICV injection and at 1 as well as 2 weeks post-ICV injection.

### Intravenous (IV) Injections

A freehand tail vein IV injection method was used. Mixed gender animals between 1 and 3 months of age were heated for 4-5 min with an overhead red-bulb lamp to promote vasodilation and were continuously monitored for signs of overheating. Mice were then restrained without anesthesia using an adequately sized restrainer for dose administration and the tail was swabbed with a 70% alcohol pad. The lateral tail vein was identified and the needle of a 29-gauge×½ inch, 0.5 mL syringe (26028, EXEL INT) was inserted parallel to the vein at a depth of 2-4 mm into the lumen. Saline and/or pooled ASO-LNPs were then injected slowly at a maximum dose volume of 8 mL/kg. After administration, the needle was slowly removed and the injection site was held off with gauze until hemostasis was achieved. All animals were monitored for 1 h following injection for potential adverse effects.

### Intravitreal (IVT) Injections

IVT injection was performed on both eyes of mixed gender 2-6 month old animals between 1-3 months of age. Mice were anesthetized with 75-80 mg/kg ketamine, 15 mg/kg xylazine IP in 150-300 μl sterile saline. Depth of anesthesia was confirmed by toe pinch/absence of reflexes. Eyes were dilated with 1% tropicamide drops (Akorn Inc, Lake Forest, IL, USA), and cleaned with BETADINE 5% Sterile Ophthalmic Prep Solution (Alcon, Fort Worth, TX, USA) 5 min before injection. Under a surgical microscope, an entrance site was then created using a STERiJECT 33G ½ needle (TSK, Japan) at the posterior limbus. A Hamilton syringe with a 34 gauge blunt needle (Hamilton Company, Reno, NV, USA) was used to deliver 1.2 μl of ASO or saline through the existing entrance site. Neomycin and polymyxin B sulfate/bacitracin zinc ophthalmic ointment (Bausch and Lomb, Bridgewater, NJ, USA) was then applied. A single dose of 3.25 mg/kg Ethiqa XR (Fidelis Animal Health, North Brunswick, NJ, USA) was also administered to provide analgesia for the next 3 days. After surgery, mice were given 0.7 mg/kg Antisedan (Akorn Inc, Lake Forest, IL, USA) to promote anesthesia recovery. All animals were allowed to recover on heating pad and were monitored until fully awake/ambulatory before returning to the housing room. Care was taken to avoid the lens during injection and if the lens was damaged, the mice were excluded from subsequent analysis.

### Tissue Collection

All mice were anesthetized with 2.5% tribromoethanol (0.5 ml/25 gram body weight) and transcardially perfused with PBS followed immediately by 4% PFA. Tissues were removed and imaged as described below. Prior to histology, tissues were post-fixed overnight in PFA and stored in PBS at 4° C. until sectioning was performed.

### In Vivo and Ex Vivo Tissue Fluorescence Imaging

For in vivo mKate2 and EGFP fluorescence expression analysis, animals were anesthetized with isoflurane prior to and during imaging. All fluorescence intensity measurements were acquired using a Lago optical imaging system (Spectral Instruments Imaging), which can image up to 10 animals simultaneously. For all analyses the following settings were used for image acquisition: mKate2 (ex. 570, em. 650), EGFP (ex. 465, em. 510), 20 s exposure at 15% power, and binning was set to 2 with a 25 or 20 cm field of view for in vivo and ex vivo imaging, respectively. All fluorescence images were analyzed using Aura Software (Spectral Instruments Imaging).

### Tissue Sectioning, Immunofluorescence (IF) Staining, Imaging, and Analysis

Following post-fixation in 4% PFA overnight at 4° C., mouse tissues were cryoprotected in 30% sucrose at 4° C. for at least 24 hrs. Using the Leica CM3050S cryostat, 30-μm-thick serial, coronal tissue sections were then obtained and stored in a PBS solution containing 0.2% sodium azide (Sigma) at 4° C.

Prior to immunofluorescent staining, sections were washed with PBS. For Olig2 detection, antigen retrieval was performed by incubating tissue sections in Target Retrieval Solution, pH 9.0 (Dako) at 85° C. for 6 min. All tissue sections were then permeabilized with 0.1% Triton X-100 (Sigma-Aldrich). Nonspecific antibodies were blocked with 20% normal goat (Sigma Aldrich) at rt for 1 hr. Tissue sections were then incubated in primary antibody overnight at 4° C. The following primary antibodies were used for this study: rabbit anti-ASO (1:2000, JW138-442, Genentech, Inc.), chicken anti-GFP (1:1000 or 1:2000, abcam), rabbit anti-Iba1 (1:500, Dako), guinea pig anti-NeuN (1:500, Synaptic Systems), rabbit anti-S100B (1:500, Proteintech), and rabbit anti-Olig2 (1:300, Sigma). Following washes in PBS, tissue sections were then incubated in secondary antibodies at rt for 1 hr. The following secondary antibodies (Invitrogen) were used for this study all at a concentration of 1:1000: goat anti-chicken AF488, goat anti-guinea pig AF647, goat anti-rabbit AF647, and goat anti-rat AF647. Tissue sections were cover slipped with Fluoromount-G mounting medium with DAPI (Thermo).

Immunofluorescent images for ASO and EGFP were acquired using an Olympus VS200 slide scanner and processed using OlyVIA 2.4 (Olympus) and Fiji (ImageJ) software. Fluorescence intensities were analyzed by first applying a top hat transformation to each channel in order to remove the influence of diffuse background autofluorescence as well as bright artifacts. Then, an empirically set intensity threshold was applied to identify positively stained pixels in each channel. At least eight serial sections 360-μm apart were quantified for both ASO and EGFP fluorescence intensity, normalized to the total DAPI+ area per section, and averages per animal were plotted. To quantify the co-labelling of EGFP-positive cells with various CNS cell types, 9 z-stacks images (1-μm intervals) were acquired using a Zeiss Apotome 2 and processed using ZEN (Zeiss) and Fiji software. Two sections from each sample were measured for Mander's coefficient, which describes the amount of overlap between EGFP and its respective counterstain of either S100B, Iba1, NeuN, or Olig2. Quantifications for both were performed by an individual blind to sample identity using the public domain NIH Image analysis software, ImageJ.

### Animals

All animal care/handling procedures were reviewed and approved by the Genentech Institutional Animal Care and Use Committee and were conducted in full compliance with regulatory statutes, Institutional Animal Care and Use Committee policies, and National Institutes of Health guidelines. Mice were housed on a regular light/dark cycle (14:10 hrs) with ad libitum access to food (LabDiet 5010) and water.

### Statistical Analysis

Data plotting and statistical analyses were performed using Prism 9.2.0 (GraphPad Software). Results are presented as mean±SEM and all experiments were completed in triplicate, unless otherwise noted. Data involving multiple comparisons were analyzed using one- or two-way ANOVA coupled with multiple comparisons Dunnett's or Tukey's tests, as described in the figure legends. Unless otherwise noted, statistical significance was defined as p<0.05.

1. A method of assessing delivery and/or splice-switching activity of an antisense oligonucleotide (ASO) comprising
(i) introducing the ASO to a transgenic mouse, wherein the transgenic mouse comprises a plurality of cells which comprise a genetic sequence that is transcribed as a pre-mRNA, said pre-mRNA comprising a first segment encoding a first portion of a reporter protein, a cryptic sequence, and a second segment encoding a second portion of the reporter protein,
wherein the ASO alters splicing of the pre-mRNA to exclude the cryptic sequence from a mature mRNA transcript, thereby causing expression of a functional form of the reporter protein,
wherein the cryptic sequence comprises a sequence comprising at least 95% sequence identity to the nucleotide sequence set forth in SEQ ID NO.: 1 comprising nucleotide substitutions of T657G and A658T which result in decreased exclusion of the cryptic sequence in the absence of the ASO when compared to exclusion of the cryptic gene without the nucleotide substitutions of T657G and A658T;
and
(ii) detecting the expression of the reporter protein to assess the delivery and/or splice-switching activity of the ASO in the plurality of cells.

2. The method of claim 1, wherein an amount of ASO that is introduced to the transgenic mouse is about 50 μg or more.

3. The method of claim 1, wherein a concentration of ASO that is introduced to the transgenic mouse is about 0.5 μM or more.

4. The method of claim 1, wherein the sequence that is transcribed as a pre-mRNA is expressed ubiquitously in the transgenic mouse or is expressed in specific tissues or cells.

5.-7. (canceled)

8. The method of claim 1, wherein the cryptic sequence comprises the nucleotide sequence set forth in SEQ ID NO.: 1.

9. A method of assessing delivery and/or splice-switching activity of an antisense oligonucleotide (ASO) comprising:
(i) introducing the ASO to a plurality of cultured cells, wherein the plurality of cultured cells are isolated from a transgenic animal comprising a genetic sequence that is transcribed as a pre-mRNA, said pre-mRNA comprising a first segment encoding a first portion of a reporter protein, a cryptic sequence, and a second segment encoding a second portion of the reporter protein,
wherein the ASO alters splicing of the pre-mRNA to exclude the cryptic sequence from a mature mRNA transcript, thereby causing expression of a functional reporter protein,
wherein the cryptic sequence comprises a sequence comprising at least 95% sequence identity to the nucleotide sequence of SEQ ID NO.: 1 comprising nucleotide substitutions of T657G and A658T which result in decreased exclusion of the cryptic sequence in the absence of the ASO when compared to exclusion of the cryptic gene without the nucleotide substitutions of T657G and A658T,
and
(ii) detecting the expression of the reporter protein to assess the delivery and/or activity of the ASO in the plurality of cultured cells.

10.-12. (canceled)

13. A method of assessing a feature of an antisense oligonucleotide (ASO) in a plurality of cells comprising:
(i) providing the ASO to the plurality of cells,
wherein the plurality of cells comprises a genetic sequence that is transcribed as a pre-mRNA, said pre-mRNA comprising a first segment encoding a first portion of a reporter protein, a cryptic sequence, and a second segment encoding a second portion of the reporter protein,
wherein the ASO alters splicing of the pre-mRNA to exclude the cryptic sequence from a mature mRNA transcript, thereby causing expression of a functional reporter protein,
wherein the cryptic sequence comprises one or more nucleotide insertions, deletions, or substitutions that result in decreased exclusion of the cryptic sequence in the absence of the ASO when compared to exclusion of the cryptic gene without said one or more nucleotide insertions, deletions, or substitutions,
and
(ii) detecting the expression of the reporter protein to assess a feature of the ASO in the plurality of cells.

14. The method of claim 13, wherein the cryptic sequence comprises a sequence of SEQ ID NO.: 1 comprising nucleotide substitutions of T657G and A658T which result in decrease of the exclusion of the cryptic sequence in the absence of the ASO.

15.-18. (canceled)

19. The method of claim 1, wherein the nucleotide substitutions of T657G and A658T decrease the exclusion of the cryptic sequence in the absence of splice-switching ASO by at least 10%, 20%, or 30%.

20. The method of claim 1, wherein the ASO comprises a nucleotide sequence that is complementary to a part of the cryptic sequence.

21. The method of claim 20, wherein the ASO is complementary to a splice donor site or a splice acceptor site that regulates splicing of the cryptic sequence.

22.-23. (canceled)

24. The method of claim 1, wherein the ASO comprises one or more chemical modifications.

25.-26. (canceled)

27. The method of claim 1, wherein the ASO is 12 to 30 nucleotides in length.

28.-31. (canceled)

32. The method of claim 1, wherein the detected expression of the reporter is indicative of presence, amount, and/or splice-switching activity of the ASO in the cell.

33. The method of claim 13, wherein the feature is a formulation, a clearance rate, a delivery route, pharmacokinetics, pharmacodynamics, uptake, tissue localization, concentration, cellular internalization, impact on gene expression, a modification, impact of genetics on ASO activity, cellular trafficking, or biodistribution.

34.-36. (canceled)

37. The method of claim 1, wherein the plurality of cells er-the-comprises a DNA cassette comprising the genetic sequence.

38.-39. (canceled)

40. The method of claim 37, wherein the DNA cassette further comprises a polynucleotide encoding a second reporter protein.

41.-43. (canceled)

44. The method of claim 37, wherein the DNA cassette comprises a promoter operably linked to the nucleic acid that is transcribed as the pre-mRNA.

45.-51. (canceled)

52. The method of claim 1, wherein the splice-switching ASO is complementary to a 5′ splice donor site of the cryptic sequence.

53. The method of claim 1, wherein the cryptic sequence is a cryptic β-globin (HBB).

54. The method of claim 53, wherein the cryptic sequence comprises the sequence set forth in SEQ ID NO: 1.

55. The method of claim 54, wherein the splice-switching ASO is complementary to SEQ ID NOs: 17-20.

56. The method of claim 1, comprising testing a plurality of splice-switching ASOs.

57. The method of claim 56, wherein each of the plurality of splice-switching ASOs comprises the same nucleotide sequence.

58. The method of claim 56, wherein each of the plurality of splice-switching ASOs comprises a nucleotide sequence that is complementary to the cryptic exon.

59. The method of claim 56, wherein the plurality of splice-switching ASOs differ with respect to formulation, chemical modification, delivery method, concentration, or amount.

60.-62. (canceled)

63. A kit comprising
a. a transgenic mouse comprising a plurality of cells which comprise a genetic sequence that is transcribed as a pre-mRNA which comprises a first segment encoding a first portion of a reporter protein, a cryptic sequence, and a second segment encoding a second portion of the reporter protein; or
b. a plurality of cells which comprise a genetic sequence that is transcribed as a pre-mRNA which comprises a first segment encoding a first portion of a reporter protein, a cryptic sequence, and a second segment encoding a second portion of the reporter protein;
and instructions for assessing a feature of an ASO by detecting the reporter protein according to the method of claim 1.

64. (canceled)


SEQUENCE LISTING (22 sequences)

SEQ ID NO: 1 - DNA, 850 bp, synthetic construct
  source 1..850: mol_type = other DNA
gtgagtctatgggacgcttgatgttttctttccccttcttttctatggttaagttcatgtcataggaaggggataagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaagtgcaatatctctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacag

SEQ ID NO: 2 - DNA, 23 bp, synthetic construct
  source 1..23: mol_type = other DNA
actccagtctttctagaagatgg

SEQ ID NO: 3 - DNA, 19 bp, synthetic construct
  source 1..19: mol_type = other DNA
cttccctcgtgatctgcaa

SEQ ID NO: 4 - DNA, 24 bp, synthetic construct
  source 1..24: mol_type = other DNA
tttctaggtaaccgatatccctgc

SEQ ID NO: 5 - DNA, 21 bp, synthetic construct
  source 1..21: mol_type = other DNA
gctatgaactaatgaccccgt

SEQ ID NO: 6 - DNA, 20 bp, synthetic construct
  source 1..20: mol_type = other DNA
ggggctcgatcctctagttg

SEQ ID NO: 7 - DNA, 19 bp, synthetic construct
  source 1..19: mol_type = other DNA
cgcgccggctagaagatgg

SEQ ID NO: 8 - DNA, 20 bp, synthetic construct
  source 1..20: mol_type = other DNA
acctgttcaattcccctgca

SEQ ID NO: 9 - DNA, 20 bp, synthetic construct
  source 1..20: mol_type = other DNA
gccttgggtcatgtcctacc

SEQ ID NO: 10 - DNA, 20 bp, synthetic construct
  source 1..20: mol_type = other DNA
tagggaggtcgcagtatctg

SEQ ID NO: 11 - DNA, 20 bp, synthetic construct
  source 1..20: mol_type = other DNA
aacaagtgctccatgctgga

SEQ ID NO: 12 - DNA, 22 bp, synthetic construct
  source 1..22: mol_type = other DNA
ctttagaatggtgcaaagaggc

SEQ ID NO: 13 - DNA, 22 bp, synthetic construct
  source 1..22: mol_type = other DNA
acccgctaagaacctcaagatg

SEQ ID NO: 14 - DNA, 22 bp, synthetic construct
  source 1..22: mol_type = other DNA
tgggaattgaactcaggacttc

SEQ ID NO: 15 - DNA, 22 bp, synthetic construct
  source 1..22: mol_type = other DNA
gcctcatctacaacgtcaagat

SEQ ID NO: 16 - DNA, 20 bp, synthetic construct
  source 1..20: mol_type = other DNA
gtgctcagcaagtcctaggg

SEQ ID NO: 17 - DNA, 16 bp, synthetic construct
  source 1..16: mol_type = other DNA
  modified_base 9: mod_base = m5c
  modified_base 15: mod_base = m5c
  modified_base 1..16: mod_base = OTHER; note = phosphorothioate linkages
gcaaattcctattccc

SEQ ID NO: 18 - DNA, 16 bp, synthetic construct
  source 1..16: mol_type = other DNA
  modified_base 2: mod_base = m5c
  modified_base 12..13: mod_base = m5c
  modified_base 1..16: mod_base = OTHER; note = phosphorothioate linkages
acttaccttaacccag

SEQ ID NO: 19 - DNA, 18 bp, synthetic construct
  source 1..18: mol_type = other DNA
  modified_base 10: mod_base = m5c
  modified_base 16..17: mod_base = m5c
  modified_base 1..18: mod_base = OTHER; note = phosphorothioate linkages
gagatattgcacttacct

SEQ ID NO: 20 - DNA, 18 bp, synthetic construct
  source 1..18: mol_type = other DNA
  modified_base 10: mod_base = m5c
  modified_base 16..17: mod_base = m5c
  modified_base 1..18: mod_base = OTHER; note = phosphorothioate linkages
gagatattgcacttacct

SEQ ID NO: 21 - DNA, 1570 bp, synthetic construct
  source 1..1570: mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaaggtgagtctatgggacgcttgatgttttctttccccttcttttctatggttaagttcatgtcataggaaggggataagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaagtgcaatatctctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 22 - DNA, 2503 bp, synthetic construct
  source 1..2503: mol_type = other DNA
atggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtgagtctatgggacgcttgatgttttctttccccttcttttctatggttaagttcatgtcataggaaggggataagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaagtgcaatatctctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgtaa